(19) United States
(12) Patent Application Publication
(10) Pub. No.: US 2026/0315581 A1
(43) Pub. Date: Oct. 15, 2026

(54) COMPOSITIONS AND METHODS FOR PREVENTING AND TREATING CANDIDA AURIS INFECTION

(71) Applicant: THE REGENTS OF THE UNIVERSITY OF MICHIGAN, Ann Arbor, MI (US)

(72) Inventors: Teresa O'MEARA, Ann Arbor, MI (US); Darian SANTANA, Ann Arbor, MI (US)

(21) Appl. No.: 19/485,159

(22) PCT Filed: May 17, 2024
(86) PCT No.: PCT/US2024/029846

§ 371 Date: Nov. 17, 2025

Related U.S. Application Data
(60) Provisional application No. 63/502,704, filed on May 17, 2023.
(60) Provisional application No. 63/514,470, filed on Jul. 19, 2023.

(51) Int. Cl. A61K 39/00; A61K 9/00; A61K 9/1271; A61K 9/51; A61K 38/00; A61K 39/385; A61K 45/06; A61P 31/14; C07K 14/40; C12N 15/85; C12N 15/88

(52) U.S. Cl. CPC A61K 39/0002; A61K 9/0014; A61K 9/1271; A61K 9/5123; A61K 39/385; A61K 45/06; A61P 31/14; C07K 14/40; C12N 15/85; C12N 15/88; A61K 38/00; A61K 2039/53; A61K 2039/55555; A61K 2039/6012; C12N 2310/3341; C12N 2800/22; C12N 2830/50

(57) ABSTRACT

Provided herein are compositions, polynucleotides (e.g., mRNAs), vaccines, and methods for the treatment and prevention of skin infection and/or systemic infection by Candida auris. In certain embodiments, the treatment and prevention include generating an immune response in a subject against at least a portion of Surface Colonization Factor (SCF1) from Candida auris (e.g., SEQ ID NO: 16). In some embodiments, treatment and prevention employs agents that inhibit SCF1 mRNA and/or the biotic and abiotic surface attachment function of SCF1.

### CROSS-REFERENCE TO RELATED APPLICATIONS

The present application claims priority to U.S. provisional applications 63/502,704, filed May 17, 2023, and 63/514,470, filed Jul. 19, 2023, both of which are herein incorporated by reference in their entirety.

### STATEMENT REGARDING FEDERAL FUNDING

This invention was made with government support under grant number AI169186 awarded by the National Institutes of Health. The government has certain rights in the invention.

### SEQUENCE LISTING

The text of the computer readable sequence listing filed herewith, titled “UM_41974_601_SequenceListing.xml,” created May 17, 2024, having a file size of 194,137 bytes, is hereby incorporated by reference in its entirety.

### FIELD OF THE INVENTION

Provided herein are compositions, polynucleotides (e.g., mRNAs), vaccines, and methods for the treatment and prevention of skin infection and/or systemic infection by Candida auris. In certain embodiments, the treatment and prevention include generating an immune response in a subject against at least a portion of Surface Colonization Factor (SCF1) from Candida auris (e.g., SEQ ID NO:16). In some embodiments, treatment and prevention employs agents that inhibit SCF1 mRNA and/or the biotic and abiotic surface attachment function of SCF1.

### BACKGROUND

Since its initial report in 2009, the emerging fungal pathogen Candida auris has become an increasingly common source of life-threatening infection worldwide (1, 2). C. auris is frequently reported in association with nosocomial outbreaks, a characteristic rarely described with other Candida species, and its outbreak potential is recognized as a cause for urgent concern by public health authorities (3-7). C. auris outbreaks are characterized by persistent colonization of patient skin and abiotic surfaces, which can remain positive for extensive lengths of time and serve as a source of horizontal transmission (8-14). C. auris can also readily colonize indwelling medical devices, and the presence of these devices is often reported as a risk factor for the development of invasive disease, where they may serve as a portal of entry to potentiate invasive infection (15-21). Challenges in adhering to intensive colonization screening and infection prevention measures are thought to contribute to the continually increasing rate of C. auris outbreaks (20). The ability of C. auris to robustly colonize biological and abiotic substrates is therefore central to its emergence as a global health threat.

Common to every colonization event is the initial physical association and attachment between fungal cells and the colonized substrate. For fungal pathogens, this association is largely mediated by cell surface-exposed proteinaceous adhesins (22). In Candida species, genetic expansion has repeatedly resulted in the formation of adhesin families containing genes similar in sequence and domain architecture, and adhesive functions can be either redundant or specific across family members (23, 24). C. auris encodes genes similar to members of the conserved ALS and IFF/HYR adhesin families, though these genes may have expanded independently in C. auris and so lack clear one-to-one homology with adhesins from well-characterized species. Moreover, their phenotypic importance in C. auris is not well understood (24-26).

### SUMMARY OF THE INVENTION

Provided herein are compositions, polynucleotides (e.g., mRNAs), vaccines, and methods for the treatment and prevention of skin infection and/or systemic infection by Candida auris. In certain embodiments, the treatment and prevention include generating an immune response in a subject against at least a portion of Surface Colonization Factor (SCF1) from Candida auris (e.g., SEQ ID NO:16 or 19), such by using an mRNA vaccine comprising SEQ ID NOs: 17 or 18, including where modified bases replace canonical bases in such sequence (e.g., using 5-methylcytosine or N1-methylpseudouridine (m1Ψ). In some embodiments, treatment and prevention employs agents that inhibit SCF1 mRNA and/or the biotic and abiotic surface attachment function of SCF1.

In some embodiments, provided herein are compositions comprising: an engineered polynucleotide encoding at least a portion of Surface Colonization Factor (SCF1) from Candida auris (e.g., SEQ ID NO:16 or 19) wherein the polynucleotide is at least partially optimized for enhanced expression, productive co-translational protein folding, increased stability, or a combination thereof. In certain embodiments, the polynucleotide comprises RNA, and optionally wherein the RNA is partially or fully human codon optimized. In further embodiments, the polynucleotide further comprises or encodes: a 5′ untranslated region (UTR), a 5′ cap, a 3′ UTR, a 3′ tailing sequence, or any combination thereof. In additional embodiments, the 5′ UTR, 3′ UTR, or both are heterologous to the polynucleotide encoding at least a portion of SCF1. In additional embodiments, the 3′ tailing sequence comprises a polyA tail, a polyG quartet, a stem loop sequence, a triple helix forming sequence, a tRNA-like sequence, or any combination thereof. In particular embodiments, the compositions further comprises water, a buffer, or saline solution.

In certain embodiments, the polynucleotide comprises at least one chemically modified nucleotide. In some embodiments, the at least one (e.g., 1, 2, 3, . . . 10 . . . 15 . . . or 20) chemically modified nucleotide comprises a modified uracil. In other embodiments, at least 20%, or least 40%, or at least 60%, or at least 80% of the uracil in the polynucleotide encoding at least a portion of SCF1 are chemically modified. In other embodiments, the at least one (e.g., 1, 2, . . . 10, . . . 15, or at least 20% or 60%) of the chemically modified nucleotide comprises 5-methylcytosine or N1-methylpseudouridine (m1Ψ).

In particular embodiments, the polynucleotide comprises a nucleotide sequence having: i) at least 75% (e.g., 75% . . . 85% . . . 95%) sequence identity to SEQ ID NOs: 2, 3, 15, 17, or 18, or a complement or reverse complement thereof, wherein T may be replaced by U, or modified bases are employed, and/or ii) at least 20, at least 25, at least 30, or at least 50, consecutive nucleotides from SEQ ID NOs: 2, 3, 15, 17, and 18, or a complement or reverse complement thereof, wherein T may be replaced by U, and/or modified bases are employed as recited herein (e.g., 5-methylcytosine or N1-methylpseudouridine (mli)). In some embodiments, the SCF1 comprises: i) an amino acid sequence having at least 90% or 95% identity to SEQ ID NO:1 or 4 or 16 or 19, and/or ii) at least 12, or at least 15, or at least 20, consecutive amino acids from SEQ ID NO:1 or 4 or 16 or 19. In other embodiments, the SCF1 comprises: i) an amino acid sequence having at least 90% or 95% identity to any one of SEQ ID NOs: 5-14 or 16, and/or ii) at least 12, 15, 20, 25, 30, 35, or 50 consecutive amino acids from any one of SEQ ID NOs: 5-14 or 16.

In some embodiments, provided herein is a messenger ribonucleic acid (mRNA) comprising: i) a 5′ untranslated region (UTR); ii) an open reading frame encoding at least a portion (e.g., immunogenic portion) of Surface Colonization Factor (SCF1) from Candida auris; and iii) a 3′ UTR, wherein the mRNA is at least partially optimized for enhanced expression, productive co-translational protein folding, increased stability, or a combination thereof. In particular embodiments, the mRNA is partially or fully human codon optimized. In certain embodiments, the 5′ UTR, 3′ UTR, or both are heterologous to the open reading frame encoding at least a portion of SCF1. In additional embodiments, the mRNA further comprises or encodes: a 5′ cap, a 3′ tailing sequence, or both. In other embodiments, the 3′ tailing sequence comprises a polyA tail, a polyG quartet, a stem loop sequence, a triple helix forming sequence, a tRNA-like sequence, or any combination thereof.

In certain embodiments, the mRNA comprises at least one chemically modified nucleotide. In other embodiments, the at least one chemically modified nucleotide comprises a modified uracil (e.g., 1, 2, . . . 10 . . . 15 . . . 20). In some embodiments, at least 20%, 40%, 60%, or 80% of the uracil in the open reading frame encoding at least a portion of SCF1 are chemically modified. In certain embodiments, the at least one chemically modified nucleotide comprises 5-methylcytosine or N1-methylpseudouridine (m1Ψ).

In particular embodiments, the mRNA comprises a nucleotide sequence having at least 70% . . . 80% . . . or 90% sequence identity to SEQ ID NO:3, 15, 17, or 18, and/or comprises at least 15 . . . 25 . . . 50 . . . or 75 consecutive nucleotides from SEQ ID NO:3, 15, 17, or 18. In some embodiments, SCF1 comprises: i) an amino acid sequence having at least 90% or 95% or 100% identity to SEQ ID NOs:1 or 4-14 or 16, and/or ii) at least 12 or at least 15, at least 25, or at least 50, consecutive amino acids from SEQ ID NOs:1-14 or 16 or 19.

In certain embodiments, provided herein are vaccines comprising: the compositions or mRNAs described above and herein; and at least one adjuvant, a delivery vehicle, or a combination thereof. In some embodiments, the delivery vehicle comprises a lipid nanoparticle encapsulating the composition or the mRNA. In other embodiments, the lipid nanoparticle comprises a cationic lipid, a neutral and/or non-cationic lipid, a sterol, or any combination thereof. In further embodiments, the non-cationic lipid comprises a phospholipid. In additional embodiments, the sterol comprises cholesterol or a modification or ester thereof. In other embodiments, the lipid nanoparticle comprises a polyethylene glycol (PEG)-lipid conjugate.

In some embodiments, provided herein are compositions comprising: an immunogenic protein comprising at least a portion of Surface Colonization Factor (SCF1) from Candida auris, wherein the immunogenic protein is conjugated to a hapten, and/or wherein the composition further comprises an adjuvant and/or a delivery vehicle. In additional embodiments, the immunogenic protein comprises: i) an amino acid sequence having at least 90% or 95% or 100% identity to SEQ ID NO:1 or 4 or 16, and/or ii) at least 10 or 12 or at least 15 or at least 20, or at least 30 consecutive amino acids from SEQ ID NO:1 or 4 or 16 or 19. In other embodiments, the SCF1 comprises: i) an amino acid sequence having at least 90% or 95% or 100% identity to any one of SEQ ID NOs: 5-14 or 16, and/or ii) at least 12 or at least 15 or at least 20 or at least 30 consecutive amino acids from any one of SEQ ID NOs: 5-14 or 16 or 19. In certain embodiments, the hapten is selected from the group consisting of: fluorescein, biotin, digoxigenin, and dinitrophenol.

In some embodiments, provided herein are methods of treating or preventing skin infection and/or systemic infection by Candida auris comprising: administering the composition, mRNA, or vaccine to a subject in need thereof. In particular embodiments, the subject has the skin infection. In other embodiments, the subject has the systemic infection. In other embodiments, the administering comprises an initial immunization and at least one subsequent immunization.

In particular embodiments, provided herein are methods of inducing an immune response in a subject comprising: administrating the composition, mRNA, or vaccine as described above and herein, to a subject in need thereof. In certain embodiments, the subject is human (e.g., with skin or systemic infection with Candida auris). In particular embodiments, provided herein are the use of the compositions, mRNAs, or vaccines described above and herein, in the manufacture of a medicament for the treatment or prevention skin infection and/or systemic infection by Candida auris.

In certain embodiments, provided herein are methods comprising: treating a subject with a composition comprising a first agent, wherein the subject has a skin infection and/or systemic infection by Candida auris, and wherein the first agent is selected from the following: a) an anti-SCF1 antibody or antigen binding fragment thereof, b) an antisense sequence specific for SCF1 DNA or mRNA, c) a siRNA sequence specific for SCF1 mRNA; d) an antisense sequence specific for C. auris Swi1, BCY1, EFG1, IRF1, and TPK2, DNA or mRNA, e) a siRNA sequence specific for C. auris Swi1, BCY1, EFG1, IRF1, and TPK2 mRNA; and f) a small molecule that inhibits the function of one of the following proteins of C. auris: SCF1, BCY1, EFG1, IRF1, and TPK2.

In some embodiments, the subject has the skin infection. In other embodiments, the treating comprises applying the composition topically to the skin infection of the subject (e.g., as a cream or a spray). In further embodiments, the treating comprises providing the composition to the subject such that the subject is able to apply the composition to the skin infection (e.g. as a cream or a spray). In particular embodiments, the applying the composition, or providing the composition, is repeated daily for at least 7 days, or for at least 14 days, or for at least 21 days. In other embodiments, the composition further comprises a hydrogel agent, and the composition is in the form of a hydrogel. In other embodiments, the the composition further comprises a solubilizer and/or emulsifier. In additional embodiments, the composition is in the form of a cream, emulsion, skin lotion, gel, an ointment, a spray, or is present in a skin patch. In particular embodiments, the subject is a human. In other embodiments, the subject has a systemic infection with C. auris. In some embodiments, the treating comprises intravenous injection of the composition.

In other embodiments, provided herein are methods comprising: treating a surface of an object with a composition comprising a first agent, wherein the surface of the object is suspected of harboring Candida auris, and wherein the first agent is selected from the following: a) an anti-SCF1 antibody or antigen binding fragment thereof, b) an antisense sequence specific for SCF1 DNA or mRNA, c) a siRNA sequence specific for SCF1 mRNA; d) an antisense sequence specific for C. auris Swi1, BCY1, EFG1, IRF1, and TPK2, DNA or mRNA, e) a siRNA sequence specific for C. auris Swi1, BCY1, EFG1, IRF1, and TPK2 mRNA; and f) a small molecule that inhibits the function of one of the following proteins of C. auris: SCF1, BCY1, EFG1, IRF1, and TPK2. In particular embodiments, the composition is in the form of a foam or liquid, and wherein the treating comprises spraying the composition on the surface of the object. In other embodiments, the composition further comprises a solubilizer and/or emulsifier. In some embodiments, the object comprises medical equipment or a surgical bed. In other embodiments, the medical equipment is selected from: a surgical tool, catheter, a kidney tray, surgical scissors, and surgical clamps. In certain embodiments, the small molecule is selected from: balanol, an acyclic balanol analog, PKA-IN-1, H-8 dihydrochloride, Gliotoxin, AT7867, CCT128930, A-674563, AT13148, GSK690693, BRD-K98645985, and AU-15330.

### DESCRIPTION OF THE FIGURES

FIG. 1. Surface Colonization Factor (SCF1) mediates C. auris adhesion to polymer surfaces. (A) Adhesion of wild type AR0382 or mutants lacking one of twelve genes from ALS or IFF/HYR adhesin families. (B) 2,560 insertional mutants in the AR0382 strain background were screened for adhesion defects by measuring the proportion of cells able to remain attached to a cyclic olefin polymer surface after 3 washes with PBS. Strains are ordered by Z-score rank. Mutants with a Z-score more negative than −3 were considered to have a significant adhesive defect. (C) Adhesion of AR0382 and an insertional SWI1 mutant. (D) RNA-seq comparing the transcriptome of tnSWI1 to AR0382. SCF1 (B9J08_001458) is the strongest dysregulated gene. (E) Predicted domain architecture of Scf1, based on the clade I primary sequence, is consistent with canonical fungal adhesins. (F) Adhesion of adhesin mutants and complements compared to AR0382 (G) Immunofluorescence microscopy using an c-FLAG antibody. Representative images shown for WT AR0382 and AR0382 Ascf1+SCF1-FLAG. Scale bar=5 μm. Statistical differences were assessed using one-way ANOVA with Dunnett's post-hoc test (A), student's t-test (C), or one-way ANOVA with Tukey's post-hoc test (G); *p≤0.05; **p≤0.01; ***p≤0.001; ns: p>0.05.

FIG. 2. C. auris uniquely relies on SCF1 for adhesive plasticity. (A) Synteny schema depicting SCF1 and the conservation and orientation of adjacent ORFs. Genomic loci are shown in comparison to C. auris. Putative SCF1 homologs were only identified in C. auris and C. haemulonii. (B) Adhesion of 23 C. auris clinical isolates from all 5 clades. (C) Adhesion of 19 C. albicans clinical isolates from five clades. FC=Fingerprint Clade. (D) SCF1 transcript abundance (top panel) but not IFF4109 transcript abundance (bottom panel) is associated with adhesion to polystyrene in the same 23 C. auris isolates from (A). Log 2FC are expressed relative to AR0382. Each point signifies the mean of three biological replicates. Pearson correlation coefficient and p-value indicated. Isolates that do not encode IFF4109 are not indicated in the bottom panel. (E) Comparison of adhesion between two Clade I isolates: AR0382 and AR0387. Overexpression of SCF1 using the strong TEF1 promoter (right panel) is sufficient to drive adhesion in the poorly adhesive AR0387 background (left panel). Statistical differences were assessed using one-way ANOVA (A) and (B), with Tukey's post-hoc test (D) or student's t-test (D); *p≤0.05; **p≤0.01; ***p≤0.001; ns: p>0.05.

FIG. 3. IFF4109, but not SCF1, mediates adhesion through cell surface hydrophobicity. (A) Representative images from Microbial Attachment to Hydrocarbons (MATH) assay. Hydrophobic cells are sequestered from the aqueous phase (Aq) to the aqueous-hydrocarbon interface (Int) after mixing with the hydrocarbon phase (HC). (B) Proportion of cells sequestered out of the aqueous phase during MATH assay. (C), (D) Cells were allowed to attach to a hydrophobic, untreated polystyrene surface (C) or a hydrophilic, vacuum plasma treated polystyrene surface (D) for 1 hour. The surface was washed and the proportion of cells that remained attached after washing was measured. Statistical differences were assessed using one-way ANOVA with Tukey's post-hoc test (B), (C), and (D) or student's t-test (C) and (D); *p≤0.05; **p≤0.01; ***p≤0.001; ns: p>0.05.

FIG. 4. Specific cationic residues are critical for Scf1-mediated surface association (A), (B) WT AR0382 adhesion in the presence of increasing concentrations of arginine (A) or 1 M additives (B). (C) Predictive model of the Scf1 N-terminal domain with two neighboring cationic-aromatic clusters highlighted. (D) Adhesion of wild type AR0382, a mutant lacking SCF1, or AR0382 Ascf1+SCF1-FLAG mutants encoding the wild type SCF1 allele or alleles containing the indicated mutations. (E) TMR-labelled 13-amino acid peptides corresponding to the wild type Scf1 sequence (residues 50-62) or the same sequence with the indicated mutations incubated with the same polystyrene microspheres used to measure adhesion in (D). Scale Bar=5 μm. (F) Quantification (MFI) of peptide binding to individual polystyrene microspheres as in (E) measured by TMR epifluorescence, corrected for background fluorescence. Each point represents an individual microsphere. Colored points represent averages of individual experiments, used for statistical analysis. Statistical differences were assessed using one-way ANOVA with Dunnett's post-hoc test (A), (B) or one-way ANOVA with Tukey's post-hoc test (D), (F); *p≤0.05; **p≤0.01; ***p≤0.001; ns: p>0.05.

FIG. 5. SCF1 mediates host colonization and infection phenotypes. (A), (B) Polyethylene central venous catheters were set in rat jugular veins and inoculated intraluminally with C. auris. Representative scanning electron microscopy images of the luminal catheter surface are shown from catheters collected 24 hrs after infection. Scale bars: 400 μm (100×), 40 μm (1000×), 5 μm (5000×). (C), (D) Full thickness human skin explants were colonized with C. auris for 24 hrs before washing to remove unassociated cells. Representative scanning electron microscopy images of the skin surface following washing are shown. Scale bars: 20 μm (1000×), 4 μm (5000×). (E), (F), (G) Immunosuppressed mice were infected intravenously (via tail vein injection) with 5×10⁷ C. auris cells. Histopathology sections of the kidneys 7 days post infection (E), (F) were stained with PAS. Magenta color indicates lesion areas. Ten infected mice for each strain were monitored for survival for 21 days (G). Statistical comparisons of overall survival were assessed using the Mantel-Haenszel log-rank test with Benjamini-Hochberg correction. *p≤0.05; **p≤0.01; ***p≤0.001; ns p>0.05.

FIG. 6. Gating strategy for dispersed surface flow cytometry adhesion assay. Cells were gated from fluorescent microspheres using forward scatter. Ten-thousand gated events were collected for cells and divided into two populations using FITC fluorescence. Attached cells were identified by positive fluorescent signal. Attachment was expressed as the proportion of attached cells to the total population.

FIG. 7. A BCY1 insertional mutant shows reduced adhesion associated with downregulation of SCF1 but not IFF4109. Adhesion to polystyrene microspheres and transcript abundance compared to the parental AR0382 for SCF1 and IFF4109. Statistical differences were assessed using student's t-test; *p≤0.05; **p≤0.01; ***p≤0.001; ns: p>0.05.

FIG. 8. Individual adhesin genes exhibit distinct phenotypes. (A) Adhesion to polystyrene microspheres measured for C. albicans SC5314 or a mutant lacking ALS1. Cells were grown in yeast phase in YPD at 30° C. (B) DESeq-normalized read counts for all ORFs based on AR0382 RNA-seq. Read counts for adhesins are indicated. Horizontal lines mark the 5^{th}, 25^{th}, 50^{th}, 75^{th}, and 95^{th} percentiles among all ORFs. (C) Flocculation activity measured over time for wild type AR0382 or mutants lacking SCF1 or driving ALS 4112 with the C. auris TEF1 promoter. Data are mean±SEM from three replicates. (D) Representative brightfield microscopy (top) and photographs of broth culture after 20 minutes of settling (bottom) for the strains assessed in (C). Scale bar=10 μm. Statistical differences were assessed using student's t-test (A); *p≤0.05; **p≤0.01; ***p≤0.001; ns: p>0.05.

FIG. 9. C. haemulonii does not rely on SCF1 for adhesion. (A) Adhesion of C. auris AR0382 Δscf1 mutant or the same mutant complemented with the SCF1 allele from C. auris AR0382 or from C. haemulonii AR0395. (B) Adhesion of C. haemulonii AR0395 and a mutant lacking the putative SCF1 homolog (CXQ85_003100). (C) Adhesion of three distinct C. haemulonii isolates and SCF1 expression compared to AR0393. Statistical differences were assessed using student's t-test (B) or one-way ANOVA with Tukey's post-hoc test (A) and (C); *p≤0.05; **p≤0.01; ***p≤0.001; ns: p>0.05.

FIG. 10. SCF1 is the most strongly dysregulated gene between AR0387 and AR0382. (A) Transcriptome of AR0387 compared to AR0382. Genes in red are significantly dysregulated in AR0387, with SCF1 being the most strongly and most significantly downregulated gene. (B) Comparison of the downregulated transcriptome between two poorly adhesive strains compared to the strongly adhesive AR0382. The regulon of AR0387 and tnSW11 overlap by 14 genes.

FIG. 11. Different SCF1 alleles exhibit different magnitudes of attachment in a strain-dependent manner. (A) Overexpression of the AR0382 (Clade I) SCF1 allele in AR0381 (Clade II) confers a stronger adhesive phenotype than overexpression of the endogenous AR0381 SCF1 allele (left panel), despite similar transcript abundance (right panel). (B) The AR0382 Δscf1 attachment defect is fully rescued by complementation with the SCF1 allele from either AR0382 or AR0381. Statistical differences were assessed using one-way ANOVA with Tukey's post-hoc test; *p≤0.05; **p≤0.01; ***p≤0.001; ns: p>0.05.

FIG. 12. Loss of either SCF1 or IFF4109 does not result is transcriptional dysregulation of the other. qPCR comparing transcript abundance for IFF4109 or SCF1 in strains lacking SCF1 or IFF4109 compared to the parental wild type. Statistical differences were assessed using student's t-test; *p≤0.05; **p≤0.01; ***p≤0.001; ns: p>0.05.

FIG. 13. The SCF1 N-terminal domain contains a FLO11-like Fibronectin-type III fold but lacks conserved aromatic residues important to Flo11 function. (A)N-terminal domain models for S. cerevisiae Flo11 (crystal structure) and C. auris Scf1 (AlphaFold2 predictive model) and membrane topology diagrams. Aromatic bands that are conserved and have functional roles in Flo11 homologues throughout Ascomycota are highlighted. The central Fibronectin-type III fold is highlighted in the topology diagrams. (B) Primary sequence alignment between S. cerevisiae Flo11 N-terminal domain and C. auris Scf1 N-terminal domain. Identical residues are boxed in red; similar residues are boxed in white. Inverted triangles indicate the positions of aromatic residues that are conserved from S. cerevisiae to Flo11 homologues in other Ascomycotal species. Red triangles indicate residues that have demonstrated functional roles in biofilm formation, invasive growth, or homotypic interactions.

FIG. 14. Predictive model confidence for the Scf1 N-terminal domain is substantially reduced outside of the Fibronectin-type III fold. N-terminal domain models for C. auris Scf1 (AlphaFold2 predictive model) and the domain sequence colored by per-residue pLDDT score. Cutoffs are set according to AlphaFold2 conventions. Numbers indicate amino acid position from the start codon.

FIG. 15. Specific cationic residues are critical to the ability of Scf1 to mediate surface association. (A) Predictive model of the Scf1 N-terminal domain with hypothesized critical cationic residues highlighted in red and neighboring aromatic residues highlighted in blue. (B) Adhesion to polystyrene microspheres measured for wild type AR0382, a mutant lacking SCF1, or AR0382 Ascf1+SCF1-FLAG mutants encoding the wild type SCF1 allele or alleles containing the indicated mutations. (C) Relative SCF1 transcript abundance by RT-qPCR compared to wild type AR0382 levels. No significant differences were identified. (D) Representative fluorescence intensity of strains labeled with cc-FLAG antibody. Each plot represents 50,000 events captured by flow cytometry. (E) MFI quantification of replicates from labelled cells as in (B). (F) Brightfield and epifluorescent microscopy of cells labeled with α-FLAG antibody. Scale bar=5 μm. Statistical differences were assessed using one-way ANOVA with Dunnett's post-hoc test using AR0382 as comparator (B), (C) or AR0382 Δscf1+SCF1-FLAG_{WT} as comparator (E); *p≤0.05; **p≤0.01; ***p≤0.001; ns: p>0.05.

FIG. 16: Scf1 mediates lipid binding. (A) TMR-labelled 13-amino acid peptides corresponding to Sefl residues 50-62 or the same sequence with indicated mutations were incubated with phosphotidyl choline (PC) microparticles with exposed phospholipid head groups. Scale bar=10 μm. (B) MFI of PC lipid particles bound by peptides as in (A), normalized to background fluorescence. Each point represents an individual particle. Colored points represent averages from individual experiments, used for statistical analysis. (C) Fraction of PC lipid particles bound by cells after incubation with cultures of each of the indicated strains. Statistical differences were assessed using one-way ANOVA with Tukey's post-hoc test; *p≤0.05; **p≤0.01; ***p≤0.001; ns: p>0.05.

FIG. 17: SCF1 drives biofilm formation in vitro. Biofilms were grown on polystyrene in RPMI at 37° C. and stained with calcofluor white. Representative biofilm images in (A), (B), and (D) are depicted as maximum intensity projections from Z-stacks captured using epifluorescence microscopy. (A) Deletion of both SCF1 and IFF4109 is required to abrogate biofilm formation in AR0382. (B) Overexpression of SCF1 is sufficient to drive biofilm formation in AR0387. (C) Oxidative activity of in vitro biofilms was measured using an XTT-reduction assay. (D) Expression of SCF1 in a clade II isolate is sufficient to drive biofilm formation. Representative images of biofilms formed by wild type AR0381 (clade II) or mutants overexpressing either the endogenous AR0381 SCF1 allele or the clade I SCF1 allele from AR0382. (E) Oxidative activity of clade II biofilms was measured using an XTT-reduction assay. Scale bar=20 μm. Statistical differences were assessed using one-way ANOVA with Tukey's post-hoc test; *p≤0.05; **p≤0.01; ***p≤0.001; ns: p>0.05.

FIG. 18: SCF1 is critical for skin colonization. (A) C. auris cells were incubated on full thickness ex vivo human skin samples for 24 hrs before washing and measuring remaining bioburden by colony forming units (CFU). (B) Viable burden of C. auris after 2 days incubation on murine skin in vivo. Data are presented as CFU/g tissue normalized to actual delivered inocula. Statistical differences were assessed using one-way ANOVA with Tukey's post-hoc test; *p≤0.05; **p≤0.01; ***p≤0.001; ns: p>0.05.

FIG. 19: SCF1 is critical for dissemination and fungal burden. Histopathology sections of the heart of C. auris-infected mice were stained with PAS and imaged at 2× and 20× resolution using Olympus bright field microscopy. Lower panel for each organ represents the magnified area of colored boxes from 2× panel. Magenta color indicates lesion areas.

FIG. 20A shows the Scf1 full length amino acid sequence (SEQ ID NO: 1). FIG. 20B show the Scf1 full length DNA sequence (SEQ ID NO:2).

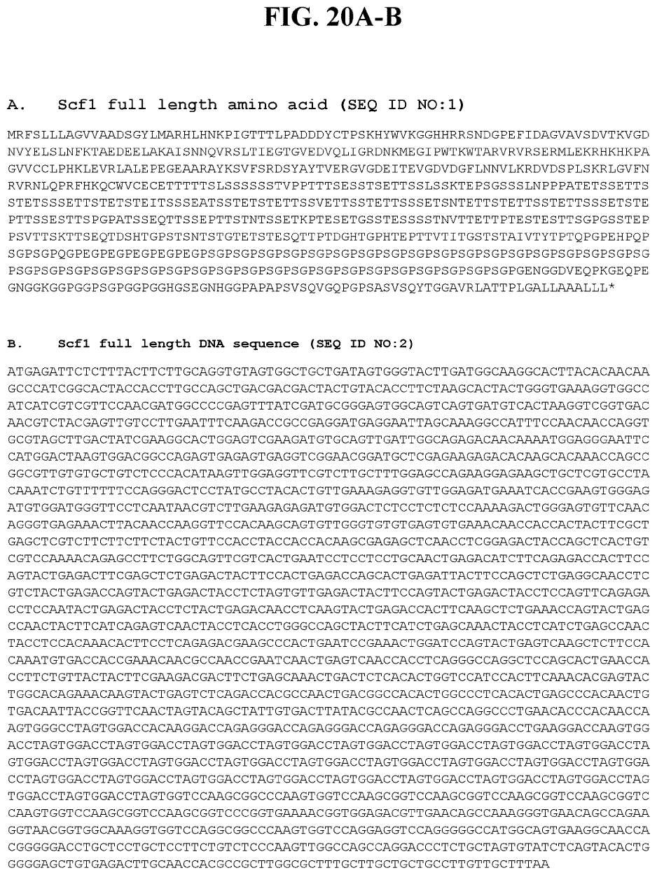

FIG. 21A shows the Scf1 full length coding mRNA sequence (SEQ ID NO:3). FIG. 21B shows an exemplary human codon optimized Scf1 full length mRNA sequence (SEQ ID NO:15).

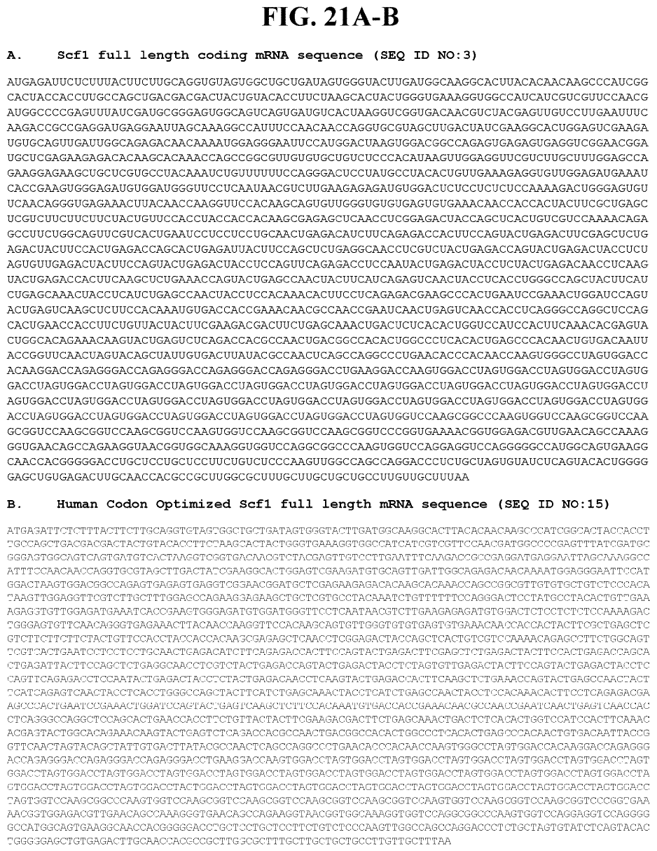

FIG. 22A shows the Scf1 N-terminal domain amino acid sequence (res 28-254) (SEQ ID NO:4). FIG. 22B shows the Scf1 N-terminal truncated peptide amino acid sequence (res 28-60) (SEQ ID NO:5). FIG. 22C shows the Scf1 nonconserved loop amino acid sequence (res 120-162) (SEQ ID NO:6). FIG. 22D shows the Scf1 central fold amino acid sequence (res 61-145) (SEQ ID NO:7). FIG. 22E shows the Scf1 helicies amino acid sequence (res 163-217) (SEQ ID NO:8). FIG. 22F shows a Scf1 truncated peptide (res 50-62) (SEQ ID NO:16). FIG. 22G shows the mRNA sequence encoding truncated peptide 50-62 (SEQ ID NO:17). FIG. 22H shows the mRNA sequence encoding truncated peptide 50-62 (SEQ ID NO:18).

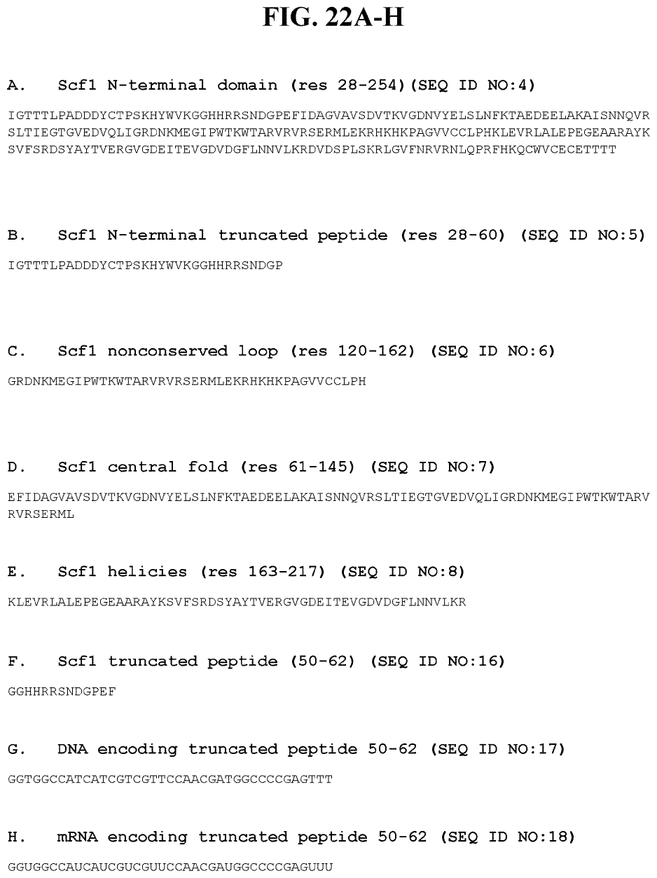

FIG. 23 shows a hypothetical model for how SCF1 is regulated. It is noted that the present disclosure is not dependent on this hypothetical model and can be practiced without an understanding of the regulation of the SCF1 gene.

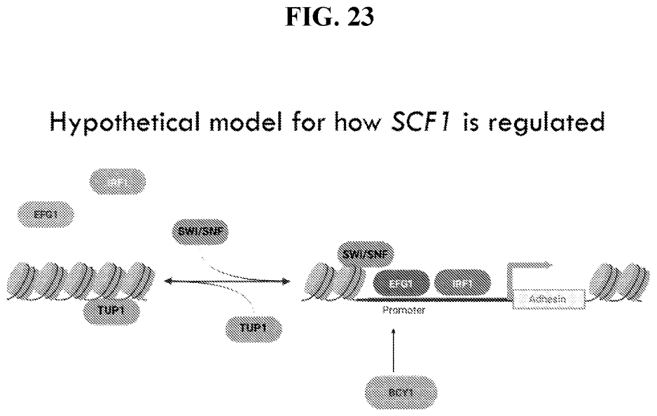

FIG. 24. It is known that Tup1 is a repressor of filamentation and adherence in C. albicans. Deleting Tup1 in the low adherent C. auris strain increased attachment through SCF1 expression (FIG. 21A) because we can see that SCF1 expression is much higher in the tup1 mutant (FIG. 21B).

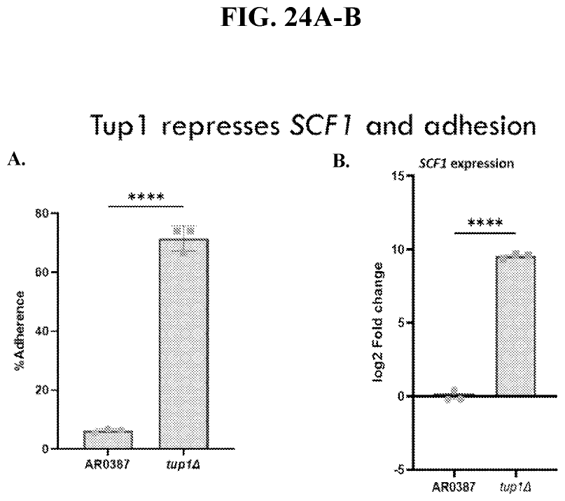

FIG. 25. A forward genetics screen identified Bcy1 as a regulator of C. auris adhesion. A) 2,560 insertional mutants in the AR0382 strain background were screened for adhesion defects by measuring the proportion of cells able to remain attached to a cyclic olefin polymer surface after 3 washes with PBS. Strains are ordered by Z-score rank. Mutants with a Z-score more negative than −3 were considered to have a significant adhesive defect. B) Insertion in Bcy1 results in a defect in adherence.

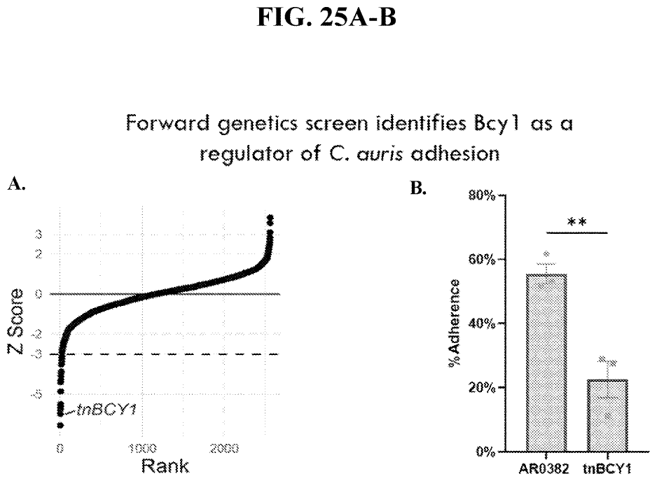

FIG. 26 shows Protein Kinase A (PKA) pathway. It is known that Bcy1 is the regulatory subunit of PKA, which controls the phosphorylation of transcription factors.

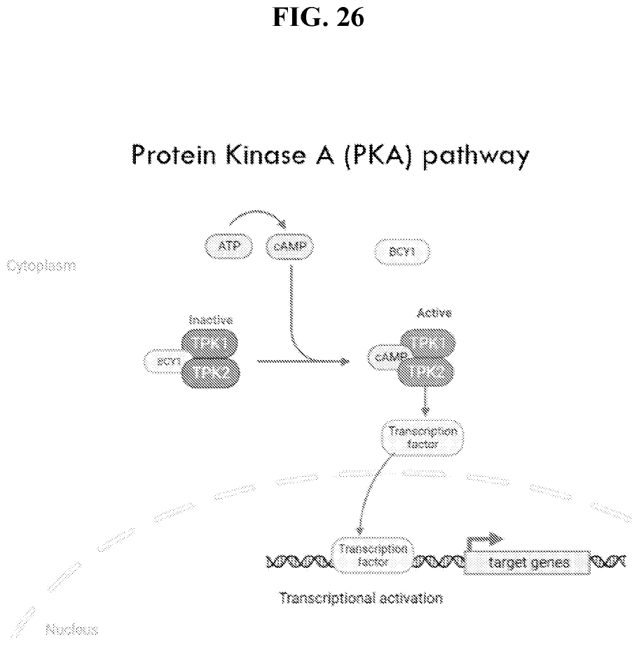

FIG. 27 shows that hyperactivation of PKA leads to adhesion defect. This was confirmed by making a Bcy1 deletion, which led to a decrease in attachment corresponding to SCF1 expression (FIG. 27A) and complementation led to a rescue of the phenotype (FIG. 27B) also corresponding to SCF1 expression

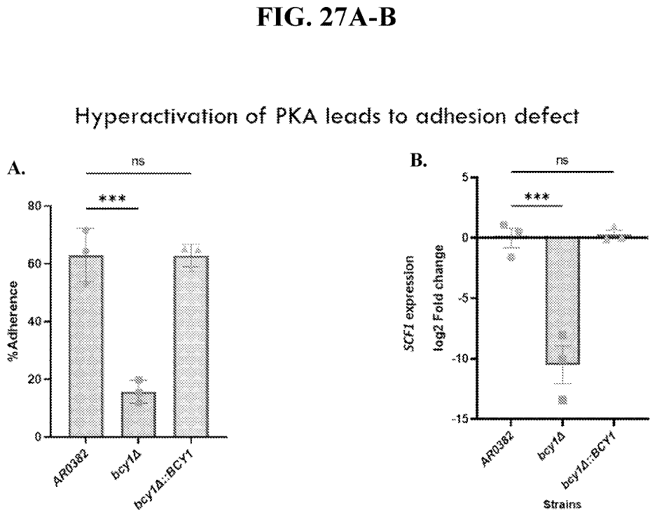

FIG. 28 shows that TPK1, surprisingly, does not regulate adhesion of C. auris. A) Mutation of tpk1 in AR0382 does not impact attachment. B) Mutation of tpk1 in AR0387does not impact attachment C) tpk1 mutants do not change SCF1 expression, as measured through qRT-PCR.

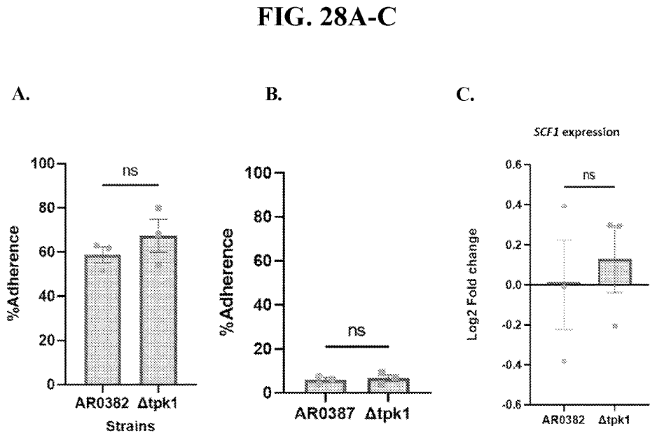

FIG. 29 shows that EFG1 regulates adherence through SCF1, which was surprising. This was determined by making a Efg1 deletion mutation, which lead to a decrease in in attachment (FIG. 29A) and decrease in SCF1 expression (FIG. 29B).

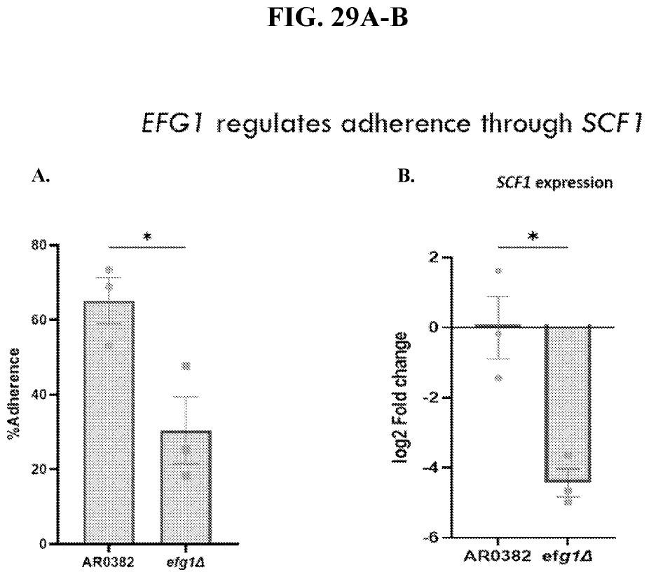

FIG. 30 shows release of Tup1 repression of SCF1 requires Efg1, however the main transcription factor regulating adhesion in the tup1 deletion strain as the double mutant is no longer able to adhere.

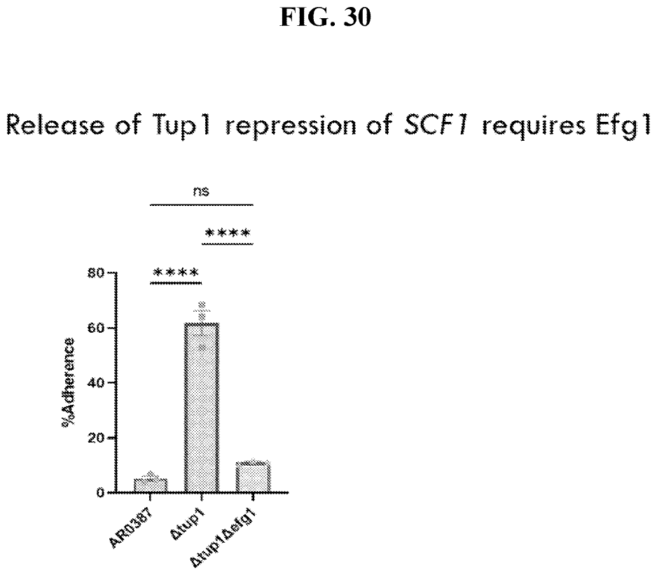

FIG. 31. A forward genetics screen also identified IRF1, a non-canonical transcription factor, that in C. abicans is involved in regulation of iron homeostasis and morphogenesis. A) Attachment assays for irf1 mutant strains compared with the parental isolates. B) SCF1 expression in irf1 mutant strains compared with the parental isolate

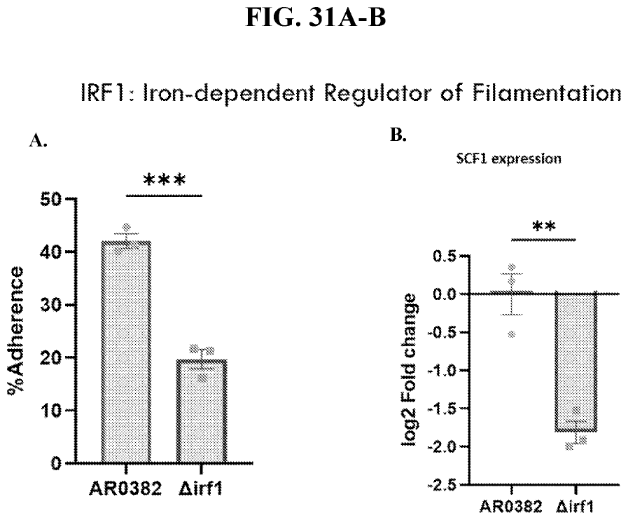

FIG. 32 shows expression of IRF1 is dependent on TUP1 & SWI1. A) qRT-PCR analysis of IRF1 expression in the Tup1 mutant strains, compared with their parental isolates. B) RNAseq shows decreased SCF1 expression in the tnSWI1 mutant strain.

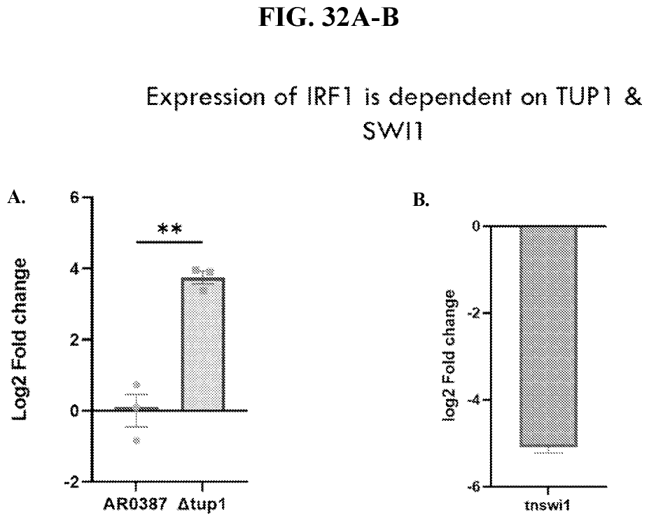

### DEFINITIONS

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include Q-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

The term “immunization,” as used herein, refers to a process that increases an organisms' reaction to antigen and therefore improves its ability to resist or overcome infection.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. The nucleic acid, whether DNA or RNA may comprise non-natural nucleotides, modified nucleotides, and/or non-nucleotide building blocks that can exhibit the same function as natural nucleotides (e.g., “nucleotide analogs”). Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. Peptides and polypeptides include proteins such as binding proteins, receptors, and antibodies. The terms “peptide,” “polypeptide” and “protein,” are used interchangeably herein.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature, and/or the nucleic acid molecule or the polypeptide is associated with at least one other component with which it is not naturally associated in nature and/or that there is one or more changes in nucleic acid or amino acid sequence as compared with such sequence as it is found in nature.

The term “vaccine,” as used herein, refers to any pharmaceutical composition containing at least one antigenic or immunogenic peptide or other immunogen or at least one nucleic acid encoding at least one antigenic or immunogenic peptide or other immunogen, which can be used to prevent or treat a disease or condition in a subject.

As used herein, the term “preventing” refers to partially or completely delaying onset of an infection; partially or completely delaying onset of one or more symptoms, features, or manifestations of a particular infection; partially or completely delaying progression from an infection; and/or decreasing the risk of developing pathology associated the infection.

As used herein, “treat,” “treating,” and the like means a slowing, stopping, or reversing of progression of a disease or disorder when provided a composition or vaccine described herein to an appropriate subject.

A “subject” or “patient” may be human or non-human and may include, for example, animal strains or species used as “model systems” for research purposes. Likewise, subject may include either adults or juveniles (e.g., children), or males or females. Moreover, subject may mean any living organism, preferably a mammal (e.g., a human or a non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used herein, the terms “administering,” “providing,” and “introducing,” are used interchangeably herein and refer to the placement of the compositions, polynucleotides (e.g., mRNA), or vaccines of the disclosure into a subject. Administration may be by any appropriate route which results in delivery to a desired location in the subject.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 degrees Celsius in a solution consisting of 5×SSPE (43.8 g/l NaCl, 6.9 g/l NaH2PO4·H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 degrees Celsius when a probe of about 500 nucleotides in length is employed.

“Medium stringency conditions” when used in reference to nucleic acid hybridization comprise conditions equivalent to binding or hybridization at 42 degrees celsiuis in a solution consisting of 5×SSPE (43.8 g/l NaCl, 6.9 g/l NaH2PO4·H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 degrees C. when a probe of about 500 nucleotides in length is employed.

“Low stringency conditions” comprise conditions equivalent to binding or hybridization at 42 degrees Celsius in a solution consisting of 5×SSPE (43.8 g/l NaCl, 6.9 g/l NaH2PO4·H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42 degrees Celsius when a probe of about 500 nucleotides in length is employed.

### DETAILED DESCRIPTION

Provided herein are compositions, polynucleotides (e.g., mRNAs), vaccines, and methods for the treatment and prevention of skin infection and/or systemic infection by Candida auris. In certain embodiments, the treatment and prevention include generating an immune response in a subject against at least a portion of Surface Colonization Factor (SCF1) from Candida auris. In some embodiments, treatment and prevention employs agents that inhibit SCF1 mRNA and/or the biotic and abiotic surface attachment function of SCF1.

In work conducted during development of embodiments herein, to interrogate the role of individual C. auris adhesins in colonization phenotypes, we measured the attachment of fungal cells to polymer substrates as a model for surface association. We demonstrate that C. auris does not substantially rely on conserved ALS or IFF/HYR family adhesins for surface attachment. Instead, we identified Surface Colonization Factor (SCF1), an adhesin specific to C. auris with homologs only present in C. auris and the closely related Candida haemulonii species. SCF1 is necessary and sufficient for robust attachment of C. auris cells to polymer substrates. C. auris isolates from diverse and similar genetic lineages exhibit striking divergence in terms of substrate association, and this phenotypic plasticity is tightly correlated with strain-specific transcriptional control of SCF1. While the present disclosure is not limited by any particular mechanism, and an understanding of the mechanism is not necessary to practice the invention, work conducted during development of embodiments herein suggests the nonspecific surface association driven by SCF1 does not occur through hydrophobic interactions like for many conserved fungal adhesins, but rather through cation-substrate interactions analogous to adhesion mechanisms exhibited by marine organism adhesive proteins. To explore the clinical relevance of these findings, we investigated the importance of SCF1 in long-term colonization models. We found SCF1 to be critical for biofilm formation in vitro, robust colonization of central venous catheters, colonization of human skin, and virulence in systemic infection, and that the differential utilization of SCF1 between otherwise related clinical isolates explained naturally varying differences in these phenotypes. These findings offer insight into the unique genetic and molecular mechanisms by which C. auris mediates surface colonization, a trait important to the disease burden of this emerging pathogen.

### I. SCF1 Peptides, Polynucleotides, and Compositions

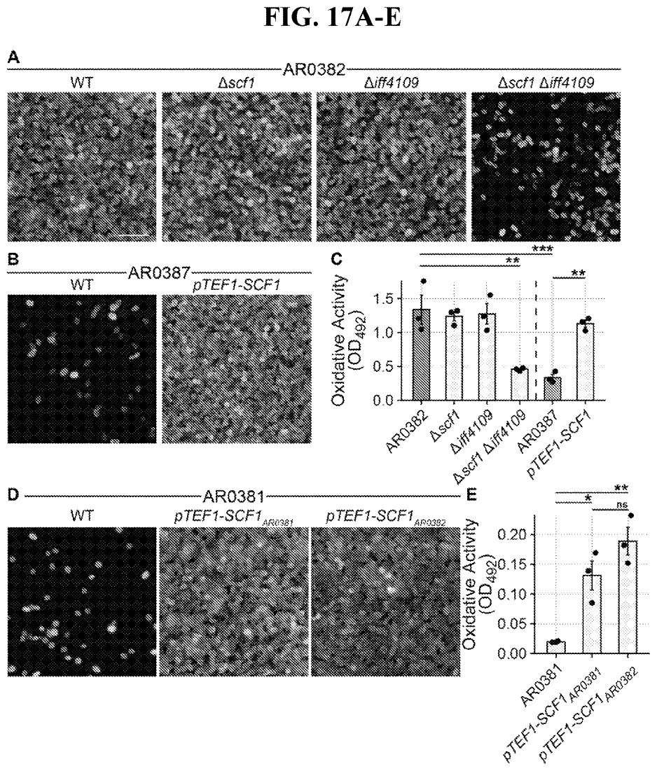

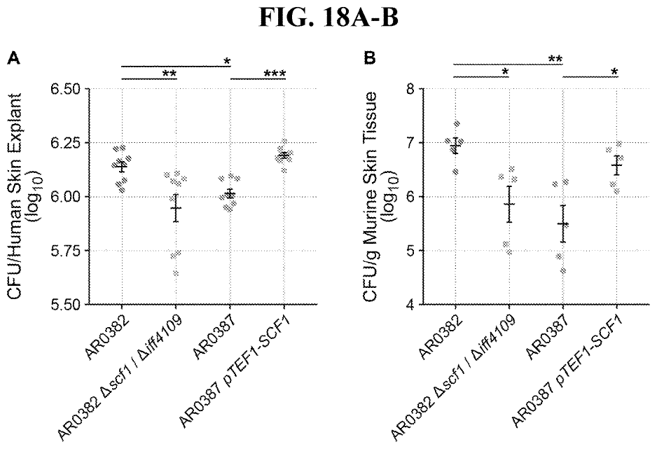

In some embodiments, provided herein are polynucleotide sequences that encode at least a portion of Surface Colonization Factor (SCF1) from Candida auris (e.g., SCF1 peptides). Such SCF1 polynucleotides and peptides are shown, for example, in FIGS. 17-19, which show SEQ ID NOs: 1-8 and 15-18, as well as SEQ ID NOs:9-14. Other embodiments provide fragments, fusion proteins or functional equivalents of these peptides. In some embodiments, the SCF1 polynucleotides and peptides may be a purified product, in other embodiments they may be a product of chemical synthetic procedures, and in still other embodiments they may be produced by recombinant techniques using a prokaryotic or eukaryotic host (e.g., by bacterial, yeast, higher plant, insect and mammalian cells in culture).

In certain embodiments, due to the inherent degeneracy of the genetic code, DNA sequences other than the polynucleotide sequences of SEQ ID NOs: 2, 3, 15, 17, or 18 which encode substantially the same or a functionally equivalent amino acid sequence (or immunogenic fragments thereof, such as from the N-terminal), may be used to clone and express SCF1 peptides. In general, such polynucleotide sequences hybridize to SEQ ID NOs: 2, 3, 15, 17, or 18 under conditions of high to medium stringency as described above. As will be understood by those of skill in the art, it may be advantageous to produce SCF1 encoding nucleotide sequences possessing non-naturally occurring codons. Therefore, in some preferred embodiments, codons preferred by a particular prokaryotic or eukaryotic host (Murray et al., Nucl. Acids Res., 17 [1989]) are selected, for example, to increase the rate of SCF1 expression or to produce recombinant RNA transcripts having desirable properties, such as a longer half-life, than transcripts produced from naturally occurring sequence.

In certain embodiments, the peptides comprises or consist of the peptides in Table 6 below, or variants thereof with 1, 2, 3, or 4 amino acid changes. Such peptides, for example, may be used as capture peptides and/or immunogens to generate antibodies.

TABLE 6

| Amino Acid Sequence | SEQ ID NO: |
|---|---|
| IGTTTLPADDDYCTPSKHYWVKGGHHRRSNDGPEFIDAGVAVSDVTKVGDN | 4 |
| VYELSLNFKTAEDEELAKAISNNQVRSLTIEGTGVEDVQLIGRDNKMEGIP | |
| WTKWTARVRVRSERMLEKRHKHKPAGVVCCLPHKLEVRLALEPEGEAARAY | |
| KSVFSRDSYAYTVERGVGDEITEVGDVDGFLNNVLKRDVDSPLSKRLGVEN | |
| RVRNLQPRFHKQCWVCECETTTT | |
| IGTTTLPADDDYCTPSKHYWVKGGHHRRSNDGP | 5 |
| LPADDDYCTPSKHYWVKGGHHRRSNDGP | 9 |
| IGTTTLPADDDYCTPSKHYWVKGGHHRR | 10 |
| LPADDDYCTPSKHYWVKGGHHRR | 11 |
| DDDYCTPSKHYWVKGGHHRR | 12 |
| LPADDDYCTPSKHYWVKGGH | 13 |
| DDDYCTPSKHYWVKGGH | 14 |
| GHHRRSNDGPE | 19 |

The polynucleotides described herein may be employed for producing SCF1 polypeptides by recombinant techniques. Thus, for example, the polynucleotide may be included in any one of a variety of expression vectors for expressing a polypeptide. In some embodiments, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments provided herein are recombinant constructs comprising one or more of the sequences as broadly described above (e.g., SEQ ID NO: 2, 3, 15, 17, or 18 or sub-portions thereof). In some embodiments, the constructs comprise a vector, such as a plasmid or viral vector, into which a sequence of the invention has been inserted, in a forward or reverse orientation. In still other embodiments, the heterologous structural sequence is assembled in appropriate phase with translation initiation and termination sequences. In certain embodiments, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE 9 (Qiagen), pBS, pD10, phagescript, psiX174, pbluescript SK, pBSKS, pNH8A, pNH16a, pNH18A, pNH46A (Stratagene); ptrc99a, pKK223 3, pKK233 3, pDR540, pRITS (Pharmacia); pET vectors (Novagen); and 2) Eukaryotic—pWLNEO, pSV2CAT, pOG44, PXT1, pSG (Stratagene) pSVK3, pBPV, pMSG, pSVL (Pharmacia). Any other plasmid or vector may be used as long as they are replicable and viable in the host.

In certain embodiments, the DNA sequence in the expression vector is operatively linked to an appropriate expression control sequence(s) (promoter) to direct mRNA synthesis. Useful promoters include, but are not limited to, the LTR or SV40 promoter, the E. coli lac or trp, the phage lambda PL and PR, T3 and T7 promoters, and the cytomegalovirus (CMV) immediate early, herpes simplex virus (HSV) thymidine kinase, and mouse metallothionein I promoters and other promoters known to control expression of gene in prokaryotic or eukaryotic cells or their viruses. In other embodiments, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In a further embodiment, provided herein are host cells containing the above-described constructs. In some embodiments, the host cell is a higher eukaryotic cell (e.g., a mammalian or insect cell). In other embodiments, the host cell is a lower eukaryotic cell (e.g., a yeast cell). In still other embodiments, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS 7 lines of monkey kidney fibroblasts, (Gluzman, Cell 23:175 [1981]), C127, 3T3, 293, 293T, HeLa and BHK cell lines.

SCF1 peptides can be expressed in mammalian cells, yeast, bacteria, or other cells under the control of appropriate promoters. Cell free translation systems can also be employed to produce such proteins using RNAs derived from the DNA constructs described herein. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989).

The present disclosure also provides methods for recovering and purifying SCF1 proteins from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments, protein refolding steps can be used as necessary, in completing configuration of the mature protein. In still other embodiments, high performance liquid chromatography (HPLC) can be employed for final purification steps.

The present disclosure further provides polynucleotides having the coding sequence (e.g., portions of SEQ ID NOs: 2, 3, 15, 17, or 18, or sub-portions thereof) fused in frame to a marker sequence which allows for purification polypeptides. A non-limiting example of a marker sequence is a hexahistidine tag which may be supplied by a vector, preferably a pQE 9 vector, which provides for purification of the polypeptide fused to the marker in the case of a bacterial host, or, for example, the marker sequence may be a hemagglutinin (HA) tag when a mammalian host (e.g., COS 7 cells) is used. The HA tag corresponds to an epitope derived from the influenza hemagglutinin protein (Wilson et al., Cell, 37:767 [1984]).

In certain embodiments, the SCF1 peptides (e.g., SEQ ID NOs: 1, 4-14, and 16) are conservatively modified. Conservatively modified variants applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a conservatively modified variant where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Disclosed herein are polynucleotides encoding at least of SCF1 and compositions thereof. In some embodiments, the polynucleotide is DNA. In some embodiments, the polynucleotide is RNA. In particular embodiments, the polynucleotide is a mRNA. Thus, the disclosure further provides a mRNA comprising an open reading frame encoding all or at least a portion of SCF1 (e.g., immunogenic portion), and compositions thereof.

In some embodiments, the polynucleotides comprise a nucleotide sequence having at least 70% (e.g., 70 . . . 80 . . . 90 . . . 99%) sequence identity to SEQ ID NOs: 2, 3, 15, 17, or 18, or a complement or reverse complement thereof, wherein T may be replaced by U.

The disclosure also provides polynucleotide segments (e.g., of SEQ ID NOs: 2, 3, 15, 17, or 18 or sub-portions thereof) encoding the polynucleotide or mRNA, vectors containing these segments, and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence. In some embodiments, the polynucleotide is optimized for enhanced expression, productive co-translational protein folding, increased stability, or a combination thereof.

In some embodiments, the polynucleotide or mRNA is codon optimized. Codon optimization methods are known in the art and may be used as provided herein. Codon optimization, in some embodiments, may be used to match codon frequencies in target (e.g., in a human) and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide.

In some embodiments, a codon optimized sequence shares less than 95% sequence identity, less than 90% sequence identity, less than 85% sequence identity, less than 80% sequence identity, or less than 75% sequence identity to a naturally occurring or wild-type sequence (e.g., a naturally-occurring or wild-type sequence encoding SCF1).

The polynucleotide or mRNA may further comprise other structures and sequences necessary for proper functionality or stability, including but not limited to a 5′ untranslated region (UTR), a 5′ cap, a 3′ UTR, a 3′ tailing sequence, or any combination thereof. Thus, in some embodiments, the disclosure provides a mRNA comprising: a 5′ UTR, an open reading frame encoding at least a portion of SCF1, and a 3′ UTR.

The polynucleotide or mRNA may comprise a 5′ and 3′ untranslated region (UTR). Sequence elements within the UTRs affect translational efficiency and RNA stability but do not encode a polypeptide. The 3′ UTR relates to the region located at the 3′ end of the mRNA, downstream of the termination codon of a protein-encoding region, which is transcribed but not translated into an amino acid sequence. The 5′UTR refers to the region directly upstream from the initiation codon. Eukaryotic 5′ UTRs contain the Kozak consensus sequence (ACCAUGG), which contains the initiation codon. The eukaryotic 5′ UTR may also contain cis-acting regulatory elements called upstream open reading frames (uORFs) and upstream AUGs (uAUGs) and termination codons, which can impact translation regulation. In some embodiments, the 5′ UTR, 3′ UTR, or both is heterologous to the sequence or open read frame encoding at least a portion of SCF1.

A 5′-cap refers to the structure found on the 5′-end of an mRNA which ordinarily is composed of a guanosine nucleotide connected to the mRNA via a 5′ to 5′ triphosphate linkage. The guanosine may be methylated at the 7-position creating a 7-methylguanosine cap (m7G). In some embodiments, the 5′ cap may be a naturally occurring 5′ cap. In some embodiments, the 5′ cap is a 5′ cap analog or a modified 5′ cap structure which is non-naturally occurring (e.g., phosphorothioate-cap-analogs).

The polynucleotides or mRNAs herein may comprise a 3′ tailing sequence. The 3′ tailing sequence comprises a polyA tail, a polyG quartet, a stem loop sequence, a triple helix forming sequence, a tRNA-like sequence, or any combination thereof. The 3′ poly(A) sequence of mRNA is important for nuclear export, RNA stability and translational efficiency of eukaryotic messenger RNA (mRNA). The poly(A) tail is a segment of RNA at the 3′ end of the molecule that has only adenine bases. For example, a poly(A) tail may contain 10, 20, 30, 40, 50, 60, 70, 80, 90, 100, 110, 120,130, 140, 150, 160, 170, 180, 190, 200, 210, 220, 230, 240, 250, 260, 270, 280, 290 or 300 adenosine monophosphates The poly(A) tail may contain two segments of only adenine bases separated by a linker.

In some embodiments, the polynucleotides or mRNAs herein comprise at least one chemical modification or chemically modified base, nucleoside, or nucleotide. The chemical modifications may comprise any modification which is not naturally present in said RNA or any naturally occurring modification of adenosine (A), guanosine (G), uridine (U), or cytidine (C) ribonucleosides. For example, a single polynucleotide or mRNA may include both naturally occurring and non-naturally-occurring modifications. Chemical modifications may be located in any portion of the polynucleotide or mRNA molecule and the polynucleotide or mRNA molecule may contain any percentage of modified nucleosides (1-100%, such as at least 20% . . . at least 40% . . . or at least 60%). In some embodiments, every particular base or nucleoside may be modified (e.g., every uridine is a modified uridine). In some embodiments, at least 20%, or 50%, or 80% of any single nucleotide (e.g., uracil) in the of the polynucleotide or mRNA is chemically modified. In some embodiments, a particular modification is used for every particular type of nucleoside or base (e.g., every uridine is modified to a 1-methyl-pseudouridine). Exemplary RNA modifications can be found in the RNA modification database (See, mods(dot)rna(dot)albany(dot)edu/home).

In some embodiments, at least one chemical modification comprises a modified uridine residue. Exemplary modified uridine residues include, but are not limited to, pseudouridine, 1-methylpseudouridine, 1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine.

In some embodiments, at least one chemical modification comprises a modified cytosine residue. Exemplary nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-aracytidine, 2′-F-cytidine, and 2′-OH-aracytidine.

In some embodiments, at least one chemical modification comprises a modified adenine residue. Exemplary nucleosides having a modified adenine include 2-amino-purine, 2,6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl)adenosine, 2-methylthio-N6-(cis-hydroxyisopentenyl)adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynoryalylcarbamoyl-adenosine, 2-methylthio-N6-hydroxynoryalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, at least one chemical modification comprises a modified guanine residue. Exemplary nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, and 2′-O-ribosylguanosine (phosphate).

The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995). The compositions may be formulated for any particular mode of administration including for example, systemic administration, oral, rectal, nasal, sublingual, buccal, implants, or parenteral.

### II. Vaccines or Medicaments

The compositions or polynucleotides (e.g., mRNA) described herein may be used to prepare vaccines or another medicament (e.g., for treating or preventing skin infection and/or systemic infections with Candida auris based on inducing an immune response against at least a portion of Surface Colonization Factor (SCF1) from Candida auris (e.g., SEQ ID NO:16 or 19).

The vaccine and medicament may comprise an adjuvant or immunostimulant, or a polynucleotide encoding an adjuvant or immunostimulant (e.g., an adjuvantive polypeptide). Adjuvants and immunostimulants are compounds or compositions that either directly or indirectly stimulate the immune system's response to a co-administered antigen. In some embodiments, the vaccines are not adjuvanted or are self-adjuvanting.

Suitable adjuvants are commercially available as, for example, Glucopyranosyl Lipid Adjuvant (GLA); Pam3CSK4; Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO4)2, AlNa(SO4)2, AlNH4(SO4), and Al(OH)3; salts of calcium (e.g., Ca3(PO4)2), iron or zinc; an insoluble suspension of acylated tyrosine; acylated sugars; cationically or anionically derivatized polysaccharides; polynucleotides (for example, poly IC, poly AU acids, and CpG oligodeoxynucleotides (e.g., Class A or B)); polyphosphazenes; cyanoacrylates; polymerase-(DL-lactide-co-glycoside); bovine serum albumin; diphtheria toxoid; tetanus toxoid; edestin; keyhole-limpet hemocyanin; Pseudomonal Toxin A; choleragenoid; cholera toxin; pertussis toxin; viral proteins; Quil A; aminoalkyl glucosamine phosphate compounds. In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule.

Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166: 1365-1374 (1969)) or glycolipids, lipids or carbohydrates.

In some embodiments, the adjuvantive polypeptide comprises immune activator proteins, such as CD70, CD40 ligand, and constitutively active TLR4, or polycationic peptides (e.g., protamine). In some embodiments, the adjuvantive polypeptide is a flagellin polypeptide. Commercially available mRNA encoding adjuvantive polypeptides are available, for example, as TriMix (See Bonehill, A. et al. Mol. Ther. 16, 1170-1180 (2008), incorporated herein by reference). In some embodiments, the vaccine may comprise at least two separate polynucleotides, one encoding at least a portion of SCF1 and the other encoding an adjuvantive polypeptide (e.g., a flagellin polypeptide or immune activator protein).

The compositions may comprise a delivery vehicle. Exemplary delivery vehicles include, but are not limited to, microparticle compositions comprising poly(lactic acid) (PLA) and/or poly(lactic-co-glycolic acid) (PLGA), albumin nanoparticles, and liposomal compositions. The vaccines may further comprise a delivery vehicle. In some embodiments, the vaccine comprises a lipid nanoparticle encapsulating the disclosed compositions, polynucleotides, or mRNA.

Lipid nanoparticle compositions of the disclosure may include one or more cationic and/or ionizable lipids, phospholipids, neutral or non-cationic lipids, polyethylene glycol (PEG)-lipid conjugates, and/or sterols. In some embodiments, the lipid nanoparticle comprises a cationic lipid and/or ionizable lipid, a neutral or non-cationic lipid, and cholesterol. Cationic and/or ionizable lipids include, for example, amine-containing lipids that can be readily protonated and may have a positive or partial positive charge at physiological pH due to a pKa value between pH 5 and 8. The polar headgroup of the cationic lipids preferably comprises amine derivatives such as primary, secondary, and/or tertiary amines, quaternary ammonium, various combinations of amines, amidinium salts, or guanidine and/or imidazole groups as well as pyridinium, piperazine and amino acid headgroups such as lysine, arginine, ornithine and/or tryptophan. Cationic lipids include, but are not limited to, 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradecoxy)propyl-(2-hydroxyethyl)-dimethylazanium bromide (DMRIE), didodecyl(dimethyl)ammonium bromide (DDAB), 1,2-dioleyloxypropyl-3-dimethyl-hydroxyethyl ammonium bromide (DORIE), 3β-[N—(N\N′-dimethylamino-ethane)carbamoyl]cholesterol (DC-Chol) or dioleyl ether phosphatidylcholine (DOEPC). Ionizable lipids include, but are not limited to, 1,2-dioleyloxy-3-dimethylamino-propane (DODMA).

In some embodiments, the lipid nanoparticle comprises a polyethylene glycol (PEG)-lipid conjugate. A PEG-lipid conjugate may include, but is not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-DMG (1,2-dimyristoyl-rac-glycero-3-methoxypolyethylene glycol), PEG-c-DOMG (R-3-|(ω-methoxy poly(ethylene glycol)2000)carbamoyl)]-1,2-dimyristyloxlpropyl-3-amine), PEG-DMA (PEG-dimethacrylate), PEG-DLPE (1,2-didodecanoyl-sn-glycero-3-phosphoethanolamine-PEG), PEG-DMPE (PEG-1,2-dimyristoyl-sn-glycero-3-phosphoethanolamine), PEG-DPPC (PEG-dipalmitoyl phosphatidylcholine), PEG-N,N-di(tetradecyl)acetamide, or a PEG-DSPE (1, 2-distearoyl-sn-glycero-3-phosphoethanolamine-poly(ethylene glycol)) lipid. In some embodiments, the lipid nanoparticle comprises PEG-DMG and/or PEG-N,N-di(tetradecyl)acetamide.

The sterol may comprise cholesterol, fecosterol, ergosterol, campesterol, sitosterol, stigmasterol, brassicasterol or a sterol ester, such as cholesteryl hemisuccinate, cholesteryl sulfate, or any other derivatives of cholesterol. A neutral or non-cationic lipid may include one or more phospholipids. Phospholipids include a phospholipid moiety and one or more fatty acid moieties. A phospholipid moiety may include, but is not limited to, phosphatidyl choline, phosphatidyl ethanolamine, phosphatidyl glycerol, phosphatidyl serine, phosphatidic acid, 2-lysophosphatidyl choline, and sphingomyelin. A fatty acid moiety may include, but is not limited to, lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid.

Phospholipids suitable for use in the compositions may include, but are not limited to, phosphatidylglycerol (PG) including dimyristoyl phosphatidylglycerol (DMPG) and 1,2-dioleoyl-sn-glycero-3-phospho-rac-(1-glycerol) sodium salt (DOPG); phosphatidylcholine (PC), including egg yolk phosphatidylcholine, dimyristoyl phosphatidylcholine (DMPC), 1,2-distearoyl-sn-glycero-3-phosphocholine (DSPC), 1,2-dilinoleoyl-sn-glycero-3-phosphocholine (DLPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-didocosahexaenoyl-sn-glycero-3-phosphocholine, 1,2-diarachidonoyl-sn-glycero-3-phosphocholine; phosphatidylethanolamine (PE) including 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine; phosphatidic acid (PA); phosphatidylinositol (PI); phosphatidylserine (PS); and sphingomyelin (SM).

The positively charged lipid structures described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

The vaccine or medicament of the present disclosure may also contain other compounds, which may be biologically active or inactive. For example, one or more immunogenic and antigenic portions of polypeptides or polynucleotides encoding immunogenic polypeptides, or nucleic acid(s) encoding thereof, may be present within the vaccine. The vaccine or medicament may generally be used for prophylactic and therapeutic purposes.

The vaccines or medicaments may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, or via inhalation.

The vaccines or medicaments may also comprise buffers (e.g., neutral buffered saline or phosphate buffered saline), carbohydrates (e.g., glucose, mannose, sucrose or dextrans), mannitol, proteins, polypeptides, or amino acids such as glycine, antioxidants, bacteriostats, chelating agents such as EDTA or glutathione, solutes that render the formulation isotonic, hypotonic, or weakly hypertonic with the blood of a recipient, suspending agents, thickening agents and/or preservatives. Alternatively, vaccines or medicaments may be formulated as a lyophilisate. The compositions and vaccines may be prepared, packaged, or sold in a form suitable for bolus administration or sold in unit dosage forms, such as in ampules or multi-dose containers. In some embodiments, the compositions and vaccines contain a preservative.

### III. Exemplary Methods

The present disclosure provides methods for treating, reducing, or preventing skin infection or systemic infection with Candida auris based on inducing an immune response against at least a portion of Surface Colonization Factor (SCF1) from Candida auris (e.g., SEQ ID NO:16). Generally, the immune response can include a humoral immune response, a cell-mediated immune response, or both. A humoral response can be determined by a standard immunoassay for antibody levels in a serum sample from the subject receiving the composition or polypeptide. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of the composition or polypeptide. Following incubation of e.g., PBMCs with a stimulator for an appropriate period, [3H]thymidine incorporation can be determined. The subset of T cells that is proliferating can be determined using flow cytometry.

The methods herein include administering to the subject an effective amount of the compositions, polynucleotides, mRNA, vaccines, or medicaments disclosed herein. In some embodiments, the subject is a human. An “effective amount” is an amount that is delivered to a subject, either in a single dose or as part of a series, which is effective for inducing an immune response in the subject. This amount varies depending upon the health and physical condition of the subject to be treated, the capacity of the subject's immune system to synthesize antibodies, the formulation of the compositions, and other relevant factors. It is expected that the amount will fall in a relatively broad range that can be determined by one of skill in the art through routine trials.

The compositions, peptides, polynucleotides, mRNA, vaccines, or medicaments disclosed herein can be administered in a wide variety of therapeutic dosage forms. The route and regimen of administration will vary depending upon the population and the indication for vaccination and is to be determined by the skilled practitioner. For example, the compositions, vaccines, or medicaments disclosed herein may be administered parentally, e.g., intravenous (either by bolus or infusion methods), intraperitoneal, subcutaneous, topical with or without occlusion, or intramuscular form.

The administration may comprise an initial immunization or dose and at least one subsequent immunization or booster dose, following known standard immunization protocols. The boosting doses will be adequately spaced at such times where the levels of circulating antibody fall below a desired level. Boosting doses may consist of the compositions or vaccines disclosed herein and may comprise alternative carriers and/or adjuvants. The booster dosage levels may be the same or different that those of the initial immunization dosage.

The specific dose level may depend upon a variety of factors including the activity of the polynucleotides (e.g., mRNA), composition, peptides, vaccine, or medicament, the age, body weight, general health, and diet of the subject, time of administration, and route of administration. For prophylaxis purposes, the amount of the polynucleotide(s) (e.g., mRNA) or peptides in each dose is an amount which induces an immunoprotective response without significant adverse side effects.

A wide range of second therapies may be used in conjunction with the polynucleotides, peptides, compositions, vaccines, medicaments, and methods of the present disclosure. The second therapy may be administration of an anti-fungal agent that treats Candida auris infection. The second therapy may be administered at the same time as the initial therapy. The second therapy may be administered in the same or composition or in a separate composition administered at substantially the same time as the initial therapy. In some embodiments, the second therapy may precede or follow administration of the disclosed polynucleotides, compositions, vaccines, medicaments, and methods by time intervals ranging from hours to weeks.

### IV. Systems and Kits

Also disclosed herein are systems or kits comprising the compositions, peptides, polynucleotides, vaccines, or medicaments disclosed herein. The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed peptide and/or product and another agent (e.g., an anti-fungal agent, a pain reliever, etc.) for delivery to a patient or a cell.

The kits can also comprise instructions for using the components of the kit. The instructions are relevant materials or methodologies pertaining to the kit. The materials may include any combination of the following: background information, list of components, brief or detailed protocols for using the compositions, troubleshooting, references, technical support, and any other related documents. Instructions can be supplied with the kit or as a separate member component, either as a paper form or an electronic form which may be supplied on computer readable memory device or downloaded from an internet website, or as recorded presentation. It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks). In some embodiments, the kits comprise a delivery device or container. In some embodiments, the delivery device or container comprises a syringe or syringe vial. In some embodiments, the delivery device or container is pre-filled with the composition. The kits provided herein may be in suitable packaging. Suitable packaging includes, but is not limited to, vials, bottles, jars, flexible packaging, and the like. Individual member components of the kits may be physically packaged together or separately.

### V. Anti-SCF1 Antibody Generation

In some embodiments, anti-SCF1 antibodies are used for treatment of Candida auris infection. The antibodies may be prepared using various immunogens. In one embodiment, the immunogen is a SCF1 peptide (e.g., as shown in SEQ ID NOs:1, 4-14, and 16), or portions thereof, or an expression vector configured to express such SCF1 peptides, to generate antibodies that recognize SCF1. Such antibodies include, but are not limited to polyclonal, monoclonal, chimeric, single chain, Fab fragments, and Fab expression libraries.

Various procedures known in the art may be used for the production of polyclonal antibodies directed against SCF1 peptides. For the production of antibody, various host animals can be immunized by injection with the peptide corresponding to a SCF1 epitope including but not limited to rabbits, mice, rats, sheep, goats, etc. In certain embodiments, the SCF1 peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette Guerin) and Corynebacterium parvum).

For preparation of monoclonal antibodies directed toward SCF1, it is contemplated, in certain embodiments, that any technique that provides for the production of antibody molecules by continuous cell lines in culture will find use with the present disclosure (See e.g., Harlow and Lane, Antibodies: A Laboratory Manual, Cold Spring Harbor Laboratory Press, Cold Spring Harbor, NY). These include but are not limited to the hybridoma technique originally developed by Köhler and Milstein (Köhler and Milstein, Nature 256:495 497 [1975]), as well as the trioma technique, the human B cell hybridoma technique (See e.g., Kozbor et al., Immunol. Tod., 4:72 [1983]), and the EBV hybridoma technique to produce human monoclonal antibodies (Cole et al., in Monoclonal Antibodies and Cancer Therapy, Alan R. Liss, Inc., pp. 77 96 [1985]).

In certain embodiments, monoclonal antibodies are produced in germ free animals utilizing technology such as that described in PCT/US90/02545. Furthermore, it is contemplated that human antibodies may be generated by human hybridomas (Cote et al., Proc. Natl. Acad. Sci. USA 80:2026 2030 [1983]) or by transforming human B cells with EBV virus in vitro (Cole et al., in Monoclonal Antibodies and Cancer Therapy, Alan R. Liss, pp. 77 96 [1985]).

In addition, it is contemplated that techniques described for the production of single chain antibodies (U.S. Pat. No. 4,946,778; herein incorporated by reference) will find use in producing SCF1 specific single chain antibodies. An additional embodiment of the invention utilizes the techniques described for the construction of Fab expression libraries (Huse et al., Science 246:1275 1281 [1989]) to allow rapid and easy identification of monoclonal Fab fragments with the desired specificity for SCF1.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. As is well known in the art, the immunogenic peptide should generally be provided free of the carrier molecule used in any immunization protocol. For example, if the peptide was conjugated to KLH, it may be conjugated to BSA, or used directly, in a screening assay to detect SCF1 in a sample. The foregoing antibodies can be used to detect HPgV-2 in a biological sample from an individual (e.g., suspected of being infected with HPgV-2). The biological sample can be a biological fluid, such as, but not limited to, blood, serum, plasma, interstitial fluid, urine, cerebrospinal fluid, and the like, containing cells.

### EXAMPLES

### Example 1

The Candida auris-specific adhesin SCF1 governs surface association and colonization Candida auris is an emerging fungal pathogen responsible for healthcare-associated outbreaks driven by persistent surface colonization. We functionally characterized the arsenal of adhesins utilized by C. auris for surface association and discovered that Surface Colonization Factor (SCF1), an adhesin unique to C. auris, and IFF4109, a conserved IFF/HYR family adhesin, are major drivers of attachment and are critical for colonization of biotic and abiotic substrates. Transcriptional control of SCF1 varied widely among C. auris isolates and was positively associated with adhesive plasticity across all five clades. Unlike canonical adhesins which mediate surface attachment via hydrophobic interactions, SCF1 relies on cation-substrate interactions in a manner analogous to barnacle and mussel adhesion proteins. These findings explain the ability of C. auris to colonize biotic and abiotic surfaces, a trait critical for its pathogenesis.

### Materials and Methods

### Strains and Culture Conditions

A list of all strains used in this study is included in Table 1.

TABLE 1

| Strain | | | |
|---|---|---|---|
| Name | Alias | Genotype | Source |
| CauTO33 | C. auris B11109 | AR0382 | (52) |
| CauTO186 | C. auris B11109 | AR0382 | This |
| | | Δals(B9J08_002582)::NAT | Study |
| CauTO187 | C. auris B11109 | AR0382 | This |
| | | Δals (B9J08_004498)::NAT | Study |
| CauTO226 | C. auris B11109 | AR0382 | This |
| | | Δals(B9J08_004112)::NAT | Study |
| CauTO233 | C. auris B11109 | AR0382 | This |
| | | Δiff(B9J08_004100)::NAT | Study |
| CauTO234 | C. auris B11109 | AR0382 | This |
| | | Δiff(B9J08_004109)::NAT | Study |
| CauTO235 | C. auris B11109 | AR0382 | This |
| | | Δiff(B9J08_004098)::NAT | Study |
| CauTO236 | C. auris B11109 | AR0382 | This |
| | | Δiff(B9J08_004110)::NEO | Study |
| CauTO247 | C. auris B11109 | AR0382 | This |
| | | Δiff(B9J08_001531)::NAT | Study |
| CauTO248 | C. auris B11109 | AR0382 | This |
| | | Δiff(B9J08 004892)::NAT | Study |
| CauTO249 | C. auris B11109 | AR0382 | This |
| | | Δiff(B9J08_001155)::NAT | Study |
| CauTO250 | C. auris B11109 | AR0382 | This |
| | | Δiff(B9J08_004451)::NAT | Study |
| CauTO251 | C. auris B11109 | AR0382 | This |
| | | Δiff(B9J08_000675)::NAT | Study |
| At pTO131 | Agrobacterium | EHA105 harboring pTO128 | (50) |
| | tumefaciens | (pPZP-NAT) | |
| | EHA105 | | |
| CauTO219 | C. auris B11109 | AR0382 | This |
| | | tnSWIl(B9J08_003460) | Study |
| CauTO322 | C. auris B11109 | AR0382 | This |
| | | tnBCY1(B9J08_002818) | Study |
| CauTO261 | C. auris B11109 | AR0382 | This |
| | | Δscf1(B9J08_001458)::NAT | Study |
| CauTO320 | C. auris B11109 | AR0382 | This |
| | | Δscf1(B9J08_001458)::NAT + | Study |
| | | NAT::SCF1-FLAG NEO | |
| CauTO307 | C. auris B11109 | AR0382 | This |
| | | Δiff(B9J08_004109)::NAT + | Study |
| | | IFF(B9J08_004109) NEO | |
| CauTO270 | C. auris B11109 | AR0382 | This |
| | | Δscf1(B9J08_001458)::NAT | Study |
| | | Δiff(B9J08_004109)::NEO | |
| CaTO1 | C. albicans | SC5314 | |
| | SC5314 | | |
| CaTO227 | C. albicans | SC5314 als1::FRT/als1::FRT | This |
| | SC5314 | | Study |
| CauTO436 | C. auris B11109 | AR0382 pTEF1-ALS4112 NAT | This |
| | | | Study |
| CauTO32 | C. auris B11220 | AR0381 | (50) |
| CauTO34 | C. auris B11221 | AR0383 | (50) |
| CauTO35 | C. auris B11222 | AR0384 | (50) |
| CauTO36 | C. auris B11244 | AR0385 | (50) |
| CauTO37 | C. auris B11245 | AR0386 | (50) |
| CauTO38 | C. auris B8441 | AR0387 | (50) |
| CauTO39 | C. auris B11098 | AR0388 | (50) |
| CauTO40 | C. auris B11203 | AR0389 | (50) |
| CauTO41 | C. auris B11205 | AR0390 | (50) |
| CauTO52 | C. auris B11243 | AR0931 | (50) |
| CauTO53 | C. auris | AR1097 | (50) |
| | IFRC2087 | | |
| CauTO325 | C. auris B14308 | AR1099 | (50) |
| CauTO326 | C. auris B13463 | AR1100 | (50) |
| CauTO327 | C. auris B18578 | AR1101 | (50) |
| CauTO328 | C. auris B17835 | AR1102 | (50) |
| CauTO329 | C. auris B18683 | AR1103 | (50) |
| CauTO330 | C. auris B18017 | AR1104 | (50) |
| CauTO331 | C. auris B11842 | AR1105 | (73) |
| CauTO315 | C. auris | Chicago-1 | (73) |
| | A.04.TO.Ax.9- | | |
| | ORG1 | | |
| CauTO316 | C. auris | Chicago-2 | (73) |
| | A.04.TO.Ax.9- | | |
| | ORG2 | | |
| CauTO317 | C. auris | Chicago-3 | (73) |
| | B.02.TO.lc.9- | | |
| | ORG1 | | |
| CauTO318 | C. auris | Chicago-4 | (50) |
| | B.06.TO.Ax.9- | | |
| | ORG1 | | |
| CaTO380 | C. albicans | P75010 | ATCC |
| | P75010 | | |
| CaTO382 | C. albicans 12C | 12C | ATCC |
| CaTO381 | C. albicans 19F | 19F | ATCC |
| CaTO388 | C. albicans L26 | L26 | ATCC |
| CaTO391 | C. albicans | P37005 | ATCC |
| | P37005 | | |
| CaTO383 | C. albicans | P37037 | ATCC |
| | P37037 | | |
| CaTO392 | C. albicans | P37039 | ATCC |
| | P37039 | | |
| CaTO384 | C. albicans | P78048 | ATCC |
| | P78048 | | |
| CaTO377 | C. albicans | P94015 | ATCC |
| | P94015 | | |
| CaTO379 | C. albicans | P76055 | ATCC |
| | P76055 | | |
| CaTO389 | C. albicans | P76067 | ATCC |
| | P76067 | | |
| CaTO378 | C. albicans | P34048 | ATCC |
| | P34048 | | |
| CaTO386 | C. albicans | P57055 | ATCC |
| | P57055 | | |
| CaTO390 | C. albicans | P78042 | ATCC |
| | P78042 | | |
| CaTO375 | C. albicans | GC75 | ATCC |
| | GC75 | | |
| CaTO376 | C. albicans | P600002 | ATCC |
| | P600002 | | |
| CaTO385 | C. albicans | P75016 | ATCC |
| | P75016 | | |
| CaTO387 | C. albicans | P75063 | ATCC |
| | P75063 | | |
| CaTO393 | C. albicans P87 | P87 | ATCC |
| ChTO46 | C. haemulonii | AR0395 | (50) |
| | B10441 | | |
| ChTO346 | C. haemulonii | AR0395 | This |
| | B10441 | Δscf1(CXQ85_003100)::NAT | Study |
| CauTO306 | C. auris B11109 | AR0382 | This |
| | | Δscf1(B9J08_001458)::NAT + | Study |
| | | NAT::SCF1 NEO | |
| CauTO364 | C. auris B11109 | AR0382 | This |
| | | Δscf1(B9J08_001458)::NAT + | Study |
| | | NAT::SCF1_{Ch} | |
| | | (CXQ85_003100) NEO | |
| ChTO324 | C. haemulonii | AR0932 | (50) |
| ChTO44 | C. haemulonii | AR0393 | (52) |
| CauTO312 | C. auris B11220 | AR0381 pTEFI-SCF1_{AR0381} | This |
| | | NAT | Study |
| CauTO323 | C. auris B11220 | AR0381 pTEF1-SCF1_{AR0382} | This |
| | | NAT | Study |
| CauTO438 | C. auris B11220 | AR0382 | This |
| | | Δscf1(B9J08_001458)::NAT + | Study |
| | | NAT::SCF1_{AR0381} | |
| | | (CJI96_0001187) NEO | |
| CauTO308 | C. auris B8441 | AR0387 pTEF1-SCF1 NAT | This |
| | | | Study |
| CauTO432 | C. auris B8441 | AR0382 | This |
| | | Δscf1(B9J08_001458)::NAT + | Study |
| | | NAT::SCF1-FLAG_{R178A K181A} | |
| | | NEO | |
| CauTO433 | C. auris B8441 | AR0382 | This |
| | | Δscf1(B9J08_001458)::NAT + | Study |
| | | NAT::SCF1-FLAG_{K163A} NEO | |
| CauTO434 | C. auris B8441 | AR0382 | This |
| | | Δscf1(B9J08_001458)::NAT + | Study |
| | | NAT::SCF1-FLAG_{K147A R148A} | |
| | | _{K150A K152A} NEO | |
| CauTO430 | C. auris B8441 | AR0382 | This |
| | | Δscf1(B9J08_001458):: NAT + | Study |
| | | NAT::SCF1-FLAG_{R54A R55A} | |
| | | NEO | |
| CauTO453 | C. auris B8441 | AR0382 | This |
| | | Δscf1(B9J08_001458)::NAT + | Study |
| | | NAT::SCF1-FLAG_{H52A H53A R54A} | |
| | | _{R55A} NEO | |
| CauTO455 | C. auris B8441 | AR0382 | This |
| | | Δscf1(B9J08_001458)::NAT + | Study |
| | | NAT::SCF1-FLAG_{K44A H55A Y46A} | |
| | | _{W47A K49A} NEO | |
| CauTO33 | C. auris B11109 | AR0382 | (52) |
| CauTO186 | C. auris B11109 | AR0382 | This |
| | | Δals(B9J08 002582)::NAT | Study |
| CauTO187 | C. auris B11109 | AR0382 | This |
| | | Δals(B9J08_004498)::NAT | Study |
| CauTO226 | C. auris B11109 | AR0382 | This |
| | | Δals(B9J08_004112)::NAT | Study |
| CauTO233 | C. auris B11109 | AR0382 | This |
| | | Δiff(B9J08_004100)::NAT | Study |
| CauTO234 | C. auris B11109 | AR0382 | This |
| | | Δiff(B9J08_004109)::NAT | Study |
| CauTO235 | C. auris B11109 | AR0382 | This |
| | | Δiff(B9J08_004098)::NAT | Study |
| CauTO236 | C. auris B11109 | AR0382 | This |
| | | Δiff(B9J08_004110)::NEO | Study |
| CauTO247 | C. auris B11109 | AR0382 | This |
| | | Δiff(B9J08_001531)::NAT | Study |
| CauTO248 | C. auris B11109 | AR0382 | This |
| | | Δiff(B9J08_004892)::NAT | Study |
| CauTO249 | C. auris B11109 | AR0382 | This |
| | | Δiff(B9J08_001155)::NAT | Study |
| CauTO250 | C. auris B11109 | AR0382 | This |
| | | Δiff(B9J08_004451)::NAT | Study |
| CauTO251 | C. auris B11109 | AR0382 | This |
| | | Δiff(B9J08_000675)::NAT | Study |
| At pTO131 | Agrobacterium tumefaciens | EHA 105 harboring pTO128 | (50) |
| | EHA105 | (pPZP-NAT) | |
| CauTO219 | C. auris B11109 | AR0382 | This |
| | | tnSWIl(B9J08_003460) | Study |
| CauTO322 | C. auris B11109 | AR0382 | This |
| | | tnBCY1(B9J08_002818) | Study |
| CauTO261 | C. auris B11109 | AR0382 | This |
| | | Δscf1(B9J08_001458)::NAT | Study |
| CauTO320 | C. auris B11109 | AR0382 | This |
| | | Δscf1(B9J08_001458)::NAT + | Study |
| | | NAT::SCF1-FLAG NEO | |
| CauTO307 | C. auris B11109 | AR0382 | This |
| | | Δiff(B9J08_004109)::NAT + | Study |
| | | IFF(B9J08_004109) NEO | |
| CauTO270 | C. auris B11109 | AR0382 | This |
| | | Δscf1(B9J08_001458)::NAT | Study |
| | | Δiff(B9J08_004109)::NEO | |
| CaTO1 | C. albicans SC5314 | SC5314 | |
| CaTO227 | C. albicans SC5314 | SC5314 | This |
| | | als1::FRT/als1::FRT | Study |
| CauTO436 | C. auris B11109 | AR0382 pTEF1-ALS4112 | This |
| | | NAT | Study |
| CauTO32 | C. auris B11220 | AR0381 | (50) |
| CauTO34 | C. auris B11221 | AR0383 | (50) |
| CauTO35 | C. auris B11222 | AR0384 | (50) |
| CauTO36 | C. auris B11244 | AR0385 | (50) |
| CauTO37 | C. auris B11245 | AR0386 | (50) |
| CauTO38 | C. auris B8441 | AR0387 | (50) |
| CauTO39 | C. auris B11098 | AR0388 | (50) |
| CauTO40 | C. auris B11203 | AR0389 | (50) |
| CauTO41 | C. auris B11205 | AR0390 | (50) |
| CauTO52 | C. auris B11243 | AR0931 | (50) |
| CauTO53 | C. auris IFRC2087 | AR1097 | (50) |
| CauTO325 | C. auris B14308 | AR1099 | (50) |
| CauTO326 | C. auris B13463 | AR1100 | (50) |
| CauTO327 | C. auris B18578 | AR1101 | (50) |
| CauTO328 | C. auris B17835 | AR1102 | (50) |
| CauTO329 | C. auris B18683 | AR1103 | (50) |
| CauTO330 | C. auris B18017 | AR1104 | (50) |
| CauTO331 | C. auris B11842 | AR1105 | (73) |
| CauTO315 | C. auris A.04.TO.Ax.9- | Chicago-1 | (73) |
| | ORG1 | | |
| CauTO316 | C. auris A.04.TO.Ax.9- | Chicago-2 | (73) |
| | ORG2 | | |
| CauTO317 | C. auris B.02.TO.Ic.9- | Chicago-3 | (73) |
| | ORG1 | | |
| CauTO318 | C. auris B.06.TO.Ax.9- | Chicago-4 | (50) |
| | ORG1 | | |
| CaTO380 | C. albicans P75010 | P75010 | ATCC |
| CaTO382 | C. albicans 12C | 12C | ATCC |
| CaTO381 | C. albicans 19F | 19F | ATCC |
| CaTO388 | C. albicans L26 | L26 | ATCC |
| CaTO391 | C. albicans P37005 | P37005 | ATCC |
| CaTO383 | C. albicans P37037 | P37037 | ATCC |
| CaTO392 | C. albicans P37039 | P37039 | ATCC |
| CaTO384 | C. albicans P78048 | P78048 | ATCC |
| CaTO377 | C. albicans P94015 | P94015 | ATCC |
| CaTO379 | C. albicans P76055 | P76055 | ATCC |
| CaTO389 | C. albicans P76067 | P76067 | ATCC |
| CaTO378 | C. albicans P34048 | P34048 | ATCC |
| CaTO386 | C. albicans P57055 | P57055 | ATCC |
| CaTO390 | C. albicans P78042 | P78042 | ATCC |
| CaTO375 | C. albicans GC75 | GC75 | ATCC |
| CaTO376 | C. albicans P600002 | P600002 | ATCC |
| CaTO385 | C. albicans P75016 | P75016 | ATCC |
| CaTO387 | C. albicans P75063 | P75063 | ATCC |
| CaTO393 | C. albicans P87 | P87 | ATCC |
| ChTO46 | C. haemulonii B10441 | AR0395 | (50) |
| ChTO346 | C. haemulonii B10441 | AR0395 | This |
| | | Δscf1(CXQ85_003100)::NAT | Study |
| CauTO306 | C. auris B11109 | AR0382 | This |
| | | Δscf1(B9J08_001458)::NAT + | Study |
| | | NAT::SCF1 NEO | |
| CauTO364 | C. auris B11109 | AR0382 | This |
| | | Δscf1(B9J08_001458)::NAT + | Study |
| | | NAT::SCF1_{Ch} | |
| | | (CXQ85_003100) NEO | |
| ChTO324 | C. haemulonii | AR0932 | (50) |
| ChTO44 | C. haemulonii | AR0393 | (52) |
| CauTO312 | C. auris B11220 | AR0381 pTEF1-SCF1_{AR0381} | This |
| | | NAT | Study |
| CauTO323 | C. auris B11220 | AR0381 pTEF1-SCF1_{AR0382} | This |
| | | NAT | Study |
| CauTO438 | C. auris B11220 | AR0382 | This |
| | | Δscf1(B9J08_001458)::NAT + | Study |
| | | NAT::SCF1_{AR0381} | |
| | | (CJI96_0001187) NEO | |
| CauTO308 | C. auris B8441 | AR0387 pTEF1-SCF1 NAT | This |
| | | | Study |
| CauTO432 | C. auris B8441 | AR0382 | This |
| | | Δscf1(B9J08_001458)::NAT + | Study |
| | | NAT::SCF1-FLAG_{R178A} | |
| | | _{K181A} NEO | |
| CauTO433 | C. auris B8441 | AR0382 | This |
| | | Δscf1(B9J08_001458)::NAT + | Study |
| | | NAT::SCF1-FLAG_{K163A} | |
| | | NEO | |
| CauTO434 | C. auris B8441 | AR0382 | This |
| | | Δscf1(B9J08_001458)::NAT + | Study |
| | | NAT::SCF1-FLAG_{K147A} | |
| | | _{R148A K150A K152A} NEO | |
| CauTO430 | C. auris B8441 | AR0382 | This |
| | | Δscf1(B9J08_001458)::NAT + | Study |
| | | NAT::SCF1-FLAG_{R54A R55A} | |
| | | NEO | |
| CauTO453 | C. auris B8441 | AR0382 | This |
| | | Δscf1(B9J08_001458)::NAT + | Study |
| | | NAT::SCF1-FLAG_{H52A H53A} | |
| | | _{R54A R55A} NEO | |
| CauTO455 | C. auris B8441 | AR0382 | This |
| | | Δscf1 (B9J08_001458)::NAT + | Study |
| | | NAT::SCF1-FLAG_{K44A H55A} | |
| | | _{Y46A W47A K49A} NEO | |

Clinical C. auris isolates were obtained through the CDC/FDA Antibiotic Resistant Isolate Bank (67) or from Rush University Medical Center, Chicago, IL (Gift from Mary Hayden). Except where specified, C. auris cells were cultured at 30° C. in YPD liquid media (1% yeast extract, 2%7 peptone, 2%7 dextrose) with constant agitation. All strains were maintained as frozen stocks of 25% glycerol at −80° C.

### Genomic DNA Isolation

Genomic DNA was isolated using a PCA extraction method. Briefly, yeast cells were incubated overnight in liquid YPD at 30° C. then harvested by centrifugation and resuspended in lysis buffer (2% (v/v) Triton X-100, 1% (w/v) SDS, 100 mM NaCl, 10 mM Tris-Cl, 1 mM EDTA). The cell suspension was disrupted by bead-beating and released DNA was extracted into PCA and then into Chloroform. The resulting DNA was purified by ethanol precipitation and resuspended in water before being treated with RNase A. RNase was heat-inactivated, then extracted DNA was purified by ethanol precipitation and resuspended in water.

### Primers and Plasmids

A list of all plasmids used in this study is included in Table 2.

TABLE 2

| Name | Description | Source |
|---|---|---|
| pTO135 | pENO1-CaCas9-tCyc1, AMP | (52) |
| pTO136 | pADH1-tRNA-Ala-gRNA-tracrRNA-HDV-tAgTEF2, AMP | (52) |
| pTO137 | RFP-tADH1-pAgTEF2-NAT1-tAGTEF2, AMP | (52) |
| pTO169 | ACE2 NEO, AMP | (52) |
| pTO139 | pUC19, AMP | (51) |
| pTO144 | als(B9J08_002582)::NAT, AMP | This Study |
| pTO167 | als(B9J08_004498)::NAT, AMP | This Study |
| pTO166 | als(B9J08_004112)::NAT, AMP | This Study |
| pTO145 | iff(B9J08_004100)::NAT, AMP | This Study |
| pTO148 | iff(B9J08_004109)::NAT, AMP | This Study |
| pTO202 | iff(B9J08_004098):: NAT, AMP | This Study |
| pTO188 | iff( B9J08_004110)::NEO, AMP | This Study |
| pTO146 | iff(B9J08_001531)::NAT, AMP | This Study |
| pTO147 | iff(B9J08_004892)::NAT, AMP | This Study |
| pTO205 | iff(B9J08_001155)::NAT, AMP | This Study |
| pTO206 | iff(B9J08_004451)::NAT, AMP | This Study |
| pTO207 | iff(B9J08_000675):: NAT, AMP | This Study |
| pTO222 | IFF(B9J08_004109) NEO, AMP | This Study |
| pTO211 | scf1::NAT, AMP | This Study |
| pTO221 | iff( B9J08_004109)::NEO, AMP | This Study |
| pTO100 | pLC49 C. albicans NAT flp | (51) |
| pTO102 | pLC953 C. albicans Cas9 sgRNA | (2) |
| pTO288 | pTEF1-ALS(B9J08_004112), NAT, AMP | This Study |
| pTO255 | scf1(CXQ85_003100)::NAT, AMP | This Study |
| pTO264 | SCF1_{Ch} (CXQ85_003100) NEO | This Study |
| pTO250 | pTEF1-SCF1 NAT, AMP | This Study |
| pTO223 | SCF1 NEO, AMP | This Study |
| pTO284 | SCF1_{AR0381} (CJ196_0001187) NEO, AMP | This Study |
| pTO280 | SCF1-FLAG NEO, AMP | This Study |
| pTO281 | SCF1-FLAG_{K163A} NEO, AMP | This Study |
| pTO282 | SCF1-FLAG_{K147A R14SA K150A KIS2A} NEO, AMP | This Study |
| pTO283 | SCF1-FLAG_{R54A R55A} NEO, AMP | This Study |
| pTO268 | SCF1-FLAG_{R178A K181A} NEO | This Study |
| pTO292 | SCF1-FLAG_{H52A H53A R54A R55A} NEO | This Study |
| pTO293 | SCF1-FLAG_{K44A H55A Y46A W47A K49A} NEO | This Study |

A list of all primers used in this example is included in Table 3.

TABLE 3

| | | |
|---|---|---|
| Name | Sequence | Purpose |

qPCR Primers

| | | |
|---|---|---|
| oTO359 | CGTGCTGTGTTCCCATCCAT (SEQ ID NO: 22) | C. auris ACT1 |
| oTO360 | AGCCTCATCACCGACATACG (SEQ ID NO: 23) | |
| oTO1251 | GTGAGAGTGAGGTCGGAACG (SEQ ID NO: 24) | C. auris SCF1 |
| oTO1252 | CAGCTTCTCCTTCTGGCTCC (SEQ ID NO: 25) | |
| oTO615 | GGGAGACACCTTGACGCTTT (SEQ ID NO: 26) | IFF (B9J08_004109) |
| oTO616 | GTTGGCTCAGGGAAGTCGAA (SEQ ID NO: 27) | |
| oTO1253 | TGAGAGATTTAGAGCCGCCG (SEQ ID NO: 28) | C. haemulonii ACT1 |
| oTO1254 | TACGCTCTGCAATACCTGGG (SEQ ID NO: 28) | |
| oTO1229 | TTGGTGAAGGAGCAACCGAG (SEQ ID NO: 30) | C. haemulonii SCF1 |
| oTO1230 | GGGGCTTCAAGTGTCTGACT (SEQ ID NO: 31) | |

Amplification of transformation cassettes

| | | |
|---|---|---|
| oTO143 | CCTCTTTGTAGTTCAACTTATGC (SEQ ID NO: 32) | Amplification of |
| oTO41 | GTCCCAAAACCTTCTCAAGC (SEQ ID NO: 33) | Cas9 Cassette |
| oTO18 | CAGGAAACAGCTATGAC (SEQ ID NO: 34) | Amplification of |
| oTO19 | GTAAAACGACGGCCAG (SEQ ID NO: 35) | Repair Cassettes |
| oTO1159 | gactacaaggacgacgatgacaagACAACCACCACTACTTCG (SEQ ID NO: 36) | Overlap Extension |
| oTO1160 | cttgtcatcgtcgtccttgtagtcTTCACACTCACACACCCAAC (SEQ ID NO: 37) | PCR for SCF1-FLAG |
| oTO224 | GCTATTACGCCAGCTGG (SEQ ID NO: 38) | Cassette |
| oTO225 | CGCAATTAATGTGAGTTAGC (SEQ ID NO: 39) | |
| oTO945 | CAGTGGAGCGGAAACTC (SEQ ID NO: 40) | |
| oTO946 | GTGGACCTGATTTGACTGG (SEQ ID NO: 41) | |
| oTO947 | CCTGAATCTCTCAACGAAG (SEQ ID NO: 42) | pTO222 Repair |
| oTO948 | GTGGTGTAAGCGATTGTG (SEQ ID NO: 43) | Cassette |
| oTO1161 | GCAAGACGAACCTCCAAC (SEQ ID NO: 44) | pTO250 Repair |
| | | Cassette |

Amplification of plasmid fragments for Gibson Assembly

| | | |
|---|---|---|
| oTO590 | GGATCCTCTAGAGTCGAC (SEQ ID NO: 45) | Amplification of |
| oTO591 | GGTACCGAGCTCGAATTC (SEQ ID NO: 46) | pUC19 backbone |
| oTO668 | ACTGGATGGCGGCGTTAG (SEQ ID NO: 47) | Amplification of |
| oTO669 | CGACATGGAGGCCCAGAATAC (SEQ ID NO: 48) | NEO cassette |
| oTO874 | ATCAAGCTTGCCTCGTCC (SEQ ID NO: 49) | Amplification of |
| | | NAT cassette |
| oTO184 | ttcgagctcggtaccGTACTCATCAGAGTATACGATGC (SEQ ID NO: 50) | Assembly of pTO144 |
| oTO185 | acgccgccatccagtGAGCAGTCGATGCTTCAAATTAAATC (SEQ ID NO: 51) | |
| oTO186 | aagcatcgactgctcACTGGATGGCGGCGTTAG (SEQ ID NO: 52) | |
| oTO187 | gagaacctaaaaattATCAAGCTTGCCTCGTCC (SEQ ID NO: 53) | |
| oTO188 | cgaggcaagcttgatAATTTTTAGGTTCTCTAGCC (SEQ ID NO: 54) | |
| oTO189 | gactctagaggatccCGGAGTGTCACATTTCTTTC (SEQ ID NO: 55) | |
| oTO190 | aaatgtgacactccgGGATCCTCTAGAGTCGAC (SEQ ID NO: 56) | |
| oTO191 | tactctgatgagtacGGTACCGAGCTCGAATTC (SEQ ID NO: 57) | |
| oTO410 | TAGCCTTGATTTGAGCAACCGGATCCTCTAGAGTCGACCT (SEQ ID NO: 58) | Assembly of pTO167 |
| oTO411 | TACAAATAGCAGTTATCAGCGGTACCGAGCTCGAATTCAC (SEQ ID NO: 59) | |
| oTO412 | GTGAATTCGAGCTCGGTACCGCTGATAACTGCTATTTGTATACA (SEQ ID NO: 60) | |
| oTO413 | tactaacgccgccatccagtTGATAAATGGAAAGTAGAGAAACAT (SEQ ID NO: 61) | |
| oTO414 | TCTCTACTTTCCATTTATCAactggatggcggcgttagta (SEQ ID NO: 62) | |
| oTO415 | AAATAATAATTGAGTAGGCCatcaagcttgcctcgtcc (SEQ ID NO: 63) | |
| oTO416 | ggacgaggcaagcttgatGGCCTACTCAATTATTATTTAATTTTTTT (SEQ ID NO: 64) | |
| oTO417 | AGGTCGACTCTAGAGGATCCGGTTGCTCAAATCAAGGCTA (SEQ ID NO: 65) | |
| oTO402 | GACTATCCTGAAAACGCTTGGGATCCTCTAGAGTCGACCT (SEQ ID NO: 66) | Assembly of pTO166 |
| oTO403 | GCGTTTACGGCGATTGCACCGGTACCGAGCTCGAATTCAC (SEQ ID NO: 67) | |
| oTO404 | GTGAATTCGAGCTCGGTACCGGTGCAATCGCCGTAAACGC (SEQ ID NO: 68) | |
| oTO405 | tactaacgccgccatccagtGAAAGATGATGGGAAACAAGGTGA (SEQ ID NO: 69) | |
| oTO406 | CTTGTTTCCCATCATCTTTCactggatggcggcgttagta (SEQ ID NO: 70) | |
| oTO407 | GGTATCATAAAAGCTCACGTatcaagcttgcctcgtcc (SEQ ID NO: 71) | |
| oTO408 | ggacgaggcaagcttgatACGTGAGCTTTTATGATACCT (SEQ ID NO: 72) | |
| oTO409 | AGGTCGACTCTAGAGGATCCCAAGCGTTTTCAGGATAGTC (SEQ ID NO: 73) | |
| oTO192 | ttcgagctcggtaccCCTTCTCGAGTTACTCTG (SEQ ID NO: 74) | Assembly of pTO145 |
| oTO193 | acgccgccatccagtTGGATAAAGCAAGTGAAAAAG (SEQ ID NO: 75) | |
| oTO194 | cacttgctttatccaACTGGATGGCGGCGTTAG (SEQ ID NO: 76) | |
| oTO195 | agtgggtatatggtcATCAAGCTTGCCTCGTCC (SEQ ID NO: 77) | |
| oTO196 | cgaggcaagcttgatGACCATATACCCACTCGC (SEQ ID NO: 78) | |
| oTO197 | gactctagaggatccCGCCACTTTACTTGCCAAC (SEQ ID NO: 79) | |
| oTO198 | gcaagtaaagtggcgGGATCCTCTAGAGTCGAC (SEQ ID NO: 80) | |
| oTO199 | agtaactcgagaaggGGTACCGAGCTCGAATTC (SEQ ID NO: 81) | |
| oTO216 | ttcgagctcggtaccCAGACATCTTTTGTGCAAG (SEQ ID NO: 82) | Assembly of pTO148 |
| oTO217 | acgccgccatccagtTTGTTGATGGGGAACTAC (SEQ ID NO: 83) | |
| oTO218 | gttccccatcaacaaACTGGATGGCGGCGTTAG (SEQ ID NO: 84) | |
| oTO219 | aaagtgacaccgtcaATCAAGCTTGCCTCGTCC (SEQ ID NO: 85) | |
| oTO220 | cgaggcaagcttgatTGACGGTGTCACTTTCGAG (SEQ ID NO: 86) | |
| oTO221 | gactctagaggatccGTGGTGTAAGCGATTGTG (SEQ ID NO: 87) | |
| oTO222 | aatcgcttacaccacGGATCCTCTAGAGTCGAC (SEQ ID NO: 88) | |
| oTO223 | cacaaaagatgtctgGGTACCGAGCTCGAATTC (SEQ ID NO: 89) | |
| oTO786 | acgacggccagtgaattcgagctcggtaccCGTATTCTCGAGCTCTTAAAG | Assembly of pTO202 |
| | (SEQ ID NO: 90) | |
| oTO787 | acgccgccatccagtAATGGTTGATTAACAAAAGAAAAAAAAAAAAAAG | |
| | (SEQ ID NO: 91) | |
| oTO788 | tgttaatcaaccattACTGGATGGCGGCGTTAG (SEQ ID NO: 92) | |
| oTO789 | gctaggaaaaattgcATCAAGCTTGCCTCGTCC (SEQ ID NO: 93) | |
| oTO790 | cgaggcaagcttgatGCAATTTTTCCTAGCCTTTATTTTTTC (SEQ ID NO: 94) | |
| oTO791 | gcatgcctgcaggtcgactctagaggatccCTACCAAAAGTTGACGTG (SEQ ID NO: 95) | |
| oTO678 | ggccagtgaattcgagctcggtaccGCAAACGTCAGTCTACCG (SEQ ID NO: 96) | Assembly of pTO188 |
| oTO679 | ttcgatactaacgccgccatccagtGATGGAATGGATGGAGAG (SEQ ID NO: 97) | |
| oTO680 | gagggtattctgggcctccatgtcgCTTTAAAATCGTTTTATTTAGTTCTG | |
| | (SEQ ID NO: 98) | |
| oTO681 | cctgcaggtcgactctagaggatccGTACAGCTTACACAACTAATAAAAAC | |
| | (SEQ ID NO: 99) | |
| oTO200 | ttcgagctcggtaccGGTCTTGTTAACATGGCC (SEQ ID NO: 100) | Assembly of pTO146 |
| oTO201 | acgccgccatccagtGATGCTCAATAGCTGAAG (SEQ ID NO: 101) | |
| oTO202 | cagctattgagcatcACTGGATGGCGGCGTTAG (SEQ ID NO: 102) | |
| oTO203 | aattggtacgttaggATCAAGCTTGCCTCGTCC (SEQ ID NO: 103) | |
| oTO204 | cgaggcaagcttgatCCTAACGTACCAATTCTC (SEQ ID NO: 104) | |
| oTO205 | gactctagaggatccCCAAAAATATGAAGACGAGAG (SEQ ID NO: 105) | |
| oTO206 | tcttcatatttttggGGATCCTCTAGAGTCGAC (SEQ ID NO: 106) | |
| oTO207 | catgttaacaagaccGGTACCGAGCTCGAATTC (SEQ ID NO: 107) | |
| oTO208 | ttcgagctcggtaccGCAGCTGCATTGGTACATATG (SEQ ID NO: 108) | Assembly of pTO147 |
| oTO209 | acgccgccatccagtGTGGAGCAGGTTGGGTTAG (SEQ ID NO: 109) | |
| oTO210 | cccaacctgctccacACTGGATGGCGGCGTTAG (SEQ ID NO: 110) | |
| oTO211 | tttgtgcatttaagtATCAAGCTTGCCTCGTCC (SEQ ID NO: 111) | |
| oTO212 | cgaggcaagcttgatACTTAAATGCACAAAACGC (SEQ ID NO: 112) | |
| oTO213 | gactctagaggatccGGACTACAGCTTCTTTGG (SEQ ID NO: 113) | |
| oTO214 | aagaagctgtagtccGGATCCTCTAGAGTCGAC (SEQ ID NO: 114) | |
| oTO215 | taccaatgcagctgcGGTACCGAGCTCGAATTC (SEQ ID NO: 115) | |
| oTO819 | acgttgtaaaacgacggccagtgaattcgagctcggtaccCAAAGTAGCCACACACTTG | Assembly of pTO205 |
| | (SEQ ID NO: 116) | |
| oTO820 | tactaacgccgccatccagtATTTGGCAGAGAGTAGAATG (SEQ ID NO: 117) | |
| oTO821 | cattctactctctgccaaatACTGGATGGCGGCGTTAG (SEQ ID NO: 118) | |
| oTO822 | tgtgcagccttgtatcaacaATCAAGCTTGCCTCGTCC (SEQ ID NO: 119) | |
| oTO823 | ggggacgaggcaagcttgatTGTTGATACAAGGCTGCAC (SEQ ID NO: 120) | |
| oTO824 | cgccaagcttgcatgcctgcaggtcgactctagaggatccCTTCGTCCTCGTGCTTGG | |
| | (SEQ ID NO: 121) | |
| oTO825 | acgttgtaaaacgacggccagtgaattcgagctcggtaccGAACGCAACAACTTAAGC | Assembly of pTO206 |
| | (SEQ ID NO: 122) | |
| oTO826 | tactaacgccgccatccagtGCGTTTGTGTTTGATGGAG (SEQ ID NO: 123) | |
| oTO827 | tctccatcaaacacaaacgcACTGGATGGCGGCGTTAG (SEQ ID NO: 124) | |
| oTO828 | ataaaggtttcgtcttttgtATCAAGCTTGCCTCGTCC (SEQ ID NO: 125) | |
| oTO829 | ggggacgaggcaagcttgatACAAAAGACGAAACCTTTATATAAC (SEQ ID NO: 126) | |
| oTO830 | aagcttgcatgcctgcaggtcgactctagaggatccCCTAAGATATTCAAGAGGAAATC | |
| | (SEQ ID NO: 127) | |
| oTO831 | gttgtaaaacgacggccagtgaattcgagctcggtaccGGTACCAGAATGTGGGCTTTC | Assembly of pTO207 |
| | (SEQ ID NO: 128) | |
| oTO832 | tactaacgccgccatccagtCGTGAGGCGAGGCAGATC (SEQ ID NO: 129) | |
| oTO833 | aagatctgcctcgcctcacgACTGGATGGCGGCGTTAG (SEQ ID NO: 130) | |
| oTO834 | caaagggaccgtggctgcatATCAAGCTTGCCTCGTCC (SEQ ID NO: 131) | |
| oTO835 | ggggacgaggcaagcttgatATGCAGCCACGGTCCCTT (SEQ ID NO: 132) | |
| oTO836 | gcttgcatgcctgcaggtcgactctagaggatccGGTTTACGTTGCCACACTATTCTG | |
| | (SEQ ID NO: 133) | |
| oTO879 | acgttgtaaaacgacggccagtgaattcgagctcggtaccCAGTGGAGCGGAAACTCTTC | Assembly of pTO211 |
| | (SEQ ID NO: 134) | |
| oTO880 | actgctgtcgattcgatactaacgccgccatccagtTGTGGAGGTGAAGTTTTAAGATAG | |
| | (SEQ ID NO: 135) | |
| oTO881 | ctggccgggtgacccggcggggacgaggcaagcttgatTTCTAATGACTGATACTCATAC | |
| | (SEQ ID NO: 136) | |
| oTO882 | cgccaagcttgcatgcctgcaggtcgactctagaggatccGTGGACCTGATTTGACTG | |
| | (SEQ ID NO: 137) | |
| oTO937 | gtgaattcgagctcggtaccCCTGAATCTCTCAACGAAG (SEQ ID NO: 138) | Assembly of pTO222 |
| oTO938 | tactaacgccgccatccagtTCAGAACATGGCAAATAATG (SEQ ID NO: 139) | |
| oTO943 | accacaatcgcttacaccacGGATCCTCTAGAGTCGAC (SEQ ID NO: 140) | |
| oTO944 | tcttcgttgagagattcaggGGTACCGAGCTCGAATTC (SEQ ID NO: 141) | |
| oTO927 | tatactgctgtcgattcgatactaacgccgccatccagtTGACGGTGTCACTTTCGAG | Assembly of pTO221 |
| | (SEQ ID NO: 142) | |
| oTO928 | gtcaagactgtcaaggagggtattctgggcctccatgtcgTTGTTGATGGGGAACTAC | |
| | (SEQ ID NO: 143) | |
| oTO875 | acgttgtaaaacgacggccagtgaattcgagctcggtaccGTCGGGTTCCTGAGAAATG | Assembly of pTO210 |
| | (SEQ ID NO: 144) | |
| oTO876 | ctatactgctgtcgattcgatactaacgccgccatccagtGGGAGGAATTTGGGTGGATG | |
| | (SEQ ID NO: 145) | |
| oTO877 | cgctggccgggtgacccggcggggacgaggcaagcttgatACACCGTGAGGTGACACC | |
| | (SEQ ID NO: 146) | |
| oTO878 | caagcttgcatgcctgcaggtcgactctagaggatccGCATACTTTCTTCACAGTTGGTG | |
| | (SEQ ID NO: 147) | |
| oTO972 | ctggccgggtgacccggcggggacgaggcaagcttgatGTCATGTAAATATTCTTAGAGC | Assembly of pTO225 |
| | (SEQ ID NO: 148) | |
| oTO973 | cgccaagcttgcatgcctgcaggtcgactctagaggatccCCGTATTCTAGGGATGTC | |
| | (SEQ ID NO: 149) | |
| oTO974 | acgttgtaaaacgacggccagtgaattcgagctcggtaccCACCGACTTTTTCAAGGC | |
| | (SEQ ID NO: 150) | |
| oTO975 | ctatactgctgtcgattcgatactaacgccgccatccagtCCAGGAGTGGTAACGGTG | |
| | (SEQ ID NO: 151) | |
| oTO857 | GACGTTGTAAAACGACGGCCAGTGAATTCGAGCTCGGTACCCCTGTCAAGATAT | Assembly of pTO208 |
| | TTGGC (SEQ ID NO: 152) | |
| oTO858 | cggcggggacgaggcaagcttgatGTTTTGCTTTATATTGAACTC | |
| | (SEQ ID NO: 153) | |
| oTO859 | GAGTTCAATATAAAGCAAAACatcaagcttgcctcgtccccgccg (SEQ ID NO: 154) | |
| oTO860 | CACAACGAAAGAATTGCTGTCCactggatggcggcgttagtatcg (SEQ ID NO: 155) | |
| oTO861 | cgatactaacgccgccatccagtGGACAGCAATTCTTTCGTTGTG (SEQ ID NO: 156) | |
| oTO862 | CAAGCTTGCATGCCTGCAGGTCGACTCTAGAGGATCCGTTTCGATGCCTCAGGG | |
| | TAATC (SEQ ID NO: 157) | |
| oTO1317 | cgacggccagtgaattcgagctcggtaccGCCACAGCGTGACGTGAATTTC | Assembly of pTO270 |
| | (SEQ ID NO: 158) | |
| oTO1318 | gctgtcgattcgatactaacgccgccatccagtGGGATGCTGGGTAGGACG | |
| | (SEQ ID NO: 159) | |
| oTO1319 | gggtgacccggcggggacgaggcaagcttgatGAGGTTTTGGGACGTTGG | |
| | (SEQ ID NO: 160) | |
| oTO1320 | gcttgcatgcctgcaggtcgactctagaggatccTGTGTGGGATTAGCTTTG | |
| | (SEQ ID NO: 161) | |
| oTO978 | GCTTTTACGACCTTATATGC (SEQ ID NO: 162) | Assembly of pTO224 |
| oTO979 | ACAAACTGTGGAATTGACGC (SEQ ID NO: 163) | |
| oTO980 | gTTACCCATAGCATTAATGTGgttttagagctagaaatagcaag (SEQ ID NO: 164) | |
| oTO981 | CACATTAATGCTATGGGTAAcaaattaaaaatagtttacgcaagtc (SEQ ID NO: 165) | |
| oTO6 | Gactgtcaaggagggtattc (SEQ ID NO: 166) | Generation of |
| oTO7 | CCGCaagtgattagacttag (SEQ ID NO: 167) | CaTO227 |
| oTO8 | Gaataccacttgtttaccgg (SEQ ID NO: 168) | |
| oTO9 | GGTGGCGGCAAAACTAATTC (SEQ ID NO: 169) | |
| oTO40 | CGCTATATCCTTCTTGGTCG (SEQ ID NO: 170) | |
| oTO41 | Gtcccaaaaccttctcaagc (SEQ ID NO: 171) | |
| oTO652 | CATCAGAATTGTTCAAACAACTACCAATTGTTAATATCAGggaaacagctatgaccatg | |
| | (SEQ ID NO: 172) | |
| oTO653 | GAACTAGATCAAGCCAAAAAGGTGATCATAACAATATAGTCAgtaaaacgacggcca | |
| | g (SEQ ID NO: 173) | |
| oTO698 | CCTGGTGGTGCAGTTACAGTcaaattaaaaatagtttacgcaagtc (SEQ ID NO: 174) | |
| oTO699 | gACTGTAACTGCACCACCAGGgttttagagctagaaatagcaag (SEQ ID NO: 175) | |
| oTO1480 | Acgttgtaaaacgacggccagtgaattcgagctcggtacccagataactcaccacgag | Assembly of pTO288 |
| | (SEQ ID NO: 176) | |
| oTO1481 | Ggggacgaggcaagcttgatgaaagatgatgggaaacaag (SEQ ID NO: 177) | |
| oTO1482 | Cttgtttcccatcatctttcatcaagcttgcctcgtcc (SEQ ID NO: 178) | |
| oTO1483 | Gcaagcgaagcaagtttcatttttgttagtttttgaggtgatacag (SEQ ID NO: 179) | |
| oTO1484 | Cacctcaaaaactaacaaaaatgaaacttgcttcgcttg (SEQ ID NO: 180) | |
| oTO1485 | Cgccaagcttgcatgcctgcaggtcgactctagaggatccgtctacagtggttgagaac | |
| | (SEQ ID NO: 181) | |
| oTO1274 | ccggtcttcttttgctctaaGTAGCAGCATGATTATGAAC (SEQ ID NO: 182) | Assembly of pTO264 |
| oTO1275 | agaagaagactgtatttcatTGTGGAGGTGAAGTTTTAAG (SEQ ID NO: 183) | |
| oTO1276 | cttaaaacttcacctccacaATGAAATACAGTCTTCTTCTTC (SEQ ID NO: 184) | |
| oTO1277 | gttcataatcatgctgctacTTAGAGCAAAAGAAGACC (SEQ ID NO: 185) | |
| oTO1150 | ggccgggtgacccggcggggacgaggcaagcttgatTGTGGAGGTGAAGTTTTAAGATAG | Assembly of pTO250 |
| | (SEQ ID NO: 186) | |
| oTO1151 | ctatactgctgtcgattcgatactaacgccgccatccagtCACATGGGCTCCGTTTCTG | |
| | (SEQ ID NO: 187) | |
| oTO1152 | agaagtaaagagaatctcatTTTTGTTAGTTTTTGAGGTGATACAGAAAAAG | |
| | (SEQ ID NO: 188) | |
| oTO1153 | cacctcaaaaactaacaaaaATGAGATTCTCTTTACTTCTTGC (SEQ ID NO: 189) | |
| oTO1154 | cgccaagcttgcatgcctgcaggtcgactctagaggatccGCAAGACGAACCTCCAAC | |
| | (SEQ ID NO: 190) | |
| oTO1078 | tcgttaactgtgtattacctgttcataatcatgctgctacTTAAAGCAACAAGGCAGC | Assembly of pTO223 |
| | (SEQ ID NO: 191) | |
| oTO1066 | GTAGCAGCATGATTATGAACAGG (SEQ ID NO: 192) | |
| oTO1077 | tgctgtcgattcgatactaacgccgccatccagtTTCTAATGACTGATACTCATACTTTC | |
| | (SEQ ID NO: 193) | |
| oTO1430 | TTAAAGCAACAAGGCAGC (SEQ ID NO: 194) | Assembly of pTO284 |
| oTO1431 | TACACCTGCAAGAAGTAAAGAGAATCTCATTGTGGAGGTGAAGTTTTAAG | |
| | (SEQ ID NO: 195) | |
| oTO1432 | CTTTGCTTGCTGCTGCCTTGTTGCTTTAAGTAGCAGCATGATTATGAAC | |
| | (SEQ ID NO: 196) | |
| oTO1159 | gactacaaggacgacgatgacaagACAACCACCACTACTTCG (SEQ ID NO: 197) | Assembly of pTO280 |
| oTO1160 | cttgtcatcgtcgtccttgtagtcTTCACACTCACACACCCAAC (SEQ ID NO: 198) | |
| oTO1427 | CAGTTAATAGTTTGCGCAACG (SEQ ID NO: 199) | Assembly of pTO281 |
| oTO1428 | CTGTAGCAATGGCAACAAC (SEQ ID NO: 200) | |
| oTO1417 | CCGGCGTTGTGTGCTGTCTCCCACATGCTTTGGAGGTTCGTCTTGCTTTGGAGCC | |
| | AG (SEQ ID NO: 201) | |
| oTO1418 | GAACCTCCAAAGCATGTGGGAGACAGCACACAACGCCGGCTGGTTTGTGCTTG | |
| | TGTCTC (SEQ ID NO: 202) | |
| oTO1421 | AACGGATGCTCGAGGCTGCTCACGCTCACGCTCCAGCCGGCGTTGTGTGCTGTC | Assembly of pTO282 |
| | TCCCAC (SEQ ID NO: 203) | |
| oTO1422 | CCGGCTGGAGCGTGAGCGTGAGCAGCCTCGAGCATCCGTTCCGACCTCACTCTC | |
| | ACTCTG (SEQ ID NO: 204) | |
| oTO1425 | GCCATCATGCTGCTTCCAACGATGGCCCCGAGTTTATCGATGCGGGAG | Assembly of pTO283 |
| | (SEQ ID NO: 205) | |
| oTO1426 | GGGGCCATCGTTGGAAGCAGCATGATGGCCACCTTTCACCCAGTAGTG | |
| | (SEQ ID NO: 206) | |

Cassettes for transformation of C. auris were maintained in the multiple cloning site of the pUC19 cloning vector and assembled from fragments using the NeBuilder HIFI DNA Assembly Master Mix (NEB #E2621) or the Codex DNA Gibson Assembly Ultra Master Mix (Codex DNA #GA1200) according to the manufacturer's instructions.

### Plasmid and Strain Construction

pTO144/CauTO186: The plasmid backbone was amplified from pTO139 using oTO190-oTO191. The NAT cassette was amplified from pTO137 using oTO186-oTO187. The NAT cassette was flanked by approximately 500 bp regions homologous to the regions immediately upstream and downstream of ALS (B9J08_002582) amplified from C. auris genomic DNA using oTO184-oTO185 and oTO188-oTO189 respectively. The repair cassette was amplified using oTO18-oTO19 and transformed into AR0382 to generate CauTO186.

pTO167/CauTO187: The plasmid backbone was amplified from pTO139 using oTO410-oTO41 1. The NAT cassette was amplified from pTO137 using oTO414-oTO415. The NAT cassette was flanked by approximately 500 bp regions homologous to the regions immediately upstream and downstream of ALS (B9J08_004498) amplified from C. auris genomic DNA using oTO412-oTO413 and oTO416-oTO417 respectively. The repair cassette was amplified using oTO18-oTO19 and transformed into AR0382 to generate CauTO187.

pTO166/CauTO226: The plasmid backbone was amplified from pTO139 using oTO402-oTO403. The NAT cassette was amplified from pTO137 using oTO406-oTO407. The NAT cassette was flanked by approximately 500 bp regions homologous to the regions immediately upstream and downstream of ALS (B9J08_004112) amplified from C. auris genomic DNA using oTO404-oTO404 and oTO408-oTO409 respectively. The repair cassette was amplified using oTO18-oTO19 and transformed into AR0382 to generate CauTO226.

pTO145/CauTO233: The plasmid backbone was amplified from pTO139 using oTO198-oTO199. The NAT cassette was amplified from pTO137 using oTO194-oTO195. The NAT cassette was flanked by approximately 500 bp regions homologous to the regions immediately upstream and downstream of IFF (B9J08_004100) amplified from C. auris genomic DNA using oTO192-oTO193 and oTO196-oTO197 respectively. The repair cassette was amplified using oTO18-oTO19 and transformed into AR0382 to generate CauTO233.

pTO148/CauTO234: The plasmid backbone was amplified from pTO139 using oTO222-oTO223. The NAT cassette was amplified from pTO137 using oTO218-oTO219. The NAT cassette was flanked by approximately 500 bp regions homologous to the regions immediately upstream and downstream of IFF (B9J08_004109) amplified from C. auris genomic DNA using oTO216-oTO217 and oTO220-oTO221 respectively. The repair cassette was amplified using oTO18-oTO19 and transformed into AR0382 to generate CauTO234.

pTO202/CauTO235: The plasmid backbone was amplified from pTO139 using oTO590-oTO591. The NAT cassette was amplified from pTO137 using oTO788-oTO789. The NAT cassette was flanked by approximately 500 bp regions homologous to the regions immediately upstream and downstream of IFF (B9J08_004098) amplified from C. auris genomic DNA using oTO786-oTO787 and oTO790-oTO791 respectively. The repair cassette was amplified using oTO18-oTO19 and transformed into AR0382 to generate CauTO235.

pTO188/CauTO236: The plasmid backbone was amplified from pTO139 using oTO590-oTO591. The NEO cassette was amplified from pTO169 using oTO668-oTO669. The NEO cassette was flanked by approximately 500 bp regions homologous to the regions immediately upstream and downstream of IFF (B9J08_004110) amplified from C. auris genomic DNA using oTO678-oTO679 and oTO680-oTO681 respectively. The repair cassette was amplified using oTO18-oTO19 and transformed into AR0382 to generate CauTO236.

pTO146/CauTO247: The plasmid backbone was amplified from pTO139 using oTO206-oTO207. The NAT cassette was amplified from pTO137 using oTO202-oTO203. The NAT cassette was flanked by approximately 500 bp regions homologous to the regions immediately upstream and downstream of IFF (B9J08_001531) amplified from C. auris genomic DNA using oTO200-oTO201 and oTO204-oTO205 respectively. The repair cassette was amplified using oTO18-oTO19 and transformed into AR0382 to generate CauTO247.

pTO147/CauTO248: The plasmid backbone was amplified from pTO139 using oTO214-oTO215. The NAT cassette was amplified from pTO137 using oTO210-oTO211. The NAT cassette was flanked by approximately 500 bp regions homologous to the regions immediately upstream and downstream of IFF (B9J08_004892) amplified from C. auris genomic DNA using oTO208-oTO209 and oTO212-oTO213 respectively. The repair cassette was amplified using oTO18-oTO19 and transformed into AR0382 to generate CauTO248.

pTO205/CauTO249: The plasmid backbone was amplified from pTO139 using oTO590-oTO591. The NAT cassette was amplified from pTO137 using oTO821-oTO822. The NAT cassette was flanked by approximately 500 bp regions homologous to the regions immediately upstream and downstream of IFF (B9J08_001155) amplified from C. auris genomic DNA using oTO819-oTO820 and oTO823-oTO824 respectively. The repair cassette was amplified using oTO18-oTO19 and transformed into AR0382 to generate CauTO249.

pTO206/CauTO250: The plasmid backbone was amplified from pTO139 using oTO590-oTO591. The NAT cassette was amplified from pTO137 using oTO827-oTO828. The NAT cassette was flanked by approximately 500 bp regions homologous to the regions immediately upstream and downstream of IFF (B9J08_004451) amplified from C. auris genomic DNA using oTO825-TO826 and oTO829-oTO830 respectively. The repair cassette was amplified using oTO18-oTO19 and transformed into AR0382 to generate CauTO250.

pTO207/CauTO251: The plasmid backbone was amplified from pTO139 using oTO590-oTO591. The NAT cassette was amplified from pTO137 using oTO833-oTO834. The NAT cassette was flanked by approximately 500 bp regions homologous to the regions immediately upstream and downstream of IFF (B9J08_000675) amplified from C. auris genomic DNA using oTO831-TO832 and oTO835-oTO836 respectively. The repair cassette was amplified using oTO18-oTO19 and transformed into AR0382 to generate CauTO250.

pTO211/CauTO261: The plasmid backbone was amplified from pTO139 using oTO590-oTO591. The NAT cassette was amplified from pTO137 using oTO668-oTO874. The NAT cassette was flanked by approximately 500 bp regions homologous to the regions immediately upstream and downstream of SCF1 (B9J08_001458) amplified from C. auris genomic DNA using oTO879-TO880 and oTO881-oTO882 respectively. The repair cassette was amplified using oTO18-oTO19 and transformed into AR0382 to generate CauTO261.

CauTO320: A FLAG-tagged SCF1 complementation cassette was generated using overlap extension PCR to insert a FLAG sequence between domains A and B (between amino acids 250-251). Fragments were amplified from pTO223 using oTO1160-oTO224 and oTO1159-oTO225 and fused with extension primers oTO945-oTO946. The resulting fusion fragment was transformed into CauTO261 to generate CauTO320.

pTO222/CauTO307: The ORF for IFF (B9J08_004109) along with approximately 500 bp of upstream sequence was amplified from C. auris genomic DNA using oTO937-oTO938. The NEO cassette was amplified from pTO169 using oTO668-oTO669. The plasmid backbone was amplified from pTO139 using oTO943-oTO944. The repair cassette was amplified using oTO947-oTO948 and transformed into CauTO234 to generate CauTO307.

pTO221/CauTO270: The vector including approximately 500 bp regions homologous to the regions immediately upstream and downstream of IFF (B9J08_004109) was amplified from pTO148 using oT0927-oTO928. The NEO cassette was amplified from pTO169 using oT0668-oTO669. The repair cassette was amplified using oTO18-oTO19 and transformed into CauTO261 to generate CauTO270.

pTO210/CauTO260: The plasmid backbone was amplified from pTO139 using oTO590-oTO591. The NAT cassette was amplified from pTO137 using oT0668-oTO874. The NAT cassette was flanked by approximately 500 bp regions homologous to the regions immediately upstream and downstream of CRZ2 (B9J08_000447) amplified from C. auris genomic DNA using oTO875-oTO876 and oTO877-oTO878 respectively. The repair cassette was amplified using oTO18-oTO19 and transformed into AR0382 to generate CauTO186.

pTO225/CauTO272: The plasmid backbone was amplified from pTO139 using oT0590-oTO591. The NAT cassette was amplified from pTO137 using oTO668-oTO874. The NAT cassette was flanked by approximately 500 bp regions homologous to the regions immediately upstream and downstream of UME6 (B9J08_000592) amplified from C. auris genomic DNA using oT0974-oTO975 and oTO972-oTO973 respectively. The repair cassette was amplified using oTO18-oTO19 and transformed into AR0382 to generate CauTO272.

pTO208/CauTO278: The plasmid backbone was amplified from pTO139 using oT0590-oT0591. The NAT cassette was amplified from pTO137 using oTO859-oTO860. The NAT cassette was flanked by approximately 500 bp regions homologous to the regions immediately upstream and downstream of RIM101 (B9J08_003060) amplified from C. auris genomic DNA using oTO857-oTO858 and oTO861-oTO862 respectively. The repair cassette was amplified using oTO18-oTO19 and transformed into AR0382 to generate CauTO278.

pTO270/CauTO408: The plasmid backbone was amplified from pTO139 using oTO590-oTO591. The NAT cassette was amplified from pTO137 using oT0668-oTO874. The NAT cassette was flanked by approximately 500 bp regions homologous to the regions immediately upstream and downstream of CZF1 (B9J08_003772) amplified from C. auris genomic DNA using oTO1317-oTO1318 and oTO1319-oTO1320 respectively. The repair cassette was amplified using oTO18-oTO19 and transformed into AR0382 to generate CauTO408.

pTO224/CauTO288: The plasmid backbone was amplified from pTO139 using oTO590-oTO591. The NAT cassette was amplified from pTO137 using oTO668-oTO874. The NAT cassette was flanked by approximately 500 bp regions homologous to the regions immediately upstream and downstream of IRF1 (B9J08_004787) amplified from C. auris genomic DNA using oTO968-oTO969 and oTO970-oTO971 respectively. The repair cassette was amplified using oTO18-oTO19 and transformed into AR0382 to generate CauTO288.

CaTO227: The ALS1 deletion cassette with NAT was amplified from pTO100 using oTO652-oTO653. The sgRNA guide was amplified from pTO102 using oTO6-oTO698 and oTO8-oTO699, and the fusion was amplified using oTO7-oTO9. Cas9 was amplified from pTO102 using oTO40-oTO41. The three linear pieces of DNA were transformed into SC5314 using a PEG-heat shock transformation, as described previously (68).

pTO288/CauTO436: The plasmid backbone was amplified from pTO139 using oTO590-oTO591. The NAT cassette and pCauTEF1 promoter were amplified from pTO250 using oTO1482-oTO1483. Flanking regions of approximately 500 bp were amplified from C. auris genomic DNA using oTO1480-oTO1481 and oTO1484-oTO1485. The repair cassette was amplified using oTO18-19 and transformed into AR0382 to generate CauTO436.

pTO255/ChTO346: The plasmid backbone was amplified from pTO139 using oTO590-oTO591. The NAT cassette was amplified from pTO137 using oTO668-oTO874. The NAT cassette was flanked by approximately 500 bp regions homologous to the regions immediately upstream and downstream of SCF1 (CXQ85_003100) amplified from C. haemulonii genomic DNA using oTO1223-oTO1224 and oTO1225-oTO1226 respectively. The repair cassette was amplified using oTO18-oTO19 and transformed into AR0395 to generate ChTO346.

pTO264/CauTO364: The plasmid backbone was amplified from pTO139 using oTO590-oTO591. C. haemulonii SCF1 was amplified from AR0395 gDNA using oTO1276-oTO1277. The ADH1 terminator, NEO cassette, and approximately 500 bp of SCF1 downstream intragenic region were amplified from pTO223 using oTO1274-oTO882. Approximately 500 bp SCF1 upstream flanking region was amplified from C. auris gDNA using oTO879-oTO1275. The repair cassette was amplified using oTO945-946 and transformed into CauTO261 to generate CauTO364.

pTO250/CauTO308, CauTO312, CauTO323: The plasmid backbone was amplified from pTO139 using oTO590-oTO591. Approximately 500 bp homologous to the region immediately upstream of SCF1 (B9J08_001458) was amplified from C. auris genomic DNA using oTO879-oTO1150. The NAT cassette was amplified from pTO137 using oTO668-oTO875. 1000 bp of promoter sequence upstream of CauTEF1 (B9J08_003610) was amplified from C. auris genomic DNA using oTO1151-1152. The first 506 bp of the SCF1 ORF was amplified using oTO1153-oTO1154. The repair cassette was amplified using oTO945-oTO1161 and transformed into AR0387 to generate CauTO308 and into AR0381 to generate CauTO312. The entire promoter replacement cassette and SCF1 ORF was amplified from CauTO308 genomic DNA using oTO945-oTO946 and transformed into AR0381 to generate CauTO323.

pTO223/CauTO306: The ORF for SCF1 (B9J08_001458) along with approximately 500 bp of upstream sequence was amplified from C. auris genomic DNA using oTO879-oTO1078. The ADH1 terminator and NEO cassette were amplified from pTO169 using oTO1066-oTO668. Approximately 500 bp homologous to the region immediately downstream of SCF1 was amplified from C. auris genomic DNA using oTO1077-oTO882. The plasmid backbone was amplified from pTO139 using oTO590-oTO591. The repair cassette was amplified using oTO945-oTO946 and transformed into CauTO261 to generate CauTO306.

pTO284/CauTO438: SCF1 was amplified from AR0381 gDNA using oTO1212-oTO1430 and assembled into the vector amplified from pTO223 using oTO1431-oTO1432. The repair cassette was amplified using oTO945-oTO946 and transformed into CauTO261 to generate CauTO438.

pTO280: The SCF1 complementation vector was amplified from pTO223 using oTO1159-oTO1160, which incorporate a 1× FLAG tag at the A-B Domain junction. The resultant product was assembled in a single fragment Gibson assembly and used as template for site-directed mutagenesis.

pTO281/CauTO433: The entire pTO280 vector was amplified in two overlapping fragments using overlapping backbone-specific primers oTO1427 and oTO1428 paired with oTO1418 and oTO1417, respectively. oTO1418 and oTO1417 contain overlapping sequence with the point mutations instantiated. The repair cassette was amplified using oTO945-oTO946 and transformed into CauTO261 to generate CauTO433.

pTO282/CauTO434: The entire pTO280 vector was amplified in two overlapping fragments using overlapping backbone-specific primers oTO1427 and oTO1428 paired with oTO1422 and oTO1421, respectively. oTO1422 and oTO1421 contain overlapping sequence with the point mutations instantiated. The repair cassette was amplified using oTO945-oTO946 and transformed into CauTO261 to generate CauTO434.

pTO283/CauTO430: The entire pTO280 vector was amplified in two overlapping fragments using overlapping backbone-specific primers oTO1427 and oTO1428 paired with oTO1426 and oTO1425, respectively. oTO1426 and oTO1425 contain overlapping sequence with the point mutations instantiated. The repair cassette was amplified using oTO945-oTO946 and transformed into CauTO261 to generate CauTO430.

pTO268/CauTO432: The N-terminal domain of SCF1 was synthesized with the point mutations instantiated and cloned into the PCR product of pTO223 generated from oTO1159-591. The repair cassette was amplified using oTO945-oTO946 and transformed into CauTO261 to generate CauTO430.

C. auris Transformation

C. auris transformation was performed using a transient-Cas9 expression approach as described previously (38). Briefly, transformation repair cassettes were amplified from assembled plasmids. A Cas9 expression cassette was amplified from pTO135 using oTO143-oTO41. Cassettes for the expression of sgRNA targeting specific loci were amplified from pTO136 using overlap-extension PCR to change the gRNA sequence. All linear PCR products were purified using a Zymo DNA Clean & Concentrator kit (Cat no. D4034, Zymo Research) according to the manufacturer's instructions.

To prepare electro-competent cells, C. auris cells were incubated in liquid YPD at 30° C. overnight with gentle agitation. Cells were harvested by centrifugation and resuspended in TE buffer with 100 mM Lithium Acetate and incubated at 30° C. for 1 hr with constant shaking. DTT was added to the cells at a final concentration of 25 mM before further incubating at 30° C. for 30 min. Cells were harvested by centrifugation at 4° C. before being washed once with ice-cold water and once with ice-cold 1 M Sorbitol. Harvested cells were resuspended in ice-cold 1M sorbitol and maintained on ice for immediate usage or aliquoted and stored at −80° C. for up to several months before transformation.

Electroporation was performed by adding 45 μL competent cells to a pre-chilled 2 mm-gap electro-cuvette along with 500-1000 ng each of the PCR-amplified Cas9, sgRNA, and repair cassettes. Cells were electroporated using a Bio-Rad MicroPulser Electroporator according to the pre-defined P. pastoris (PIC) protocol (2.0 kV, 1 pulse). Electroporated cells were recovered in 1 M Sorbitol then resuspended in YPD and allowed 2 hrs of outgrowth at 30° C. with constant rotation. Outgrown cells were spread-plated on selective media. For repair cassettes encoding the NAT marker, cells were selected on YPD+200 μg/mL nourseothricin and incubated at 30° C. for 2-3 days. For repair cassettes encoding the NEO marker, cells were selected on YPD+1 mg/mL G418 and incubated at 23° C. for 4 days or YPD+1 mg/mL G418+1 mg/mL Molybdate (69) and incubated at 30° C. for 2-3 days. Transformant colonies were passaged to isolation and correct incorporation of the repair cassette was confirmed for each by colony PCR using Phire Plant Direct PCR Master Mix (F160; Thermo Fisher Scientific) according to the manufacturer's instructions. Each mutant was confirmed by at least three independent PCR reactions with distinct primer sets specific to the mutation site and compared to parental strains. Deletion mutants were confirmed with gene-specific primers based on absence of amplification from the ORF and integration of the repair cassette at the genetic locus. For mutants encoding site-directed mutations, genomic DNA was isolated and the entire genetic locus spanning the gene and sequence surrounding the integration junctions was PCR-amplified and sequenced using long-read sequencing to confirm the site-directed mutations were intact and no unintended mutations were present at the locus.

C. haemulonii Transformation

C. haemulonii transformation was performed similarly to C. auris transformation, except the outgrowth was extended to 5 hrs and transformants were selected on YPD+50 μg/mL nourseothricin at 30° C. for 3 days.

Agrobacterium tumefaciens-Mediated Transformation (AtMT)

AtMT was performed as described previously (38). Briefly, A. tumefaciens strain pTO131 (EHA 105 harboring pTO128) was grown overnight at 30° C. in liquid LB media containing kanamycin. A. tumefaciens cells were harvested by centrifugation, washed once with sterile water, then resuspended at a final OD₆₀₀ of 0.15 in liquid Induction Medium (IM) supplemented with 100 M acetosyringone 3′,5′-dimethoxy-4-hydroxyacetophenone (AS) and incubated at room temperature for 6 hrs with constant agitation. Recipient C. auris AR0382 cells were grown overnight at 30° C. in YPD and harvested by centrifugation then resuspended in sterile water at a final OD₆₀₀ of 1.0. Prepared A. tumefaciens and C. auris cells were combined at equal volumes and the mixed culture was incubated on solid IM Agar supplemented with AS at 23° C. for 4 days. Cells were harvested into liquid YPD. The resulting suspension was washed three times by low-speed centrifugation to separate fungal cells from bacterial cells and aliquots of the washed culture were spread-plated on YPD+200 μg/mL nourseothricin+200 μg/mL cefotaxime. Plates were incubated at 30° C. for 2 days. Transformant colonies were manually arrayed into 96 well plates and grown overnight in YPD at 30° C. Each well was overlayed with 50% glycerol and arrayed plates were frozen and stored at −80° C.

### AtMT Transgene Insertion Site Identification

Identification of transgene insertion sites was performed as described previously (38). Briefly, genomic DNA was isolated from mutants of interest and sequenced by Illumina sequencing. Library preparation, quality control, and Whole Genome Sequencing were performed by SeqCenter (Pittsburgh, PA, USA). Library preparation was performed based on the Illumina Nextera kit and sequencing performed on the Nextseq 550 platform to generate 150 bp paired-end sequencing reads. Sequencing data was analyzed using the Galaxy web platform public server at usegalaxy.org (70). Read quality was assessed using FastQC and reads were trimmed using Trimmomatic (71) with a Phred quality cutoff of 20. Processed reads were then mapped to a linearized reference sequence of pTO128 (pPZP-Nat) using the Burrows-Wheeler Aligner with maximum exact matches (BWA-MEM) (72) configured with minimum seed length=50 and band width=2. Soft clipped read sequence corresponding to genomic DNA neighboring the T-DNA integration junction was extracted from the aligned BAM file using the extractSoftClipped script from SE-MEI. The resulting sequences were mapped back to the C. auris B8441 reference assembly (NCBI GCA_002759435.2) using BWA-MEM with the default configuration to identify integration sites. T-DNA integration loci were confirmed for each sequenced mutant with Sanger sequencing.

### RNA Extraction

RNA extraction was performed using a formamide extraction method (73). Briefly, cultured cells were harvested by centrifugation and all media was removed. Dry cell pellets were frozen on dry ice and stored at −80° C. before processing. To extract RNA, cell pellets were thawed at room temperature and resuspended in 100 μL FE Buffer (98% formamide, 0.01M EDTA). 50 μL of 500 μm RNAse-free glass beads was added to this suspension and the mixture was homogenized for 30 sec 3 times using a BioSpec Mini-Beadbeater-16 (Biospec Products Inc., Bartlesville, OK, USA). The resulting cell lysate was clarified by centrifugation to remove cell debris. The supernatant was collected as the crude RNA extract. The crude extract was purified using a Qiagen RNeasy mini kit (ref 74104, Qiagen) according to the manufacturer's instructions. Samples were DNAse treated with Invitrogen DNAse (RNAse free) (Qiagen, cat no. 79254). The integrity and purity of the extracted RNA was confirmed via Nanodrop and agarose gel electrophoresis prior to downstream applications.

### RT-qPCR

Purified RNA from cells cultured overnight in YPD at 30° C. was used to generate cDNA using the iScript cDNA synthesis kit (cat. 1708890, Bio-Rad Laboratories, Inc., Hercules, CA, USA) according to the manufacturer's instructions. Primers specific to target genes were designed using NCBI primer blast (74) with the C. auris B8441 assembly (NCBI GCA_002759435.2) or the C. haemulonii B11899 assembly (NCBI GCA_002926055.1) as a reference. Prepared cDNA was used as a template in qPCR reactions with the Power-Up SYBR Green Master Mix (cat. A25741, ThermoFisher Scientific, Waltham, MA, USA) according to the manufacturer's specifications. Cycling was performed using a Bio-Rad CFX Opus 384 Real Time PCR System. For C. auris, amplification of ACT1 was measured using primers oTO359-oTO360, of SCF1 using primers oTO1251-oTO1252, and of IFF4109 using primers oTO615-oTO616. For C. haemulonii, amplification of ACT1 was measured using primers oTO1253-oTO1254 and amplification of SCF1 was measured using primers oTO1229-oTO1230.

### RNA-Seq

Purified RNA from cells cultured to mid-exponential phase in YPD at 30° C. was sequenced by SeqCenter (Pittsburgh, PA, USA). Library preparation was performed using the Stranded total RNA Prep Ligation with Ribo-Zero Plus kit (Illumina, San Diego, CA, USA) and 10 bp IDT for Illumina indices. Sequencing was performed using the NextSeq2000 platform to generate 2×50 bp reads. Sequencing data was analyzed using the Galaxy web platform public server at usegalaxy.org (70). Read quality was assessed using FastQC and reads were trimmed for quality using Cutadapt (75) with a Phred cutoff score of 20. Reads were then mapped to the C. auris B8441 reference assembly (NCBI GCA_002759435.2) using RNA Star (76) with the default parameters. Mapped reads were quantified using featureCounts (77) and differential expression was assessed using DESeq2 (78). Genes with a fold change greater than 2 times upregulated or downregulated and an adjusted p-value less than 0.05 were considered statistically significant.

### Dispersed Surface Flow Cytometry Adhesion Assay

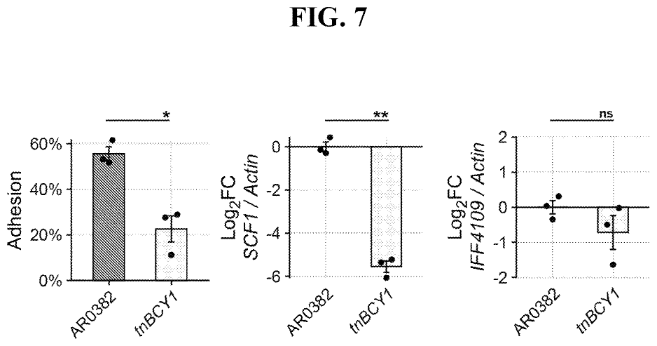

Adhesion to polystyrene was assessed using a previously established flow cytometric assay to measure the proportion of a population of cells able to attach to dispersed polystyrene surfaces in one hour (37). Cells from overnight culture were suspended in YPD containing green fluorescent polystyrene microspheres (1 μm, F-8823, Molecular Probes) at a ratio of 10 microspheres to 1 cell. For experiments involving additives, cells and microspheres were suspended in YPD containing the appropriate concentration of additive (NaCl or amino acids) and adjusted to neutral pH. This mixture was incubated with continuous inversion at 25 revolutions/min for 1 hour at room temperature. Cells were fixed with 4% formaldehyde for 10 minutes at room temperature, then washed and resuspended in PBS for flow cytometric analysis. Samples were analyzed using an LSRFortessa Flow Cytometer (BD Biosciences, NJ, USA) using a standard filter (FITC, 530/30). Acquisition settings were defined using the green fluorescent polystyrene microsphere samples to adjust the voltage of the fluorescent channel to the fourth logarithmic decade. Gating was performed as described in FIG. 7. FSC was used to gate cells from unattached microspheres. Data was collected for 10,000 gated events, which represented two distinct fluorescent populations. The percentage of cells with microspheres attached was determined by assessing the ratio of fluorescent to total events.

### High Throughput Adhesion Assay

An automated imaging-based assay was used for high throughput adhesion measurements. A total of 2,560 insertional mutants in the AR0382 strain background were arrayed and individually assayed in 96-well plates. Arrayed mutants were cultured from glycerol stocks on solid YPD agar at 30° C. The resulting colonies were used to seed 200 μL YPD cultures in 96 well plates and grown overnight at 30° C. For the adhesion assay, cells from each well were transferred into 100 μL YPD in a CellCarrier-96 Ultra Microplate with an optical cyclic olefin polymer surface (cat #NC1463153, Perkin Elmer, Waltham, MA, USA) using a 96-well microplate replicator (RePad 96 long pin, REP-001, Singer Instruments, United Kingdom). A single layer of visually distinct cells on the well surface were transferred. Each microplate contained 80 individual wells of mutants and 8 wells each of AR0382 and AR0387 as high adhesion and low adhesion controls, respectively. After adding cells, the plates were centrifuged at 210×g for 1 minute to settle the cells. Cells were incubated on the surface of the well for 1 hr at room temperature to allow for attachment. Unattached cells were removed by washing each well 3 times with 100 μL PBS and using a vacuum aspirator with 8 channel manifold (BrandTech QuickSip aspirator) to remove media between each wash. Each well was imaged in brightfield using a Yokogawa CellVoyager CQ1 automated microscope before and after washing. Four fields containing an average of approximately 1,000 cells/field were captured in defined positions such that the same four regions were imaged for each well before and after washing. Pre-processing of images was performed in Fiji ImageJ software (version 1.52) (79) using a custom macro that segmented cells in the brightfield images using edge detection and generated a binary mask from the segmented cells. The pre-processed images were quantified using CellProfiler software (version 3.1.9) (80) to generate a cell count for each captured field. A ratio of adhesive cells was calculated by dividing the count of the attached cells remaining after washing by the count of the input cells for each captured field.

The dynamic range of the assay was determined using a plate containing 48 wells of AR0382 and 48 wells of AR0387 as high and low adhesion controls, respectively. To determine the degree of separation between the two controls, the z-factor for the assay was calculated based on an established formula (81). Similarly, a z-factor was calculated based on the control wells for each mutant plate tested. The average z-factor for the control-only condition was 0.7167, indicating sufficient separation to detect differences between high and low adhesive strains, where an acceptance criteria of 0.5<z-factor<1.0 was established for each mutant plate based on the control wells for that plate.

A z-score was assessed for each mutant by subtracting the proportion of adhesive cells from the average proportion of adhesive cells amongst all the mutants and dividing by the standard deviation amongst all the mutants. Mutants with a z-score more negative than −3 were considered to have significantly reduced adhesion.

### Flocculation Assay

Cells were cultured overnight in YPD at 30° C. Cultures were vortexed to suspension for 10 sec at max speed, then placed upright at room temperature and allowed to settle for 20 min. Immediately after vortexing and at specified timepoints, a 20 μL aliquot was gently removed from the top of the culture and diluted to optical range in a 96 well plate. OD₆₀₀ was measured for each removed aliquot using a BioTek 800 TS absorbance reader. Flocculation activity was calculated as the percent reduction in OD₆₀₀ in aliquots at each timepoint compared to the initial reading.

### Immunofluorescence Microscopy and Flow Cytometry

Wild type and Ascf1+SCF1-FLAG cells were cultured overnight in YPD at 30° C. then harvested by centrifugation and washed in PBS. The cells were fixed with 4% formaldehyde for 10 min at room temperature, then washed and resuspended in PBS. Once fixed, the cells were blocked in 2% BSA for 60 min. The cells were then pelleted and resuspended in 0.1% BSA containing a 1:500 dilution of primary antibody (Rabbit α-FLAG Polyclonal, Sigma F7425) and incubated at room temperature for 3 hrs. The primary antibody was removed, and the cells were resuspended in 0.1% BSA containing a 1:500 dilution of secondary antibody (Goat α-Rabbit Igg, Alexafluor 594, Invitrogen A11037) and incubated at room temperature for 45 min, protected from light. The cells were washed three times in PBS with 0.1% Tween-20 and resuspended in PBS. Microscopy was performed using a Biotek Lionheart FX automated microscope using 100× oil objective (Olympus 1.4NA) and the TexasRed imaging filter cube and LED. Flow Cytometry was performed using an LSRFortessa Flow Cytometer (BD Biosciences, NJ, USA) using a standard filter (PE-Texas Red, 610/20). Data was collected for 50,000 events. Median fluorescence intensity was calculated using FlowJo™ v10.8.2 Software (BD Life Sciences).

### In Vitro Biofilm Formation

Cells were cultured overnight in YPD at 30° C. and resuspended in RPMI-1640 media. Each strain was standardized to a starting OD₆₀₀ of 0.5 in RPMI and seeded in 200 μL in 96-well plates with a virgin polystyrene surface. The seeded plates were sealed with a breathable membrane and incubated for 90 min at 37° C. with shaking at 250 rpm to allow for attachment. After this initial incubation, the media was aspirated and each well was washed with 200 μL PBS before replacing the liquid in each well with fresh RPMI-1640 media. The plates were sealed and biofilms were allowed to form for 24 hrs at 37° C. with constant orbital shaking at 250 rpm. After 24 hrs, each well was washed three times with 200 μL PBS. To quantify the oxidative activity of the viable biofilms, a colorimetric XTT reduction assay was performed (82). A 0.5 mg/mL XTT (XTT sodium salt, cat #AAJ61726MD, Thermo Scientific) solution in PBS was combined with a 0.32 mg/mL PMS (Phenazine Methosulfate, cat #10955, MP Biomedicals) solution in water at a 9:1 XTT:PMS ratio and 100 μL was added to each biofilm. The plate was incubated at 37° C. for 30 min before removing the media to a clean 96 well plate and measuring the OD₄₉₂. To image biofilms, the biofilms were fixed with 4% formaldehyde for 10 minutes at room temperature then washed two times with 200 μL PBS. The fixed biofilms were stained with calcofluor white (cat #18909, Sigma-Aldrich) for 10 minutes then washed three times with 200 μL PBS. Stained biofilms were imaged in Z-stacks using a Biotek Lionheart FX automated microscope at 400× magnification with the DAPI imaging filter cube and LED. Maximum intensity projections were generated using Gen5 software (version 3.12).

### Rat Catheter Biofilm Formation

In vivo biofilm testing was performed with a rat external jugular venous catheter model as previously described (83). Briefly, a 10⁶ cells/ml inoculum for each strain was allowed to grow on an internal jugular catheter placed in specific-pathogen-free Sprague-Dawley rats (16-week old, 400 g) for 24 h. After this period, biofilm formation on the intraluminal surface of the catheters was observed by scanning electron microscopy. Procedures were approved by the Institutional Animal Care and Use Committee at the University of Wisconsin, Madison (protocol MV1947).

### Scanning Electron Microscopy

SEM images were acquired on a ZEISS Gemini 450 scanning electron microscope using an accelerating voltage of 3.0 kV, a working distance of 6 mm, an Everhart-Thornley SE2 detector with optically coupled photomultiplier, and the ZEISS SmartSEM (v. 6.05) software.

### Microbial Attachment to Hydrocarbons (MATH) Assay

Cell surface hydrophobicity was determined using the microbial adhesion to hydrocarbon (MATH) assay (48), which measures the fraction of cells sequestered out of aqueous suspension by pure hydrocarbon. Overnight cultures were harvested by centrifugation and washed three times in PBS. The cells were standardized to an OD₆₀₀ of 0.4 in PBS using a Bio Tek 800 TS microplate reader. An aliquot of the cell suspension was reserved for an initial OD₆₀₀ reading (A0). 1200 μL of each cell suspension in PBS was transferred into clean, unused borosilicate glass test tubes with a 10 mm diameter. 200 μL n-hexadecane (Sigma-Aldrich, cat no: H6703) was gently overlaid atop the culture suspension in the test tube. The tubes were allowed to rest for 10 minutes at room temperature, then capped and vortexed at max speed for 1 min to mix the hydrocarbon and aqueous phases. The tubes were left for 15 minutes at room temperature to allow for separation of phases, after which an aliquot of the lower aqueous layer was taken for the final OD₆₀₀ reading (A1). The proportion of sequestered cells were calculated by dividing the final OD₆₀₀ reading (A1) by the initial reading (A0) according to the formula: [1−(A1/A0)]*100%.

### Plasma Etched Polystyrene Adhesion

Adhesion of C. auris cells to hydrophobic and hydrophilic substrates was modeled using untreated or surface-modified polystyrene multi-well plates (Nest Biotechnology Co., cat. 701311), which were considered to be hydrophobic as untreated (84). To assess adhesion to a hydrophilic substrate, the surface hydrophobicity of the polystyrene was modified by the addition of free radicals using vacuum plasma treatment. The plate was placed without the lid in a PE-25 plasma cleaner (Plasma Etch Inc, Nevada, USA) and treated for 90 seconds at 400W 50 kHz with 200 mtorr/27 Pa vacuum pressure. The plate was tested to be fully water-wettable by visual examination of a droplet spreading on the surface and compared to an untreated (hydrophobic) polystyrene plate. C. auris cells were cultured overnight in YPD at 30° C. and added to either the hydrophobic or hydrophilic polystyrene plates. Cells were centrifuged at 210×g for 1 min to gently settle a single layer of cells on the polystyrene surface. Plates were incubated for 1 hr at room temperature to allow for attachment. For each well, images of four fields in defined positions were taken in brightfield using a BioTek Lionheart FX automated microscope. Unattached cells were removed by washing 3 times with PBS, and fields in the same defined positions were imaged after washing. Cell counts for each field were determined using image analysis software as described above. The percentage of adherent cells was calculated by dividing the count of the attached cells remaining after washing by the total number of the cells input for each captured field.

### Statistics and Reproducibility

Statistical analyses were performed using R statistical software (version 4.0.3). Unless otherwise specified, experiments were performed in at least three independent biological replicates and data are presented as means±standard error of means from biological replicates, with each point representing individual biological replicates. Microscopy and photography images are representative of at least 3 experiments with similar results. Statistically significant differences were calculated using student's t-test or one-way ANOVA with Tukey's or Dunnett's post hoc test for multiple comparisons. Correlation was measured using Pearson correlation. *p≤0.05; **p≤0.01; ***p≤0.001; ns p>0.05.

### Results

C. auris Mediates Polymer Surface Attachment Through the Adhesin SCF1.

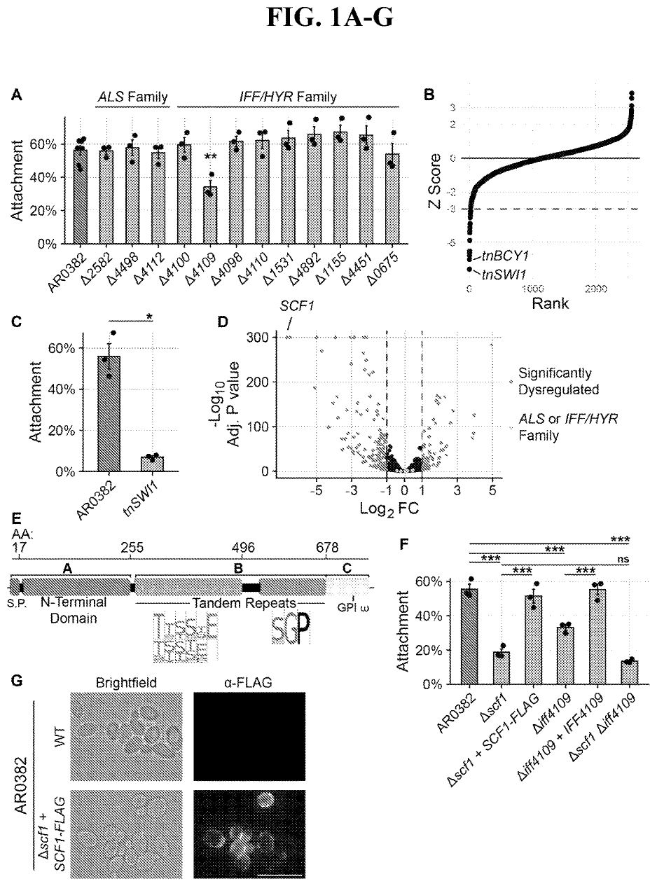

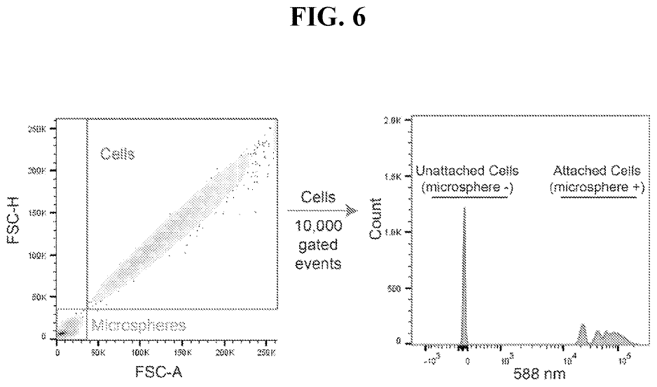

C. auris encodes twelve genes homologous to members of the characterized ALS and IFF/HYR adhesin families (24, 25, 27). To investigate the extent to which these adhesins mediate surface association, we generated individual deletion mutants in the clade I AR0382 background for each adhesin gene. We employed a flow cytometric adhesion assay to measure the ability of cells to attach to dispersed polystyrene microspheres in suspension as a model for surface association (FIG. 6) (28). We found that of the twelve adhesin mutants, only deletion of IFF4109 (B9J08_004109) conferred an adhesive defect, but this deletion failed to completely ablate attachment (FIG. 1A). To investigate the possibility that the residual attachment could be explained by occult adhesive factors, we screened a library of 2,560 arrayed insertional mutants for adhesion defects on a cyclic olefin surface, prioritizing mutants exhibiting the most significant defects (FIG. 1B). The greatest loss of adhesive capacity was observed in tnSWI1 (B9J08_003460) and tnBCY1 (B9J08_002818) mutants (FIG. 1B, FIG. 1C, FIG. 7). Compared to the AR0382 parent, the tnSWI1 mutant exhibited no significant transcriptional dysregulation of the ALS or IFF/HYR adhesins, suggesting alternative mediators of adhesion (FIG. 1D). The strongest, most significantly dysregulated gene in tnSWI1 was an uncharacterized ORF (B9J08_001458), which had no significant primary sequence homology to characterized genes (FIG. 1D). This gene, however, exhibited a putative three-domain architecture consistent with canonical GPI-anchored fungal adhesins (FIG. 1E) (23). Notably, this same gene was also strongly downregulated in the tnBCY1 mutant while IFF4109 was not (FIG. 7). Deletion of the B9J08_001458 ORF in AR0382 conferred a substantial adhesive defect, thus we refer to the gene as Surface Colonization Factor (SCF1) (FIG. 1F). The attachment defect could be rescued by complementation with an epitope-tagged SCF1 allele in the endogenous locus, and the epitope-tagged Scf1 protein localized to the cell surface, consistent with its role as an adhesin (FIG. 1F, FIG. 1G). Notably, deletion of IFF4109 in the Ascf1 background did not significantly reduce attachment beyond deletion of SCF1 alone, suggesting non-additive roles for these two adhesins (FIG. 1F).

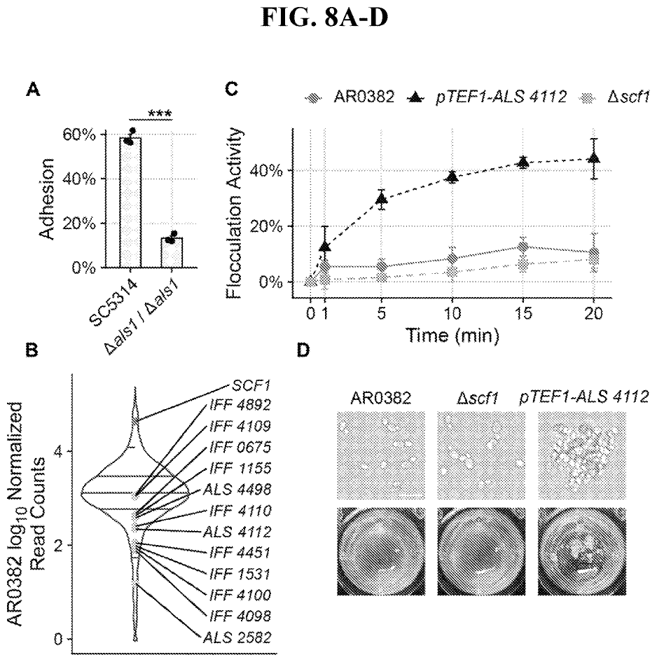

The specific reliance on SCF1 and IFF4109 for adhesion despite potential redundancy with other adhesins is reminiscent of other fungal pathogens. For instance, loss of ALS1 alone substantially reduces Candida albicans adhesion, despite the presence of seven other ALS genes (FIG. 8A) (29). In C. auris, adhesins exhibit structural and transcriptional variation, which may explain functional specificity (FIG. 8B) (25, 26). However, IFF4892 encodes the entire canonical adhesin architecture and shows similar transcript abundance to IFF4109, but of the two, only IFF4109 is required for adhesion, suggesting individual adhesins mediate specific adhesive mechanisms (FIG. 1A, FIG. 8B) (25). Such functional specificity is demonstrated by increased flocculation and aggregation associated with overexpression of ALS4112, while these phenotypes are not associated with SCF1 despite its transcriptional expression being in the top 2.5% of all genes in this strain background (FIG. 8B, FIG. 8C, FIG. 8D) (30, 31). Taken together, these findings suggest functional specificity for surface association for SCF1 and IFF4109.

C. auris Uniquely Relies on SCF1 for Adhesive Plasticity.

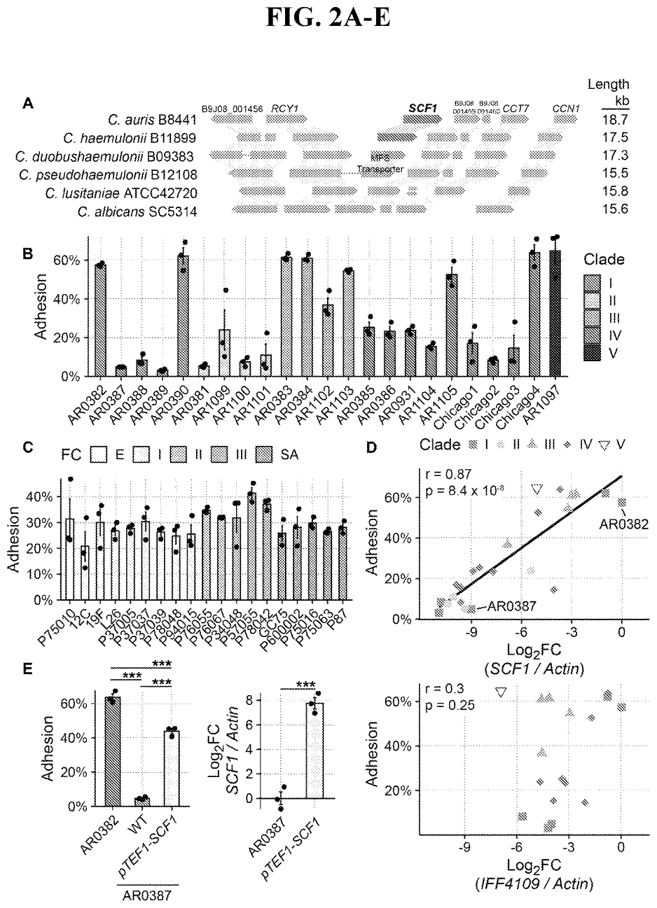

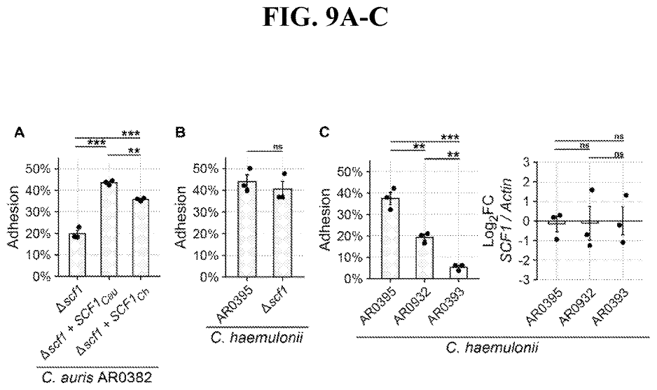

While many professional adhesins in Candida and Saccharomyces belong to conserved adhesin families across species, we identified homologs of SCF1 only in C. auris and the closely related Candida haemulonii species and not in other members of the haemulonii complex (FIG. 2A). Notably, SCF1 is encoded in a genomic locus in C. auris and C. haemulonii that is syntenic, absent an SCF1 homolog, even to distantly related species (FIG. 2A). Although the C. haemulonii SCF1 homolog was able to functionally complement Δscf1 in C. auris, it was not essential for adhesion in C. haemulonii and showed low expression across isolates, suggesting reliance on SCF1 for adhesion is unique to C. auris (FIG. 9).

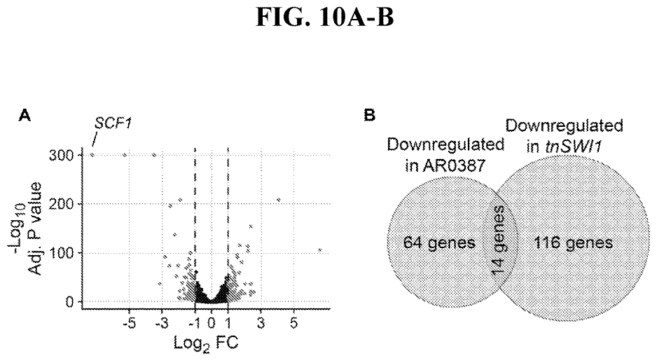

To investigate the generalizability of the reliance on SCF1 and the variability between C. auris strains, we measured adhesion for 23 C. auris isolates representing all five clades and diverse geographic origins of C. auris. These strains exhibited substantial adhesive variation, regardless of clade (p=2×10⁻¹⁶, F=35.06, one-way ANOVA) (FIG. 2B). In contrast, a similar analysis of 19 genetically diverse C. albicans clinical isolates demonstrated no significant adhesive variation (p=0.054, F=1.856, one-way ANOVA), suggesting C. auris has adapted plasticity around surface association that is not common to C. albicans (FIG. 2C). Interestingly, substantial variation in adherence was observed even between genetically similar isolates of C. auris, such as AR0382 and AR0387, which differ by only 206 coding SNPs (FIG. 2B). In the poorly adhesive AR0387, SCF1 was the most substantially down-regulated gene compared to the highly adhesive AR0382, reminiscent of the poorly adhesive tnSWI1 mutant (FIG. 10A, FIG. 1D). The transcriptome of AR0387 showed very little overlap with that of the tnSWI1 strain, however, suggesting that the dysregulation of SCF1 is not due to a defect in SW1/SNF complex activity in AR0387 (FIG. 10B). Furthermore, we did not observe any nucleotide variants in the SCF1 ORF or neighboring intragenic regions between AR0382 and AR0387.

To determine the extensibility of the association between SCF1 transcription and adhesion, we compared the adhesion and SCF1 transcript abundance for the entire panel of 23 C. auris isolates. Remarkably, transcript abundance of SCF1 was tightly positively correlated with adhesion, regardless of clade (r=0.87, p=8.4×10⁻⁸) (FIG. 2D). In contrast, we observed no association between transcriptional control of IFF4109 and adhesion (r=0.3, p=0.25) (FIG. 2D). Experimentally, transcriptional overexpression of SCF1 was sufficient to drive substantially increased adhesion in an otherwise poorly adhesive isolate, AR0387 (FIG. 2E). Importantly, the magnitude of overexpression using the TEF1 promoter (approximately 28-fold increase) was similar but did not exceed the naturally varying magnitude of expression difference in the two wild type isolates AR0382 and AR0387 (approximately 29-fold change) (FIG. 2D, FIG. 2E). These data suggest adhesive variation between C. auris isolates is associated with expression variation in SCF1.

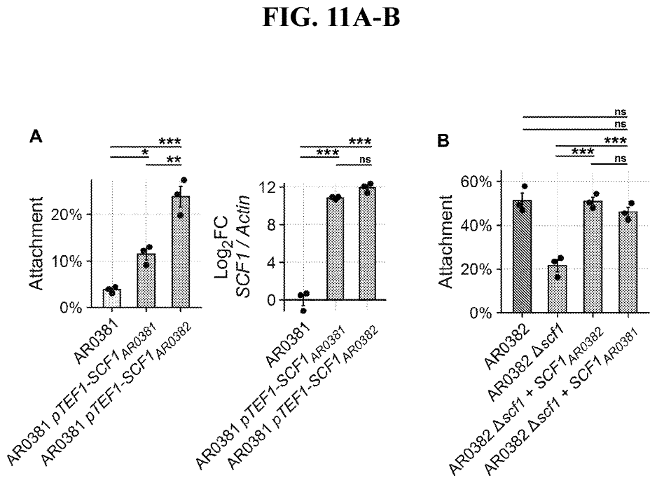

Beyond AR0382 and AR0387, where the SCF1 locus is invariant, different isolates exhibit allelic variation in SCF1 sequence, primarily concentrated in the repeat count and length of the low complexity tandem repeat domain (see table above). We therefore investigated whether allelic variation could also contribute to the adhesive variation exhibited by different isolates. Overexpression of the native SCF1 allele in AR0381, a poorly adhesive clade II isolate, was sufficient to increase attachment (FIG. 11A). However, overexpression of the clade I SCF1 allele from AR0382 resulted in a further significant increase in adhesion, despite similar levels of overexpression (FIG. 11A). Interestingly, in the clade I AR0382 background, which relies strongly on SCF1 for adhesion, complementation of the Ascf1 mutant with either the clade I or the clade II SCF1 allele resulted in similar levels of rescue of the adhesive phenotype (FIG. 11B). These findings suggest the sequence variation between these two SCF1 alleles does not intrinsically contribute to functional differences in adhesion, and that other factors also influence adhesive capacity.

### SCF1 and IFF4109 Mediate Surface Association Through Distinct Nonspecific Mechanisms.

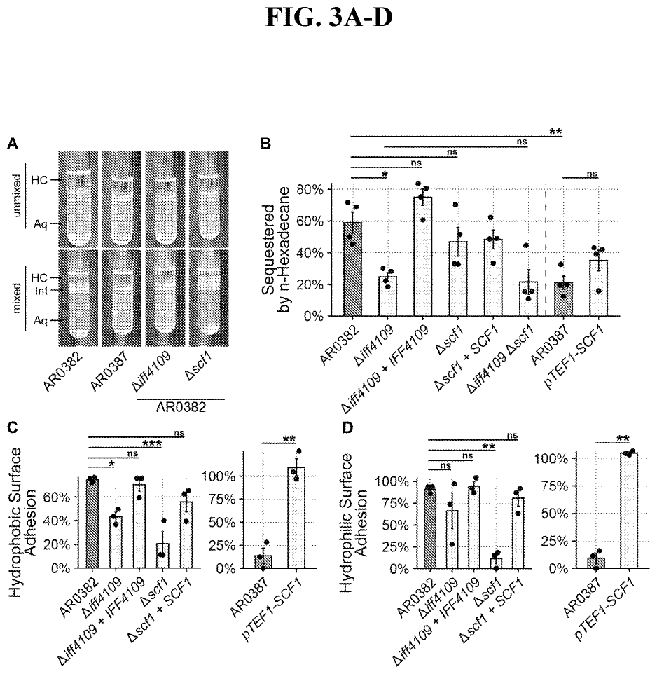

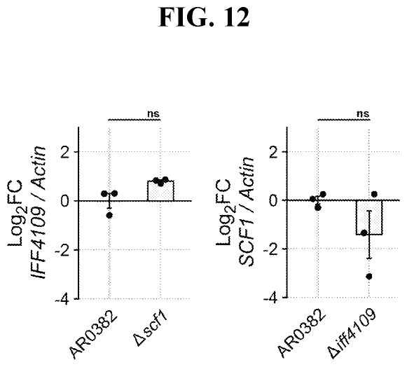

The reliance on SCF1 for surface association is complicated by the genetic interaction with IFF4109, where deletion of both does not result in a more severe adhesive defect than deletion of SCF1 alone (FIG. 1F). Loss of one adhesin did not result in dysregulation of the other, suggesting the interaction is not a regulatory one (FIG. 12). An alternative possibility is that the two genes contribute to the common adhesive phenotype through distinct mechanisms which may be alleviating in nature. For Candida species, adhesion to abiotic substrates is often nonspecific, with adhesins promoting a hydrophobic cell surface to drive affinity for hydrophobic substrates (32-34). In line with this, the highly adhesive AR0382 strain exhibited a high degree of cell surface hydrophobicity (CSH), while the poorly adhesive AR0387 strain exhibited low CSH (FIG. 3A, FIG. 3B). Deletion of the canonical IFF4109 in AR0382 resulted in reduced CSH, which could be rescued to wild type levels by complementation (FIG. 3A, FIG. 3B). However, deletion or overexpression of SCF1 did not significantly impact CSH in AR0382 or AR0387 (FIG. 3A, FIG. 3B).

Adhesins that promote cell surface hydrophobicity likewise promote affinity for hydrophobic substrates (33). We measured the adhesion of C. auris isolates to both an untreated hydrophobic polystyrene surface and a polystyrene surface modified using a vacuum plasma treatment to become strongly hydrophilic. Both IFF4109 and SCF1 mediated adhesion to the hydrophobic substrate (FIG. 3C). However, only SCF1, and not IFF4109, mediated adhesion to the hydrophilic substrate, suggesting the adhesive mechanism for SCF1 is not dependent on hydrophobicity (FIG. 3D). Notably, AR0382 and AR0387 still exhibited differential adhesion to the hydrophilic surface, indicating hydrophobic interactions are not primarily responsible for the differential strain phenotypes (FIG. 3D).

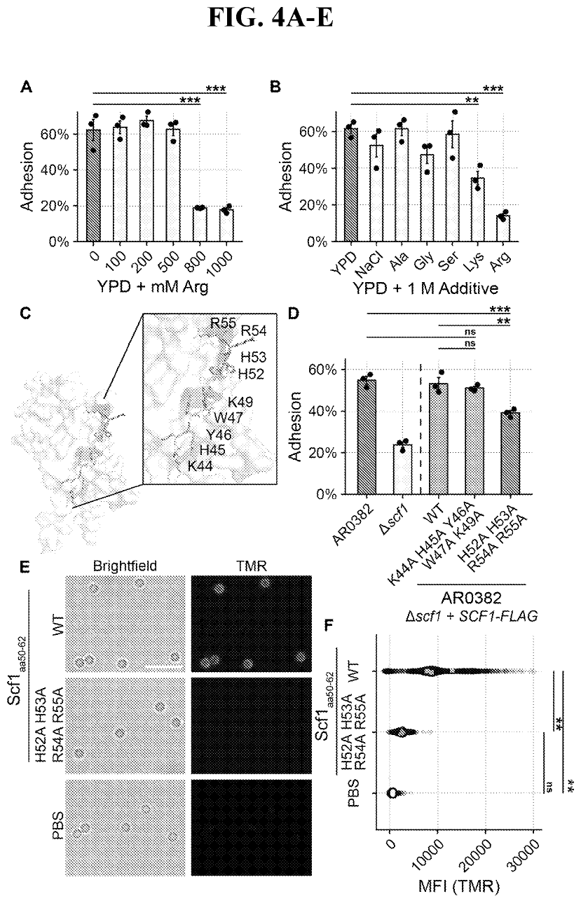

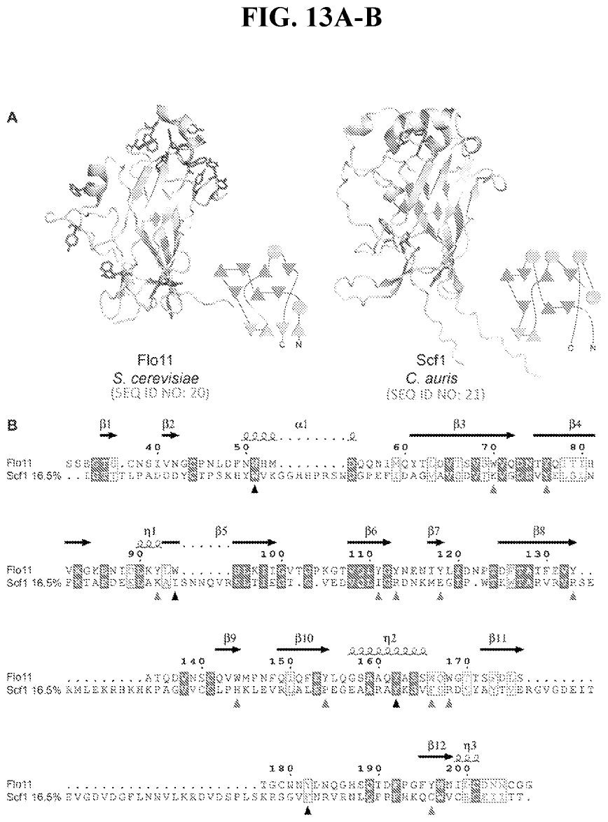

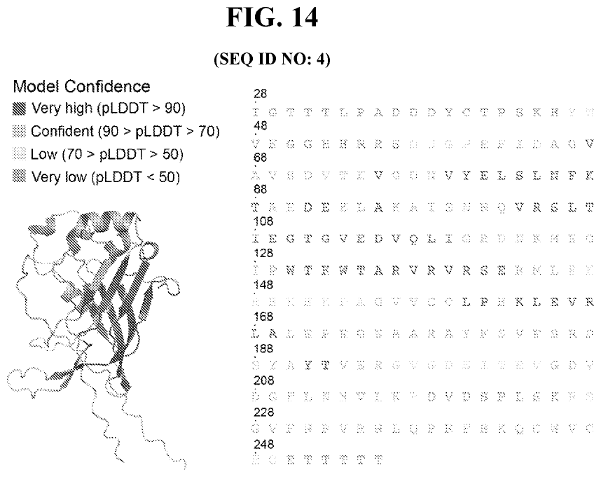

To investigate the mechanism of Scf1 adhesion, we examined the apical N-terminal domain using AlphaFold2, which suggests this domain contains a core Fibronectin-type 111 fold, similar to the FLO11 family of adhesins characterized in Saccharomyces cerevisiae and conserved throughout Ascomycota (FIG. 13A) (32, 35). However, SCF1 does not exhibit significant primary sequence homology to S. cerevisiae FLO11 and lacks conservation of the canonical aromatic bands responsible for adhesive functions in true FLO11 homologs (FIG. 13A, FIG. 13B) (32, 35). Furthermore, model confidence dwindles outside the Fibronectin fold, suggesting substantial variation from Flo11 adhesins (FIG. 14). At the primary sequence level, the SCF1 N-terminal domain exhibits an enrichment of arginine and lysine residues not present in other characterized yeast adhesin families (see table above). Adhesive systems in many marine organisms rely on similarly cation-rich proteins, which mediate adhesion through displacement of hydrated ions at the surface liquid interface and direct cation-7t interactions between adhesion proteins and substrates (36-40). We reasoned that if Scf1 relied on such interactions, adhesion could be inhibited by a saturating concentration of cations at the substrate interface that could not be competitively displaced by SCF1. Consistent with this hypothesis, high concentrations of arginine in solution were sufficient to ablate AR0382 adhesion (FIG. 4A). Similar concentrations of NaCl or other non-cationic amino acids did not produce the same effect, while exogenous lysine produced a more modest inhibition of attachment, consistent with lysine's weaker ability to form electrostatic interactions (FIG. 4B) (41).

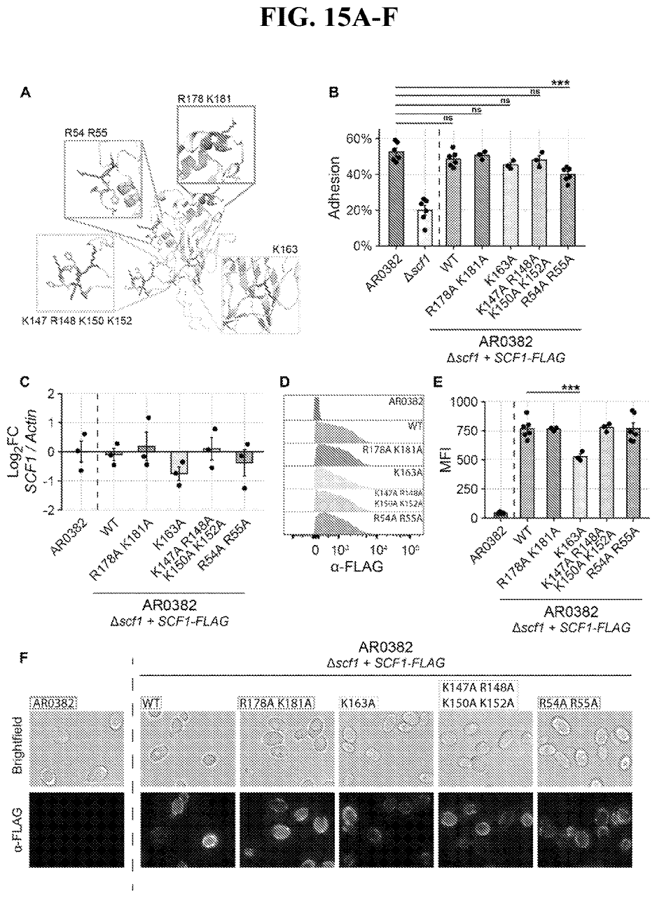

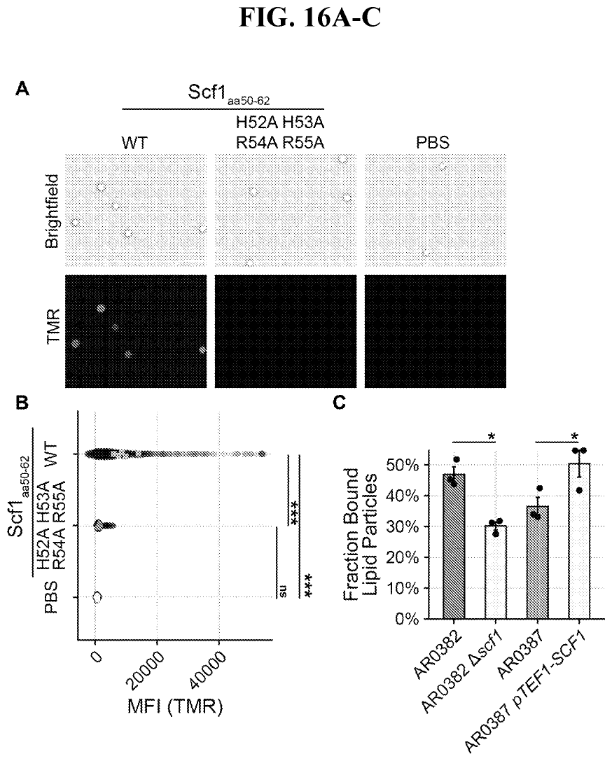

Given the abundance of cationic residues in the Scf1 N-terminal domain, we next investigated whether specific regions or residues were critical for Scf1 activity. We generated point mutations in cationic residues in different regions of the domain, focusing on residues that clustered with aromatic groups, as this pattern potentiates electrostatic adhesion (FIG. 15A) (42). Several mutations had no adhesive impact, but an R54A R55A mutant exhibited a modest adhesive defect, while demonstrating no discernable defect in SCF1 transcription, protein expression, or localization (FIG. 15). Mutating the entire cation-aromatic cluster, H52 H53 R54 R55, resulted in a similar adhesive defect (FIG. 4C, FIG. 4D, FIG. 16). Notably, a nearby cation-aromatic cluster, K44-K49, which was predicted to be less surface exposed, was not required for adhesion (FIG. 4C, FIG. 4D, FIG. 16). To determine whether surface exposure of the HHRR cluster (res. 52-55) would be sufficient to promote adhesion, we synthesized peptides corresponding to Scf1 residues 50-62 with the intact wild-type cluster or the HHRR residues mutated. The wild-type peptide adhered to polystyrene microspheres, but mutation of the HHRR cluster completely ablated this ability (FIG. 4E, FIG. 4F). Interestingly, similar patterns of cation-aromatic clusters are also abundant in some lipid-binding proteins (40, 43). The wild type Scf1 peptide was similarly able to adhere to phosphotidylcholine microparticles, and SCF1 expression potentiated lipid particle binding by C. auris cells, suggesting SCF1 may also contribute to association with biotic surfaces (FIG. 17).

### SCF1 Promotes Long-Term Colonization and Virulence Phenotypes.

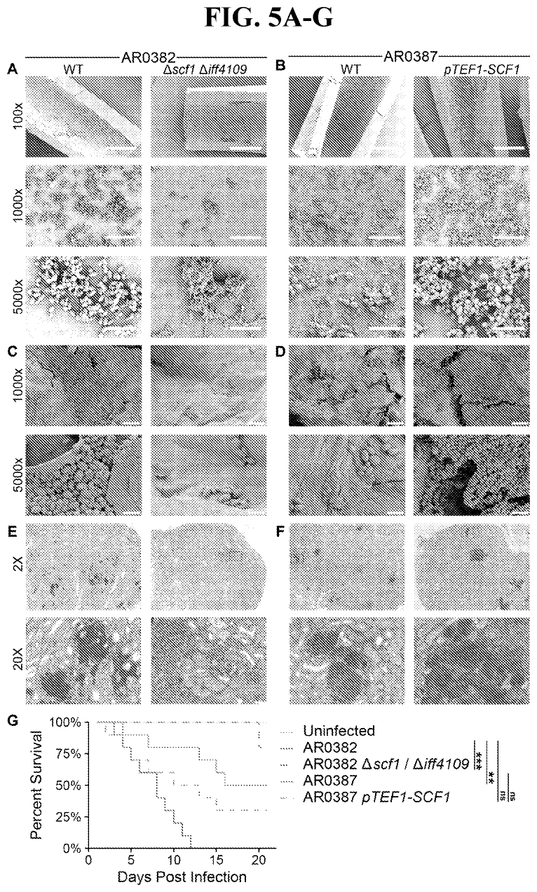

We next investigated the impact of SCF1 and IFF4109 on colonization phenotypes beyond initial surface association. We first measured the importance of these adhesins for biofilm growth, which can promote prolonged environmental persistence (11, 44-46). The two adhesins were functionally redundant, and deletion of both was required to ablate biofilm formation in AR0382, suggesting the partial adhesive contributions of each is sufficient to establish colonization (FIG. 17A). Importantly though, expression of SCF1 alone in otherwise biofilm-incompetent isolates was sufficient to establish biofilm colonization (FIG. 17B, FIG. 17C, FIG. 17D, FIG. 17E). This pattern continued when investigating in vivo biofilms, where loss of SCF1 and IFF4109 ablated the ability of AR0382 to colonize the luminal surface of an in vivo polyethylene rat central venous catheter, and overexpression of SCF1 was sufficient to potentiate colonization in AR0387 (FIG. 5A, FIG. 5B).

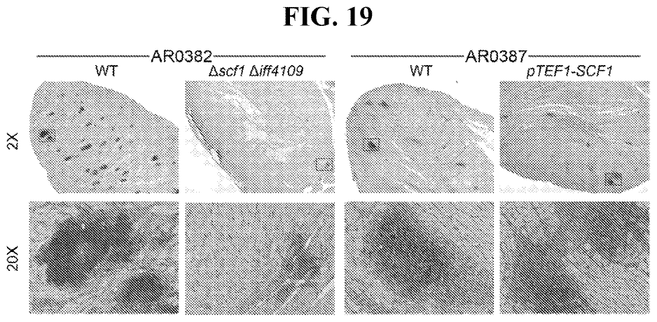

We then investigated whether biological surface association followed the same reliance on these adhesins. Again, we observed that loss of SCF1 and IFF4109 diminished the ability of AR0382 to colonize ex vivo human skin explants and in vivo murine skin, while overexpression of SCF1 potentiated skin colonization by AR0387 (FIG. 5C, FIG. 5D, FIG. 19). Given this potential for interaction with host tissues, we also investigated the importance of these adhesins in hematogenously disseminated infection. Histopathological examination of tissues collected from mice 7 days after intravenous C. auris infection revealed that loss of SCF1 and IFF4109 reduced AR0382 dissemination to the kidneys and heart, while overexpression of SCF1 in AR0387 was sufficient to increase fungal lesions (FIG. 5E, FIG. 5F, FIG. 19). Loss of SCF1 and IFF4109 substantially attenuated the virulence of AR0382, with the wild type causing 100% mortality within 12 days of infection and the mutant causing only 20% mortality after 21 days (FIG. 5G). Similarly, overexpression of SCF1 reduced the median survival of mice infected with AR0387 from 18.5 days to 11.5 days and ablated the difference in overall survival between the less virulent AR0387 and the more virulent AR0382 (FIG. 5G).

C. auris encodes adhesins sharing sequence similarity with the conserved ALS and IFF/HYR adhesin families, and proposed models suggest differential utilization of these adhesins may contribute to epidemiological differences amongst isolates (25, 27). Our findings suggest the C. auris-specific adhesin SCF1 and the conserved adhesin IFF4109 are the principal mediators of association with abiotic surfaces, and additionally contribute substantially to infection and long-term colonization of both biological and abiotic surfaces. Notably, we observed widespread differential regulation of SCF1 among C. auris isolates regardless of clade, suggesting transcriptional control of this adhesin has adapted more recently than clade separation. The widespread plasticity around a single genetic element responsible for diverse clinically-relevant phenotypes poses the potential for substantial impact in outbreak settings.

Prior work suggests vaccination or monoclonal antibody therapy targeting Als or IFF/HYR adhesins offer protection against lethal C. auris infection (27, 47). Furthermore, the complementary function of SCF1 and IFF4109 with divergent mechanisms suggests C. auris has evolved the capacity for promiscuous surface association, which likely contributes to the ability of this pathogen to readily colonize medically relevant substrates. Mediation of hydrophobic interactions is largely conserved among fungal adhesins, consistent with our observations that IFF4109 controls attachment through this mechanism (32-34). The cation rich SCF1, however, appears to more closely functionally resemble proteins from bivalve, barnacle, and Vibrio adhesion systems. For these organisms, cation-dependent surface interactions promote adhesion in aqueous and highly ionic environments (36-39). C. auris has been isolated from the coastal wetlands of the Andaman Islands in the Indian Ocean and from a Colombian estuary, suggesting its natural habitat may be marine in nature and this ecological niche may have conferred similar selective pressures on adhesion mechanisms (48, 49). Development of unique adhesion biology may in part explain the tenacity of this organism on medically-relevant substrates.

### REFERENCES

1. Chakrabarti and Sood, On the emergence, spread and resistance of Candida auris: host, pathogen and environmental tipping points. J. Med. Microbiol. (2021).
2. Akinbobola, et al., Environmental reservoirs of the drug-resistant pathogenic yeast Candida auris. PLoS Pathog. 19, e1011268 (2023).
3. Ashkenazi-Hoffnung, and Rosenberg Danziger, Navigating the New Reality: A Review of the Epidemiological, Clinical, and Microbiological Characteristics of Candida auris, with a Focus on Children. J Fungi (Basel). 9 (2023).
4. Chow, et al., US Candida auris Investigation Team, Multiple introductions and subsequent transmission of multidrug-resistant Candida auris in the USA: a molecular epidemiological survey. Lancet Infect. Dis. 18, 1377-1384 (2018).
5. S. C. Roberts, T. R. Zembower, E. A. Ozer, C. Qi, Genetic Evaluation of Nosocomial Candida auris Transmission. J. Clin. Microbiol. (2021).
6. Vallabhaneni et al., Investigation of the First Seven Reported Cases of Candida auris, a Globally Emerging Invasive, Multidrug-Resistant Fungus—United States, May 2013-August 2016. MMWR Morb. Mortal. Wkly. Rep. 65, 1234-1237 (2016).
7. WHO fungal priority pathogens list to guide research, development and public health action. Geneva: World Health Organization (2022).
8. Sexton et al., Positive correlation between Candida auris skin-colonization burden and environmental contamination at a ventilator-capable skilled nursing facility in Chicago. Clin. Infect. Dis. (2021).
9. Proctor et al., Integrated genomic, epidemiologic investigation of Candida auris skin colonization in a skilled nursing facility. Nat. Med. (2021).
10. Adams et al., Candida auris Investigation Workgroup, Candida auris in Healthcare Facilities, New York, USA, 2013-2017. Emerg. Infect. Dis. 24, 1816-1824 (2018).
11. Oremefetse et al., Survival of Candida auris on environmental surface material and low-level resistance to disinfectant. J. Hosp. Infect. (2023).
12. Welsh et al., Survival, Persistence, and Isolation of the Emerging Multidrug-Resistant Pathogenic Yeast Candida auris on a Plastic Health Care Surface. J. Clin. Microbiol. 55, 2996-3005 (2017).
13. de Almeida Jr, et al., The Candida auris Brazilian Study Group, Axillary Digital Thermometers uplifted a multidrug-susceptible Candida auris outbreak among COVID-19 patients in Brazil. Mycoses (2021).
14. Eyre, et al., Candida auris Outbreak and Its Control in an Intensive Care Setting. N. Engl. J. Med. 379, 1322-1331 (2018).
15. Patterson et al., Cloth Lanyards as a Source of Intermittent Transmission of Candida auris on an ICU. Crit. Care Med. 49, 697-701 (2021).
16. Kurt et al., Candida auris Fungemia and a local spread taken under control with infection control measures: First report from Turkey. Indian J. Med. Microbiol. (2021).
17. Hinrichs, et al., Successful control of Candida auris transmission in a German COVID-19 intensive care unit. Mycoses (2022), doi:10.1111/myc.13443.
18. Vila et al., Comparative Evaluations of the Pathogenesis of Candida auris Phenotypes and Candida albicans Using Clinically Relevant Murine Models of Infections. mSphere. 5 (2020).
19. Vinayagamoorthy et al., Prevalence, risk factors, treatment and outcome of multidrug resistance Candida auris infections in Coronavirus disease (COVID-19) patients: A systematic review. Mycoses. 65, 613-624 (2022).
20. Khojasteh et al., Candida auris and COVID-19: A health threatening combination. Curr Med Mycol. 8, 44-50 (2022).
21. E. Rajni, A. Jain, S. Gupta, Y. Jangid, R. Vohra, Risk Factors for Candidemia in Intensive Care Unit: A Matched Case Control Study from North-Western India. Acta Medica. 65, 83-88 (2022).
22. Allaw et al., COVID-19 and C. auris: A Case-Control Study from a Tertiary Care Center in Lebanon. Microorganisms. 10 (2022).
23. Lyman et al., Worsening Spread of Candida auris in the United States, 2019 to 2021. Ann. Intern. Med. (2023).
24. Centers for Disease Control and Prevention, “Infection Prevention and Control for Candida auris” (2023).
25. Bishop, et al., Guidance for the laboratory investigation, management and infection prevention and control of cases of Candida auris. Public Health England (2017) (available at https://assets.publishing.service.gov.uk/government/uploads/system/uploads/attachment_data/file/637685/Updated_Candida_auris_Guidance_v2.pdf).
26. A. M. Aldejohann, M. Wiese-Posselt, P. Gastmeier, O. Kurzai, Expert recommendations for prevention and management of Candida auris transmission. Mycoses (2022), doi:10.1111/myc.13445.
27. Huang et al., Murine model of colonization with fungal pathogen Candida auris to explore skin tropism, host risk factors and therapeutic strategies. Cell Host Microbe (2020).
28. Eix et al., Ex vivo human and porcine skin effectively model C. auris colonization, differentiating robust and poor fungal colonizers. J. Infect. Dis. (2022).
29. M. V. Horton, C. J. Johnson, J. F. Kernien, T. D. Patel, B. C. Lam, J. Z. A. Cheong, J. J. Meudt, D. Shanmuganayagam, L. R. Kalan, J. E. Nett, Candida auris Forms High-Burden Biofilms in Skin Niche Conditions and on Porcine Skin. mSphere. 5 (2020).
30. P. W. J. de Groot, O. Bader, A. D. de Boer, M. Weig, N. Chauhan, Adhesins in human fungal pathogens: glue with plenty of stick. Eukaryot. Cell. 12, 470-481 (2013).
31. P. N. Lipke, P. Ragonis-Bachar, Sticking to the Subject: Multifunctionality in Microbial Adhesins. J Fungi (Basel). 9 (2023).
32. L.-O. Essen, M. S. Vogt, H.-U. Masch, Diversity of GPI-anchored fungal adhesins. Biol. Chem. 401, 1389-1405 (2020).
33. J. F. Munoz, R. M. Welsh, T. Shea, D. Batra, L. Gade, D. Howard, L. A. Rowe, J. F.

Meis, A. P. Litvintseva, C. A. Cuomo, Clade-specific chromosomal rearrangements and loss of subtelomeric adhesins in Candida auris. Genetics (2021).
34. R. A. Smoak, L. F. Snyder, J. S. Fassler, B. Z. He, Parallel Expansion and Divergence of an Adhesin Family in Pathogenic Yeasts. Genetics (2023).
35. Oh, et al., Using Genomics to Shape the Definition of the Agglutinin-Like Sequence (ALS) Family in the Saccharomycetales. Front. Cell. Infect. Microbiol. 11, 794529 (2021).
36. S. Singh, P. Uppuluri, Z. Mamouei, A. Alqarihi, H. Elhassan, S. French, S. R.

Lockhart, T. Chiller, J. E. Edwards Jr, A. S. Ibrahim, The NDV-3A vaccine protects mice from multidrug resistant Candida auris infection. PLoS Pathog. 15, e1007460 (2019).
37. A. Silva-Dias, I. M. Miranda, R. Rocha, M. Monteiro-Soares, A. Salvador, A. G. Rodrigues, C. Pina-Vaz, A novel flow cytometric protocol for assessment of yeast cell adhesion. Cytometry A. 81, 265-270 (2012).
38. D. J. Santana, T. R. O'Meara, Forward and reverse genetic dissection of morphogenesis identifies filament-competent Candida auris strains. Nat. Commun. 12, 7197 (2021).
39. J. S. Finkel, W. Xu, D. Huang, E. M. Hill, J. V. Desai, C. A. Woolford, J. E. Nett, H.

Taff, C. T. Norice, D. R. Andes, F. Lanni, A. P. Mitchell, Portrait of Candida albicans adherence regulators. PLoS Pathog. 8, e1002525 (2012).
40. L. van Wijlick, S. Znaidi, A. Hernández-Cervantes, V. Basso, S. Bachellier-Bassi, C. d'Enfert, Functional Portrait of Irf1 (Orf19.217), a Regulator of Morphogenesis and Iron Homeostasis in Candida albicans. Front. Cell. Infect. Microbiol. 12, 960884 (2022).
41. C. Pelletier, A. J. P. Brown, A. Lorenz, Candida auris undergoes adhesin-dependent and -independent cellular aggregation. bioRxiv (2023), p. 2023.04.21.537817.
42. J. Bing, Z. Guan, T. Zheng, Z. Zhang, S. Fan, C. L. Ennis, C. J. Nobile, G. Huang, Clinical isolates of Candida auris with enhanced adherence and biofilm formation due to genomic amplification of ALS4. PLoS Pathog. 19, e1011239 (2023).
43. S.-H. Oh, G. Cheng, J. A. Nuessen, R. Jajko, K. M. Yeater, X. Zhao, C. Pujol, D. R. Soll, L. L. Hoyer, Functional specificity of Candida albicans Als3p proteins and clade specificity of ALS3 alleles discriminated by the number of copies of the tandem repeat sequence in the central domain. Microbiology. 151 (2005), pp. 673-681.
44. R. Mani, R. P. St Onge, J. L. Hartman 4th, G. Giaever, F. P. Roth, Defining genetic interaction. Proc. Natl. Acad. Sci. U.S.A 105, 3461-3466 (2008).
45. T. Kraushaar, S. Bruckner, M. Veelders, D. Rhinow, F. Schreiner, R. Birke, A. Pagenstecher, H.-U. Masch, L.-O. Essen, Interactions by the Fungal Flo11 Adhesin Depend on a Fibronectin Type III-like Adhesin Domain Girdled by Aromatic Bands. Structure. 23, 1005-1017(2015).
46. S. El-Kirat-Chatel, A. Beaussart, S. Derclaye, D. Alsteens, S. Kucharikovi, P. Van Dijck, Y. F. Dufrene, Force Nanoscopy of Hydrophobic Interactions in the Fungal Pathogen Candida glabrata. ACS Nano. 9 (2015), pp. 1648-1655.
47. C. Valotteau, V. Prystopiuk, B. P. Cormack, Y. F. Dufrene, Atomic Force Microscopy Demonstrates that Candida glabrata Uses Three Epa Proteins To Mediate Adhesion to Abiotic Surfaces. mSphere. 4 (2019).
48. M. Rosenberg, D. Gutnick, E. Rosenberg, Adherence of bacteria to hydrocarbons: A simple method for measuring cell-surface hydrophobicity. FEMS Microbiol. Lett. 9, 29-33 (1980).
49. C. J. van Oss, D. R. Absolom, A. W. Neumann, The “hydrophobic effect”: Essentially a van der Waals interaction. Colloid Polym. Sci. 258, 424-427 (1980).
50. S. Zheng, M. Bawazir, A. Dhall, H.-E. Kim, L. He, J. Heo, G. Hwang, Implication of Surface Properties, Bacterial Motility, and Hydrodynamic Conditions on Bacterial Surface Sensing and Their Initial Adhesion. Front Bioeng Biotechnol. 9, 643722 (2021).
51. J. N. Israelachvili, Intermolecular and Surface Forces (Elsevier, 30 Corporate Dr., Suite 400, Burlington, MA 01803, USA, 2011).
52. S. Bruckner, R. Schubert, T. Kraushaar, R. Hartmann, D. Hoffmann, E. Jelli, K. Drescher, D. J. Müller, L. Oliver Essen, H.-U. Mosch, Kin discrimination in social yeast is mediated by cell surface receptors of the FoI 1 adhesin family. Elife. 9 (2020).
53. G. P. Maier, M. V. Rapp, J. H. Waite, J. N. Israelachvili, A. Butler, BIOLOGICAL ADHESIVES. Adaptive synergy between catechol and lysine promotes wet adhesion by surface salt displacement. Science. 349, 628-632 (2015).
54. Y. Li, C. Liang, L. Gao, S. Li, Y. Zhang, J. Zhang, Y. Cao, Hidden complexity of synergistic roles of Dopa and lysine for strong wet adhesion. Mater. Chem. Front. 1, 2664-2668 (2017).
55. C. Liang, J. Strickland, Z. Ye, W. Wu, B. Hu, D. Rittschof, Biochemistry of Barnacle Adhesion: An Updated Review. Frontiers in Marine Science. 6 (2019).
56. S. Kim, H. Y. Yoo, J. Huang, Y. Lee, S. Park, Y. Park, S. Jin, Y. M. Jung, H. Zeng, D. S. Hwang, Y. Jho, Salt Triggers the Simple Coacervation of an Underwater Adhesive When Cations Meet Aromatic 11 Electrons in Seawater. ACS Nano. 11, 6764-6772 (2017).
57. S. Sokalingam, G. Raghunathan, N. Soundrarajan, S.-G. Lee, A study on the effect of surface lysine to arginine mutagenesis on protein stability and structure using green fluorescent protein. PLoS One. 7, e40410 (2012).
58. H. Fan, J. Wang, Z. Tao, J. Huang, P. Rao, T. Kurokawa, J. P. Gong, Adjacent cationic-aromatic sequences yield strong electrostatic adhesion of hydrogels in seawater. Nat. Commun. 10, 5127 (2019).
59. R. Kean, E. McKloud, E. M. Townsend, L. Sherry, C. Delaney, B. L. Jones, C. Williams, G. Ramage, The comparative efficacy of antiseptics against Candida auris biofilms. Int. J. Antimicrob. Agents. 52, 673-677 (2018).
60. R. Kean, C. Delaney, L. Sherry, A. Borman, E. M. Johnson, M. D. Richardson, R. Rautemaa-Richardson, C. Williams, G. Ramage, Transcriptome Assembly and Profiling of Candida auris Reveals Novel Insights into Biofilm-Mediated Resistance. mSphere. 3 (2018), doi:10.1128/mSphere.00334-18.
61. B. Short, J. Brown, C. Delaney, L. Sherry, C. Williams, G. Ramage, R. Kean, Candida auris exhibits resilient biofilm characteristics in vitro: implications for environmental persistence. J. Hosp. Infect. 103, 92-96 (2019).
62. S. Allert, D. Schulz, P. Kammer, P. GroBmann, T. Wolf, S. Schauble, G. Panagiotou, S. Brunke, B. Hube, From environmental adaptation to host survival: Attributes that mediate pathogenicity of Candida auris. Virulence. 13, 191-214 (2022).
63. R. A. Smoak, L. F. Snyder, J. S. Fassler, B. Z. He, Parallel Expansion and Divergence of an Adhesin Family in Pathogenic Yeasts Including Candida auris. bioRxiv (2022), p. 2022.02.09.479577.
64. S. McLaughlin, J. Wang, A. Gambhir, D. Murray, PIP2 and Proteins: Interactions, Organization, and Information Flow (2003),doi:10.1146/annurev.biophys.31.082901.134259.
65. P. Arora, P. Singh, Y. Wang, A. Yadav, K. Pawar, A. Singh, G. Padmavati, J. Xu, A. Chowdhary, Environmental Isolation of Candida auris from the Coastal Wetlands of Andaman Islands, India. MBio. 12 (2021).
66. P. Escand6n, Novel Environmental Niches for Candida auris: Isolation from a Coastal Habitat in Colombia. J Fungi (Basel). 8 (2022).
67. J. D. Lutgring, M.-J. Machado, F. H. Benahmed, P. Conville, R. M. Shawar, J. Patel, A. C. Brown, FDA-CDC Antimicrobial Resistance Isolate Bank: a Publicly Available Resource To Support Research, Development, and Regulatory Requirements. J. Clin. Microbiol. 56, e01415-17 (2018).
68. A. O. Veri, Z. Miao, R. S. Shapiro, F. Tebbji, T. R. O'Meara, S. H. Kim, J. Colazo, K. Tan, V. K. Vyas, M. Whiteway, N. Robbins, K. H. Wong, L. E. Cowen, Tuning Hsfl levels drives distinct fungal morphogenetic programs with depletion impairing Hsp90 function and overexpression expanding the target space. PLoS Genet. 14, e1007270 (2018).
69. S. O. Park, C. Frazer, R. J. Bennett, An Adjuvant-Based Approach Enables the Use of Dominant HYG and KAN Selectable Markers in Candida albicans. mSphere. 7, e0034722 (2022).
70. Afgan et al., The Galaxy platform for accessible, reproducible and collaborative biomedical analyses: 2018 update. Nucleic Acids Res. 46, W537-W544 (2018).
71. A. M. Bolger, M. Lohse, B. Usadel, Trimmomatic: a flexible trimmer for Illumina sequence data. Bioinformatics. 30, 2114-2120 (2014).
72. H. Li, R. Durbin, Fast and accurate short read alignment with Burrows-Wheeler transform. Bioinformatics. 25 (2009), pp. 1754-1760.
73. D. W. Lee, C. P. Hong, H. A. Kang, An effective and rapid method for RNA preparation from non-conventional yeast species. Anal. Biochem. 586, 113408 (2019).
74. J. Ye, G. Coulouris, I. Zaretskaya, I. Cutcutache, S. Rozen, T. L. Madden, Primer-BLAST: a tool to design target-specific primers for polymerase chain reaction. BMC Bioinformatics. 13, 134 (2012).
75. M. Martin, Cutadapt removes adapter sequences from high-throughput sequencing reads. EMBnet.journal. 17, 10-12 (2011).
76. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics. 29, 15-21 (2013).
77. Y. Liao, G. K. Smyth, W. Shi, featureCounts: an efficient general purpose program for assigning sequence reads to genomic features. Bioinformatics. 30, 923-930 (2014).
78. M. I. Love, W. Huber, S. Anders, Moderated estimation of fold change and dispersion for RNA-seq data with DESeq2. Genome Biol. 15, 550 (2014).
79. J. Schindelin, I. Arganda-Carreras, E. Frise, V. Kaynig, M. Longair, T. Pietzsch, S. Preibisch, C. Rueden, S. Saalfeld, B. Schmid, J.-Y. Tinevez, D. J. White, V. Hartenstein, K. Eliceiri, P. Tomancak, A. Cardona, Fiji: an open-source platform for biological-image analysis. Nat. Methods. 9, 676-682 (2012).
80. M. R. Lamprecht, D. M. Sabatini, A. E. Carpenter, CellProfiler™: free, versatile software for automated biological image analysis. Biotechniques. 42, 71-75 (2007).
81. M.-A. Bray, A. Carpenter, Imaging Platform, Broad Institute of MIT and Harvard, “Advanced Assay Development Guidelines for Image-Based High Content Screening and Analysis” in Assay Guidance Manual, Eds. (Eli Lilly & Company and the National Center for AdvancingTranslational Sciences, Bethesda (MD), 2017).
82. Gulati et al., In Vitro Culturing and Screening of Candida albicans Biofilms. Curr. Protoc. Microbiol. 50, e60 (2018).
83. Andes, et al., Development and characterization of an in vivo central venous catheter Candida albicans biofilm model. Infect. Immun. 72, 6023-6031 (2004).
84. Li et al., Contact angle of water on polystyrene thin films: effects of CO(2) environment and film thickness. Langmuir. 23, 9785-9793 (2007).
85. Pacilli, et al., Regional Emergence of Candida auris in Chicago and Lessons Learned From Intensive Follow-up at 1 Ventilator-Capable Skilled Nursing Facility. Clin. Infect. Dis. 71, e718-e725 (2020).

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising:
an engineered polynucleotide encoding at least a portion of Surface Colonization Factor (SCF1) from Candida auris,
wherein the polynucleotide is at least partially optimized for enhanced expression, productive co-translational protein folding, increased stability, or a combination thereof.

2. The composition of claim 1, wherein the polynucleotide comprises RNA, and optionally wherein said RNA is partially or fully human codon optimized.

3. The composition of claims 1 or 2, wherein the polynucleotide further comprises or encodes: a 5′ untranslated region (UTR), a 5′ cap, a 3′ UTR, a 3′ tailing sequence, or any combination thereof.

4. The composition of claim 3, wherein the 5′ UTR, 3′ UTR, or both are heterologous to the polynucleotide encoding at least a portion of SCF1.

5. The composition of claims 3 or 4, wherein the 3′ tailing sequence comprises a polyA tail, a polyG quartet, a stem loop sequence, a triple helix forming sequence, a tRNA-like sequence, or any combination thereof.

6. The composition of any of claims 1-5, wherein the polynucleotide comprises at least one chemically modified nucleotide.

7. The composition of claim 6, wherein the at least one chemically modified nucleotide comprises a modified uracil.

8. The composition of claim 7, wherein at least 60% of the uracil in the polynucleotide encoding at least a portion of SCF1 are chemically modified.

9. The composition of any of claims 6-8, wherein the at least one chemically modified nucleotide comprises 5-methylcytosine or N1-methylpseudouridine (m1Ψ).

10. The composition of any of claims 1-9, wherein the polynucleotide comprises a nucleotide sequence having: i) at least 75% sequence identity to SEQ ID NOs: 2, 3, 15, 17, or 18, or a complement or reverse complement thereof, wherein T may be replaced by U, and/or ii) at least 50 consecutive nucleotides from SEQ ID NOs: 2, 3, 15, 17, or 18, or a complement or reverse complement thereof, wherein T may be replaced by U.

11. The composition of any of claims 1-10, wherein the SCF1 comprises: i) an amino acid sequence having at least 90% identity to SEQ ID NO:1, 4, or 16 and/or ii) at least 12, at least 15, or at least 20, consecutive amino acids from SEQ ID NO:1, 4 or 16.

12. The composition of any of claims 1-9, wherein SCF1 comprises: i) an amino acid sequence having at least 90% identity to any one of SEQ ID NOs: 5-14 or 16, and/or ii) at least 12 or 15 consecutive amino acids from any one of SEQ ID NOs: 5-14 or 16.

13. A messenger ribonucleic acid (mRNA) comprising:
i) a 5′ untranslated region (UTR);
ii) an open reading frame encoding at least a portion of Surface Colonization Factor (SCF1) from Candida auris; and
iii) a 3′ UTR,
wherein the mRNA is at least partially optimized for enhanced expression, productive co-translational protein folding, increased stability, or a combination thereof.

14. The mRNA of claim 13, wherein the 5′ UTR, 3′ UTR, or both are heterologous to the open reading frame encoding at least a portion of SCF1, and/or wherein the RNA is partially or fully human codon optimized.

15. The mRNA of claim 13 or 14, wherein the mRNA further comprises or encodes: a 5′ cap, a 3′ tailing sequence, or both.

16. The mRNA of claim 15, wherein the 3′ tailing sequence comprises a polyA tail, a polyG quartet, a stem loop sequence, a triple helix forming sequence, a tRNA-like sequence, or any combination thereof.

17. The mRNA of any of claims 13-16, wherein the mRNA comprises at least one chemically modified nucleotide.

18. The mRNA of claim 17, wherein the at least one chemically modified nucleotide comprises a modified uracil.

19. The mRNA of claim 18, wherein at least 60% of the uracil in the open reading frame encoding at least a portion of SCF1 are chemically modified.

20. The mRNA of any of claims 17-19, wherein the at least one chemically modified nucleotide comprises 5-methylcytosine or N1-methylpseudouridine (m1Ψ).

21. The mRNA of any of claims 13-20, wherein the mRNA comprises a nucleotide sequence having at least 70% sequence identity to SEQ ID NO:3, 15, 17, 18, and/or comprises at least 15, or at least 25, consecutive nucleotides from SEQ ID NO:3, 15, 17, or 18.

22. The mRNA of any of claims 13-20, wherein SCF1 comprises: i) an amino acid sequence having at least 90% identity to SEQ ID NOs:1, 4-14 or 16, and/or ii) at least 12 or 15 consecutive amino acids from SEQ ID NOs:1-14 or 16.

23. A vaccine comprising:
the composition of any of claims 1-12 or the mRNA of any of claims 13-22; and
at least one adjuvant, a delivery vehicle, or a combination thereof.

24. The vaccine of claim 23, wherein the delivery vehicle comprises a lipid nanoparticle encapsulating the composition or the mRNA.

25. The vaccine of claim 24, wherein the lipid nanoparticle comprises a cationic lipid, a neutral and/or non-cationic lipid, a sterol, or any combination thereof.

26. The vaccine of claim 25, wherein the non-cationic lipid comprises a phospholipid.

27. The vaccine of claim 25 or 26, wherein the sterol comprises cholesterol or a modification or ester thereof.

28. The vaccine of any of claims 24-27, wherein the lipid nanoparticle comprises a polyethylene glycol (PEG)-lipid conjugate.

29. A composition comprising: an immunogenic protein comprising at least a portion of Surface Colonization Factor (SCF1) from Candida auris,
wherein said immunogenic protein is conjugated to a hapten, and/or wherein said composition further comprises an adjuvant and/or a delivery vehicle.

30. The composition of claim 29, wherein said immunogenic protein comprises: i) an amino acid sequence having at least 90% identity to SEQ ID NO:1, 4, or 16 and/or ii) at least 10 or at least 12 or at least 15 or at least 20 consecutive amino acids from SEQ ID NO:1, 4 or 16.

31. The composition of claim 29, wherein SCF1 comprises: i) an amino acid sequence having at least 90% identity to any one of SEQ ID NOs: 5-14 or 16, and/or ii) at least 10, 12, or 15 or at least 20 consecutive amino acids from any one of SEQ ID NOs: 5-14 or 16.

32. The composition of any of claims 29-31, wherein said hapten is selected from the group consisting of: fluorescein, biotin, digoxigenin, and dinitrophenol.

33. A method of treating or preventing skin infection and/or systemic infection by Candida auris comprising: administering the composition of any of claims 1-12 or 29-32, the mRNA of any of claims 13-22, or the vaccine of any of claims 23-28, to a subject in need thereof.

34. The method of claim 33, wherein said subject has said skin infection.

35. The method of claim 33, wherein said subject has said systemic infection.

36. The method of any of claims 33-36, wherein the administering comprises an initial immunization and at least one subsequent immunization.

37. A method of inducing an immune response in a subject comprising: administrating the composition of any of claims 1-12 or 29-32, the mRNA of any of claims 13-22, or the vaccine of any of claims 23-28 to a subject in need thereof.

38. The method of any of claims 33-37, wherein the subject is human.

39. Use of the composition of any of claims 1-12 or 29-32, or the mRNA of any of claims 13-22, in the manufacture of a medicament for the treatment or prevention skin infection and/or systemic infection by Candida auris.

40. A method comprising: treating a subject with a composition comprising a first agent,
wherein said subject has a skin infection and/or systemic infection by Candida auris, and
wherein said first agent is selected from the following:
a) an anti-SCF1 antibody or antigen binding fragment thereof,
b) an antisense sequence specific for SCF1 DNA or mRNA,
c) a siRNA sequence specific for SCF1 mRNA;
d) an antisense sequence specific for C. auris Swi1, BCY1, EFG1, IRF1, and TPK2, DNA or mRNA,
e) a siRNA sequence specific for C. auris Swi1, BCY1, EFG1, IRF1, and TPK2 mRNA; and
f) a small molecule that inhibits the function of one of the following proteins of C. auris: SCF1, BCY1, EFG1, IRF1, and TPK2.

41. The method of claim 40, wherein said subject has said skin infection.

42. The method of claim 41, wherein said treating comprises applying said composition topically to said skin infection of said subject.

43. The method of claim 40, wherein said treating comprises providing said composition to said subject such that said subject is able to apply said composition to said skin infection.

44. The method of claim 42 or 43, wherein said applying said composition, or providing said composition, is repeated daily for at least 7 days, or for at least 14 days, or for at least 21 days.

45. The method of claim 40, wherein said composition further comprises a hydrogel agent, and said composition is in the form of a hydrogel.

46. The method of claim 40, wherein said composition further comprises a solubilizer and/or emulsifier.

47. The method of claim 40, wherein said composition is in the form of a cream, emulsion, skin lotion, gel, an ointment, a spray, or is present in a skin patch.

48. The method of claim 40, wherein said subject is a human.

49. The method of claim 40, wherein said subject has a systemic infection with C. auris.

50. The method of claim 40, wherein said treating comprises intravenous injection of said composition.

51. A method comprising: treating a surface of an object with a composition comprising a first agent,
wherein said surface of said object is suspected of harboring Candida auris, and
wherein said first agent is selected from the following:
a) an anti-SCF1 antibody or antigen binding fragment thereof,
b) an antisense sequence specific for SCF1 DNA or mRNA,
c) a siRNA sequence specific for SCF1 mRNA;
d) an antisense sequence specific for C. auris Swi1, BCY1, EFG1, IRF1, and TPK2, DNA or mRNA,
e) a siRNA sequence specific for C. auris Swi1, BCY1, EFG1, IRF1, and TPK2 mRNA; and
f) a small molecule that inhibits the function of one of the following proteins of C. auris: SCF1, BCY1, EFG1, IRF1, and TPK2.

52. The method of claim 51, wherein said composition is in the form of a foam or liquid, and wherein said treating comprises spraying said composition on said surface of said object.

53. The method of claim 51, wherein said composition further comprises a solubilizer and/or emulsifier.

54. The method of claim 51, wherein said object comprises medical equipment or a surgical bed.

55. The method of claim 54, wherein said medical equipment is selected from: a surgical tool, catheter, a kidney tray, surgical scissors, and surgical clamps.


SEQUENCE LISTING (206 sequences)

SEQ ID NO: 1 - protein, 765 aa, Candida auris
MRFSLLLAGVVAADSGYLMARHLHNKPIGTTTLPADDDYCTPSKHYWVKGGHHRRSNDGPEFIDAGVAVSDVTKVGDNVYELSLNFKTAEDEELAKAISNNQVRSLTIEGTGVEDVQLIGRDNKMEGIPWTKWTARVRVRSERMLEKRHKHKPAGVVCCLPHKLEVRLALEPEGEAARAYKSVFSRDSYAYTVERGVGDEITEVGDVDGFLNNVLKRDVDSPLSKRLGVFNRVRNLQPRFHKQCWVCECETTTTTSLSSSSSSTVPPTTTSESSTSETTSSLSSKTEPSGSSSLNPPPATETSSETTSSTETSSSETTSTETSTEITSSSEATSSTETSTETTSSVETTSSTETTSSSETSNTETTSTETTSSTETTSSSETSTEPTTSSESTTSPGPATSSEQTTSSEPTTSTNTSSETKPTESETGSSTESSSSTNVTTETTPTESTESTTSGPGSSTEPPSVTTSKTTSEQTDSHTGPSTSNTSTGTETSTESQTTPTDGHTGPHTEPTTVTITGSTSTAIVTYTPTQPGPEHPQPSGPSGPQGPEGPEGPEGPEGPEGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPSGPGENGGDVEQPKGEQPEGNGGKGGPGGPSGPGGPGGHGSEGNHGGPAPAPSVSQVGQPGPSASVSQYTGGAVRLATTPLGALLAAALLL

SEQ ID NO: 2 - DNA, 2298 bp, Candida auris
  source 1..2298: mol_type = genomic DNA
atgagattctctttacttcttgcaggtgtagtggctgctgatagtgggtacttgatggcaaggcacttacacaacaagcccatcggcactaccaccttgccagctgacgacgactactgtacaccttctaagcactactgggtgaaaggtggccatcatcgtcgttccaacgatggccccgagtttatcgatgcgggagtggcagtcagtgatgtcactaaggtcggtgacaacgtctacgagttgtccttgaatttcaagaccgccgaggatgaggaattagcaaaggccatttccaacaaccaggtgcgtagcttgactatcgaaggcactggagtcgaagatgtgcagttgattggcagagacaacaaaatggagggaattccatggactaagtggacggccagagtgagagtgaggtcggaacggatgctcgagaagagacacaagcacaaaccagccggcgttgtgtgctgtctcccacataagttggaggttcgtcttgctttggagccagaaggagaagctgctcgtgcctacaaatctgttttttccagggactcctatgcctacactgttgaaagaggtgttggagatgaaatcaccgaagtgggagatgtggatgggttcctcaataacgtcttgaagagagatgtggactctcctctctccaaaagactgggagtgttcaacagggtgagaaacttacaaccaaggttccacaagcagtgttgggtgtgtgagtgtgaaacaaccaccactacttcgctgagctcgtcttcttcttctactgttccacctaccaccacaagcgagagctcaacctcggagactaccagctcactgtcgtccaaaacagagccttctggcagttcgtcactgaatcctcctcctgcaactgagacatcttcagagaccacttccagtactgagacttcgagctctgagactacttccactgagaccagcactgagattacttccagctctgaggcaacctcgtctactgagaccagtactgagactacctctagtgttgagactacttccagtactgagactacctccagttcagagacctccaatactgagactacctctactgagacaacctcaagtactgagaccacttcaagctctgaaaccagtactgagccaactacttcatcagagtcaactacctcacctgggccagctacttcatctgagcaaactacctcatctgagccaactacctccacaaacacttcctcagagacgaagcccactgaatccgaaactggatccagtactgagtcaagctcttccacaaatgtgaccaccgaaacaacgccaaccgaatcaactgagtcaaccacctcagggccaggctccagcactgaaccaccttctgttactacttcgaagacgacttctgagcaaactgactctcacactggtccatccacttcaaacacgagtactggcacagaaacaagtactgagtctcagaccacgccaactgacggccacactggccctcacactgagcccacaactgtgacaattaccggttcaactagtacagctattgtgacttatacgccaactcagccaggccctgaacacccacaaccaagtgggcctagtggaccacaaggaccagagggaccagagggaccagagggaccagagggacctgaaggaccaagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggtccaagcggcccaagtggtccaagcggtccaagcggtccaagcggtccaagcggtccaagtggtccaagcggtccaagcggtcccggtgaaaacggtggagacgttgaacagccaaagggtgaacagccagaaggtaacggtggcaaaggtggtccaggcggcccaagtggtccaggaggtccagggggccatggcagtgaaggcaaccacgggggacctgctcctgctccttctgtctcccaagttggccagccaggaccctctgctagtgtatctcagtacactgggggagctgtgagacttgcaaccacgccgcttggcgctttgcttgctgctgccttgttgctttaa

SEQ ID NO: 3 - RNA, 2298 nt, Candida auris
  source 1..2298: mol_type = genomic RNA
atgagattctctttacttcttgcaggtgtagtggctgctgatagtgggtacttgatggcaaggcacttacacaacaagcccatcggcactaccaccttgccagctgacgacgactactgtacaccttctaagcactactgggtgaaaggtggccatcatcgtcgttccaacgatggccccgagtttatcgatgcgggagtggcagtcagtgatgtcactaaggtcggtgacaacgtctacgagttgtccttgaatttcaagaccgccgaggatgaggaattagcaaaggccatttccaacaaccaggtgcgtagcttgactatcgaaggcactggagtcgaagatgtgcagttgattggcagagacaacaaaatggagggaattccatggactaagtggacggccagagtgagagtgaggtcggaacggatgctcgagaagagacacaagcacaaaccagccggcgttgtgtgctgtctcccacataagttggaggttcgtcttgctttggagccagaaggagaagctgctcgtgcctacaaatctgttttttccagggactcctatgcctacactgttgaaagaggtgttggagatgaaatcaccgaagtgggagatgtggatgggttcctcaataacgtcttgaagagagatgtggactctcctctctccaaaagactgggagtgttcaacagggtgagaaacttacaaccaaggttccacaagcagtgttgggtgtgtgagtgtgaaacaaccaccactacttcgctgagctcgtcttcttcttctactgttccacctaccaccacaagcgagagctcaacctcggagactaccagctcactgtcgtccaaaacagagccttctggcagttcgtcactgaatcctcctcctgcaactgagacatcttcagagaccacttccagtactgagacttcgagctctgagactacttccactgagaccagcactgagattacttccagctctgaggcaacctcgtctactgagaccagtactgagactacctctagtgttgagactacttccagtactgagactacctccagttcagagacctccaatactgagactacctctactgagacaacctcaagtactgagaccacttcaagctctgaaaccagtactgagccaactacttcatcagagtcaactacctcacctgggccagctacttcatctgagcaaactacctcatctgagccaactacctccacaaacacttcctcagagacgaagcccactgaatccgaaactggatccagtactgagtcaagctcttccacaaatgtgaccaccgaaacaacgccaaccgaatcaactgagtcaaccacctcagggccaggctccagcactgaaccaccttctgttactacttcgaagacgacttctgagcaaactgactctcacactggtccatccacttcaaacacgagtactggcacagaaacaagtactgagtctcagaccacgccaactgacggccacactggccctcacactgagcccacaactgtgacaattaccggttcaactagtacagctattgtgacttatacgccaactcagccaggccctgaacacccacaaccaagtgggcctagtggaccacaaggaccagagggaccagagggaccagagggaccagagggacctgaaggaccaagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggacctagtggtccaagcggcccaagtggtccaagcggtccaagcggtccaagcggtccaagcggtccaagtggtccaagcggtccaagcggtcccggtgaaaacggtggagacgttgaacagccaaagggtgaacagccagaaggtaacggtggcaaaggtggtccaggcggcccaagtggtccaggaggtccagggggccatggcagtgaaggcaaccacgggggacctgctcctgctccttctgtctcccaagttggccagccaggaccctctgctagtgtatctcagtacactgggggagctgtgagacttgcaaccacgccgcttggcgctttgcttgctgctgccttgttgctttaa

SEQ ID NO: 4 - protein, 227 aa, Candida auris
IGTTTLPADDDYCTPSKHYWVKGGHHRRSNDGPEFIDAGVAVSDVTKVGDNVYELSLNFKTAEDEELAKAISNNQVRSLTIEGTGVEDVQLIGRDNKMEGIPWTKWTARVRVRSERMLEKRHKHKPAGVVCCLPHKLEVRLALEPEGEAARAYKSVFSRDSYAYTVERGVGDEITEVGDVDGFLNNVLKRDVDSPLSKRLGVFNRVRNLQPRFHKQCWVCECETTTT

SEQ ID NO: 5 - protein, 33 aa, Candida auris
IGTTTLPADDDYCTPSKHYWVKGGHHRRSNDGP

SEQ ID NO: 6 - protein, 43 aa, Candida auris
GRDNKMEGIPWTKWTARVRVRSERMLEKRHKHKPAGVVCCLPH

SEQ ID NO: 7 - protein, 85 aa, Candida auris
EFIDAGVAVSDVTKVGDNVYELSLNFKTAEDEELAKAISNNQVRSLTIEGTGVEDVQLIGRDNKMEGIPWTKWTARVRVRSERML

SEQ ID NO: 8 - protein, 55 aa, Candida auris
KLEVRLALEPEGEAARAYKSVFSRDSYAYTVERGVGDEITEVGDVDGFLNNVLKR

SEQ ID NO: 9 - protein, 28 aa, Candida auris
LPADDDYCTPSKHYWVKGGHHRRSNDGP

SEQ ID NO: 10 - protein, 28 aa, Candida auris
IGTTTLPADDDYCTPSKHYWVKGGHHRR

SEQ ID NO: 11 - protein, 23 aa, Candida auris
LPADDDYCTPSKHYWVKGGHHRR

SEQ ID NO: 12 - protein, 20 aa, Candida auris
DDDYCTPSKHYWVKGGHHRR

SEQ ID NO: 13 - protein, 20 aa, Candida auris
LPADDDYCTPSKHYWVKGGH

SEQ ID NO: 14 - protein, 17 aa, Candida auris
DDDYCTPSKHYWVKGGH

SEQ ID NO: 15 - DNA, 2298 bp, Candida auris; identical to SEQ ID NO: 2
  source 1..2298: mol_type = genomic DNA

SEQ ID NO: 16 - protein, 13 aa, Candida auris
GGHHRRSNDGPEF

SEQ ID NO: 17 - DNA, 39 bp, Candida auris
  source 1..39: mol_type = genomic DNA
ggtggccatcatcgtcgttccaacgatggccccgagttt

SEQ ID NO: 18 - RNA, 39 nt, Candida auris
  source 1..39: mol_type = genomic RNA
ggtggccatcatcgtcgttccaacgatggccccgagttt

SEQ ID NO: 19 - protein, 11 aa, Candida auris
GHHRRSNDGPE

SEQ ID NO: 20 - protein, 177 aa, Saccharomyces cerevisiae
SSEGTSCNSIVNGCPNLDFNWHMDQQNIMQYTLDVTSVSWVQDNTYQITIHVKGKENIDLKYLWSLKIIGVTGPKGTVQLYGYNENTYLIDNPTDFTATFEVYATQDVNSCQVWMPNFQIQFEYLQGSAAQYASSWQWGTTSFDLSTGCNNYDNQGHSQTDFPGFYWNIDCDNNCGG

SEQ ID NO: 21 - protein, 227 aa, Candida auris
IGTTTLPADDDYCTPSKHYWVKGGHHRRSNDGPEFIDAGVAVSDVTKVGDNVYELSLNFKTAEDEELAKAISNNQVRSLTIEGTGVEDVQLIGRDNKMEGIPWTKWTARVRVRSERMLEKRHKHKPAGVVCCLPHKLEVRLALEPEGEAARAYKSVFSRDSYAYTVERGVGDEITEVGDVDGFLNNVLKRDVDSPLSKRSGVFNRVRNLQPRFHKQCWVCECETTTT

SEQ ID NO: 22 - DNA, 20 bp, Synthetic construct
  source 1..20: mol_type = other DNA
cgtgctgtgttcccatccat

SEQ ID NO: 23 - DNA, 20 bp, Synthetic construct
  source 1..20: mol_type = other DNA
agcctcatcaccgacatacg

SEQ ID NO: 24 - DNA, 20 bp, Synthetic construct
  source 1..20: mol_type = other DNA
gtgagagtgaggtcggaacg

SEQ ID NO: 25 - DNA, 20 bp, Synthetic construct
  source 1..20: mol_type = other DNA
cagcttctccttctggctcc

SEQ ID NO: 26 - DNA, 20 bp, Synthetic construct
  source 1..20: mol_type = other DNA
gggagacaccttgacgcttt

SEQ ID NO: 27 - DNA, 20 bp, Synthetic construct
  source 1..20: mol_type = other DNA
gttggctcagggaagtcgaa

SEQ ID NO: 28 - DNA, 20 bp, Synthetic construct
  source 1..20: mol_type = other DNA
tgagagatttagagccgccg

SEQ ID NO: 29 - DNA, 20 bp, Synthetic construct
  source 1..20: mol_type = other DNA
tacgctctgcaatacctggg

SEQ ID NO: 30 - DNA, 20 bp, Synthetic construct
  source 1..20: mol_type = other DNA
ttggtgaaggagcaaccgag

SEQ ID NO: 31 - DNA, 20 bp, Synthetic construct
  source 1..20: mol_type = other DNA
ggggcttcaagtgtctgact

SEQ ID NO: 32 - DNA, 23 bp, Synthetic construct
  source 1..23: mol_type = other DNA
cctctttgtagttcaacttatgc

SEQ ID NO: 33 - DNA, 20 bp, Synthetic construct
  source 1..20: mol_type = other DNA
gtcccaaaaccttctcaagc

SEQ ID NO: 34 - DNA, 17 bp, Synthetic construct
  source 1..17: mol_type = other DNA
caggaaacagctatgac

SEQ ID NO: 35 - DNA, 16 bp, Synthetic construct
  source 1..16: mol_type = other DNA
gtaaaacgacggccag

SEQ ID NO: 36 - DNA, 42 bp, Synthetic construct
  source 1..42: mol_type = other DNA
gactacaaggacgacgatgacaagacaaccaccactacttcg

SEQ ID NO: 37 - DNA, 44 bp, Synthetic construct
  source 1..44: mol_type = other DNA
cttgtcatcgtcgtccttgtagtcttcacactcacacacccaac

SEQ ID NO: 38 - DNA, 17 bp, Synthetic construct
  source 1..17: mol_type = other DNA
gctattacgccagctgg

SEQ ID NO: 39 - DNA, 20 bp, Synthetic construct
  source 1..20: mol_type = other DNA
cgcaattaatgtgagttagc

SEQ ID NO: 40 - DNA, 17 bp, Synthetic construct
  source 1..17: mol_type = other DNA
cagtggagcggaaactc

SEQ ID NO: 41 - DNA, 19 bp, Synthetic construct
  source 1..19: mol_type = other DNA
gtggacctgatttgactgg

SEQ ID NO: 42 - DNA, 19 bp, Synthetic construct
  source 1..19: mol_type = other DNA
cctgaatctctcaacgaag

SEQ ID NO: 43 - DNA, 18 bp, Synthetic construct
  source 1..18: mol_type = other DNA
gtggtgtaagcgattgtg

SEQ ID NO: 44 - DNA, 18 bp, Synthetic construct
  source 1..18: mol_type = other DNA
gcaagacgaacctccaac

SEQ ID NO: 45 - DNA, 18 bp, Synthetic construct
  source 1..18: mol_type = other DNA
ggatcctctagagtcgac

SEQ ID NO: 46 - DNA, 18 bp, Synthetic construct
  source 1..18: mol_type = other DNA
ggtaccgagctcgaattc

SEQ ID NO: 47 - DNA, 18 bp, Synthetic construct
  source 1..18: mol_type = other DNA
actggatggcggcgttag

SEQ ID NO: 48 - DNA, 21 bp, Synthetic construct
  source 1..21: mol_type = other DNA
cgacatggaggcccagaatac

SEQ ID NO: 49 - DNA, 18 bp, Synthetic construct
  source 1..18: mol_type = other DNA
atcaagcttgcctcgtcc

SEQ ID NO: 50 - DNA, 38 bp, Synthetic construct
  source 1..38: mol_type = other DNA
ttcgagctcggtaccgtactcatcagagtatacgatgc

SEQ ID NO: 51 - DNA, 41 bp, Synthetic construct
  source 1..41: mol_type = other DNA
acgccgccatccagtgagcagtcgatgcttcaaattaaatc

SEQ ID NO: 52 - DNA, 33 bp, Synthetic construct
  source 1..33: mol_type = other DNA
aagcatcgactgctcactggatggcggcgttag

SEQ ID NO: 53 - DNA, 33 bp, Synthetic construct
  source 1..33: mol_type = other DNA
gagaacctaaaaattatcaagcttgcctcgtcc

SEQ ID NO: 54 - DNA, 35 bp, Synthetic construct
  source 1..35: mol_type = other DNA
cgaggcaagcttgataatttttaggttctctagcc

SEQ ID NO: 55 - DNA, 35 bp, Synthetic construct
  source 1..35: mol_type = other DNA
gactctagaggatcccggagtgtcacatttctttc

SEQ ID NO: 56 - DNA, 33 bp, Synthetic construct
  source 1..33: mol_type = other DNA
aaatgtgacactccgggatcctctagagtcgac

SEQ ID NO: 57 - DNA, 33 bp, Synthetic construct
  source 1..33: mol_type = other DNA
tactctgatgagtacggtaccgagctcgaattc

SEQ ID NO: 58 - DNA, 40 bp, Synthetic construct
  source 1..40: mol_type = other DNA
tagccttgatttgagcaaccggatcctctagagtcgacct

SEQ ID NO: 59 - DNA, 40 bp, Synthetic construct
  source 1..40: mol_type = other DNA
tacaaatagcagttatcagcggtaccgagctcgaattcac

SEQ ID NO: 60 - DNA, 44 bp, Synthetic construct
  source 1..44: mol_type = other DNA
gtgaattcgagctcggtaccgctgataactgctatttgtataca

SEQ ID NO: 61 - DNA, 45 bp, Synthetic construct
  source 1..45: mol_type = other DNA
tactaacgccgccatccagttgataaatggaaagtagagaaacat

SEQ ID NO: 62 - DNA, 40 bp, Synthetic construct
  source 1..40: mol_type = other DNA
tctctactttccatttatcaactggatggcggcgttagta

SEQ ID NO: 63 - DNA, 38 bp, Synthetic construct
  source 1..38: mol_type = other DNA
aaataataattgagtaggccatcaagcttgcctcgtcc

SEQ ID NO: 64 - DNA, 47 bp, Synthetic construct
  source 1..47: mol_type = other DNA
ggacgaggcaagcttgatggcctactcaattattatttaattttttt

SEQ ID NO: 65 - DNA, 40 bp, Synthetic construct
  source 1..40: mol_type = other DNA
aggtcgactctagaggatccggttgctcaaatcaaggcta

SEQ ID NO: 66 - DNA, 40 bp, Synthetic construct
  source 1..40: mol_type = other DNA
gactatcctgaaaacgcttgggatcctctagagtcgacct

SEQ ID NO: 67 - DNA, 40 bp, Synthetic construct
  source 1..40: mol_type = other DNA
gcgtttacggcgattgcaccggtaccgagctcgaattcac

SEQ ID NO: 68 - DNA, 40 bp, Synthetic construct
  source 1..40: mol_type = other DNA
gtgaattcgagctcggtaccggtgcaatcgccgtaaacgc

SEQ ID NO: 69 - DNA, 44 bp, Synthetic construct
  source 1..44: mol_type = other DNA
tactaacgccgccatccagtgaaagatgatgggaaacaaggtga

SEQ ID NO: 70 - DNA, 40 bp, Synthetic construct
  source 1..40: mol_type = other DNA
cttgtttcccatcatctttcactggatggcggcgttagta

SEQ ID NO: 71 - DNA, 38 bp, Synthetic construct
  source 1..38: mol_type = other DNA
ggtatcataaaagctcacgtatcaagcttgcctcgtcc

SEQ ID NO: 72 - DNA, 39 bp, Synthetic construct
  source 1..39: mol_type = other DNA
ggacgaggcaagcttgatacgtgagcttttatgatacct

SEQ ID NO: 73 - DNA, 40 bp, Synthetic construct
  source 1..40: mol_type = other DNA
aggtcgactctagaggatcccaagcgttttcaggatagtc

SEQ ID NO: 74 - DNA, 33 bp, Synthetic construct
  source 1..33: mol_type = other DNA
ttcgagctcggtaccccttctcgagttactctg

SEQ ID NO: 75 - DNA, 36 bp, Synthetic construct
  source 1..36: mol_type = other DNA
acgccgccatccagttggataaagcaagtgaaaaag

SEQ ID NO: 76 - DNA, 33 bp, Synthetic construct
  source 1..33: mol_type = other DNA
cacttgctttatccaactggatggcggcgttag

SEQ ID NO: 77 - DNA, 33 bp, Synthetic construct
  source 1..33: mol_type = other DNA
agtgggtatatggtcatcaagcttgcctcgtcc

SEQ ID NO: 78 - DNA, 33 bp, Synthetic construct
  source 1..33: mol_type = other DNA
cgaggcaagcttgatgaccatatacccactcgc

SEQ ID NO: 79 - DNA, 34 bp, Synthetic construct
  source 1..34: mol_type = other DNA
gactctagaggatcccgccactttacttgccaac

SEQ ID NO: 80 - DNA, 33 bp, Synthetic construct
  source 1..33: mol_type = other DNA
gcaagtaaagtggcgggatcctctagagtcgac

SEQ ID NO: 81 - DNA, 33 bp, Synthetic construct
  source 1..33: mol_type = other DNA
agtaactcgagaaggggtaccgagctcgaattc

SEQ ID NO: 82 - DNA, 34 bp, Synthetic construct
  source 1..34: mol_type = other DNA
ttcgagctcggtacccagacatcttttgtgcaag

SEQ ID NO: 83 - DNA, 33 bp, Synthetic construct
  source 1..33: mol_type = other DNA
acgccgccatccagtttgttgatggggaactac

SEQ ID NO: 84 - DNA, 33 bp, Synthetic construct
  source 1..33: mol_type = other DNA
gttccccatcaacaaactggatggcggcgttag

SEQ ID NO: 85 - DNA, 33 bp, Synthetic construct
  source 1..33: mol_type = other DNA
aaagtgacaccgtcaatcaagcttgcctcgtcc

SEQ ID NO: 86 - DNA, 34 bp, Synthetic construct
  source 1..34: mol_type = other DNA
cgaggcaagcttgattgacggtgtcactttcgag

SEQ ID NO: 87 - DNA, 33 bp, Synthetic construct
  source 1..33: mol_type = other DNA
gactctagaggatccgtggtgtaagcgattgtg

SEQ ID NO: 88 - DNA, 33 bp, Synthetic construct
  source 1..33: mol_type = other DNA
aatcgcttacaccacggatcctctagagtcgac

SEQ ID NO: 89 - DNA, 33 bp, Synthetic construct
  source 1..33: mol_type = other DNA
cacaaaagatgtctgggtaccgagctcgaattc

SEQ ID NO: 90 - DNA, 51 bp, Synthetic construct
  source 1..51: mol_type = other DNA
acgacggccagtgaattcgagctcggtacccgtattctcgagctcttaaag

SEQ ID NO: 91 - DNA, 49 bp, Synthetic construct
  source 1..49: mol_type = other DNA
acgccgccatccagtaatggttgattaacaaaagaaaaaaaaaaaaaag

SEQ ID NO: 92 - DNA, 33 bp, Synthetic construct
  source 1..33: mol_type = other DNA
tgttaatcaaccattactggatggcggcgttag

SEQ ID NO: 93 - DNA, 33 bp, Synthetic construct
  source 1..33: mol_type = other DNA
gctaggaaaaattgcatcaagcttgcctcgtcc

SEQ ID NO: 94 - DNA, 42 bp, Synthetic construct
  source 1..42: mol_type = other DNA
cgaggcaagcttgatgcaatttttcctagcctttattttttc

SEQ ID NO: 95 - DNA, 48 bp, Synthetic construct
  source 1..48: mol_type = other DNA
gcatgcctgcaggtcgactctagaggatccctaccaaaagttgacgtg

SEQ ID NO: 96 - DNA, 43 bp, Synthetic construct
  source 1..43: mol_type = other DNA
ggccagtgaattcgagctcggtaccgcaaacgtcagtctaccg

SEQ ID NO: 97 - DNA, 43 bp, Synthetic construct
  source 1..43: mol_type = other DNA
ttcgatactaacgccgccatccagtgatggaatggatggagag

SEQ ID NO: 98 - DNA, 51 bp, Synthetic construct
  source 1..51: mol_type = other DNA
gagggtattctgggcctccatgtcgctttaaaatcgttttatttagttctg

SEQ ID NO: 99 - DNA, 51 bp, Synthetic construct
  source 1..51: mol_type = other DNA
cctgcaggtcgactctagaggatccgtacagcttacacaactaataaaaac

SEQ ID NO: 100 - DNA, 33 bp, Synthetic construct
  source 1..33: mol_type = other DNA
ttcgagctcggtaccggtcttgttaacatggcc

SEQ ID NO: 101 - DNA, 33 bp, Synthetic construct
  source 1..33: mol_type = other DNA
acgccgccatccagtgatgctcaatagctgaag

SEQ ID NO: 102 - DNA, 33 bp, Synthetic construct
  source 1..33: mol_type = other DNA
cagctattgagcatcactggatggcggcgttag

SEQ ID NO: 103 - DNA, 33 bp, Synthetic construct
  source 1..33: mol_type = other DNA
aattggtacgttaggatcaagcttgcctcgtcc

SEQ ID NO: 104 - DNA, 33 bp, Synthetic construct
  source 1..33: mol_type = other DNA
cgaggcaagcttgatcctaacgtaccaattctc

SEQ ID NO: 105 - DNA, 36 bp, Synthetic construct
  source 1..36: mol_type = other DNA
gactctagaggatccccaaaaatatgaagacgagag

SEQ ID NO: 106 - DNA, 33 bp, Synthetic construct
  source 1..33: mol_type = other DNA
tcttcatatttttggggatcctctagagtcgac

SEQ ID NO: 107 - DNA, 33 bp, Synthetic construct
  source 1..33: mol_type = other DNA
catgttaacaagaccggtaccgagctcgaattc

SEQ ID NO: 108 - DNA, 36 bp, Synthetic construct
  source 1..36: mol_type = other DNA
ttcgagctcggtaccgcagctgcattggtacatatg

SEQ ID NO: 109 - DNA, 34 bp, Synthetic construct
  source 1..34: mol_type = other DNA
acgccgccatccagtgtggagcaggttgggttag

SEQ ID NO: 110 - DNA, 33 bp, Synthetic construct
  source 1..33: mol_type = other DNA
cccaacctgctccacactggatggcggcgttag

SEQ ID NO: 111 - DNA, 33 bp, Synthetic construct
  source 1..33: mol_type = other DNA
tttgtgcatttaagtatcaagcttgcctcgtcc

SEQ ID NO: 112 - DNA, 34 bp, Synthetic construct
  source 1..34: mol_type = other DNA
cgaggcaagcttgatacttaaatgcacaaaacgc

SEQ ID NO: 113 - DNA, 33 bp, Synthetic construct
  source 1..33: mol_type = other DNA
gactctagaggatccggactacagcttctttgg

SEQ ID NO: 114 - DNA, 33 bp, Synthetic construct
  source 1..33: mol_type = other DNA
aagaagctgtagtccggatcctctagagtcgac

SEQ ID NO: 115 - DNA, 33 bp, Synthetic construct
  source 1..33: mol_type = other DNA
taccaatgcagctgcggtaccgagctcgaattc

SEQ ID NO: 116 - DNA, 59 bp, Synthetic construct
  source 1..59: mol_type = other DNA
acgttgtaaaacgacggccagtgaattcgagctcggtacccaaagtagccacacacttg

SEQ ID NO: 117 - DNA, 40 bp, Synthetic construct
  source 1..40: mol_type = other DNA
tactaacgccgccatccagtatttggcagagagtagaatg

SEQ ID NO: 118 - DNA, 38 bp, Synthetic construct
  source 1..38: mol_type = other DNA
cattctactctctgccaaatactggatggcggcgttag

SEQ ID NO: 119 - DNA, 38 bp, Synthetic construct
  source 1..38: mol_type = other DNA
tgtgcagccttgtatcaacaatcaagcttgcctcgtcc

SEQ ID NO: 120 - DNA, 39 bp, Synthetic construct
  source 1..39: mol_type = other DNA
ggggacgaggcaagcttgattgttgatacaaggctgcac

SEQ ID NO: 121 - DNA, 58 bp, Synthetic construct
  source 1..58: mol_type = other DNA
cgccaagcttgcatgcctgcaggtcgactctagaggatcccttcgtcctcgtgcttgg

SEQ ID NO: 122 - DNA, 58 bp, Synthetic construct
  source 1..58: mol_type = other DNA
acgttgtaaaacgacggccagtgaattcgagctcggtaccgaacgcaacaacttaagc

SEQ ID NO: 123 - DNA, 39 bp, Synthetic construct
  source 1..39: mol_type = other DNA
tactaacgccgccatccagtgcgtttgtgtttgatggag

SEQ ID NO: 124 - DNA, 38 bp, Synthetic construct
  source 1..38: mol_type = other DNA
tctccatcaaacacaaacgcactggatggcggcgttag

SEQ ID NO: 125 - DNA, 38 bp, Synthetic construct
  source 1..38: mol_type = other DNA
ataaaggtttcgtcttttgtatcaagcttgcctcgtcc

SEQ ID NO: 126 - DNA, 45 bp, Synthetic construct
  source 1..45: mol_type = other DNA
ggggacgaggcaagcttgatacaaaagacgaaacctttatataac

SEQ ID NO: 127 - DNA, 59 bp, Synthetic construct
  source 1..59: mol_type = other DNA
aagcttgcatgcctgcaggtcgactctagaggatcccctaagatattcaagaggaaatc

SEQ ID NO: 128 - DNA, 59 bp, Synthetic construct
  source 1..59: mol_type = other DNA
gttgtaaaacgacggccagtgaattcgagctcggtaccggtaccagaatgtgggctttc

SEQ ID NO: 129 - DNA, 38 bp, Synthetic construct
  source 1..38: mol_type = other DNA
tactaacgccgccatccagtcgtgaggcgaggcagatc

SEQ ID NO: 130 - DNA, 38 bp, Synthetic construct
  source 1..38: mol_type = other DNA
aagatctgcctcgcctcacgactggatggcggcgttag

SEQ ID NO: 131 - DNA, 38 bp, Synthetic construct
  source 1..38: mol_type = other DNA
caaagggaccgtggctgcatatcaagcttgcctcgtcc

SEQ ID NO: 132 - DNA, 38 bp, Synthetic construct
  source 1..38: mol_type = other DNA
ggggacgaggcaagcttgatatgcagccacggtccctt

SEQ ID NO: 133 - DNA, 58 bp, Synthetic construct
  source 1..58: mol_type = other DNA
gcttgcatgcctgcaggtcgactctagaggatccggtttacgttgccacactattctg

SEQ ID NO: 134 - DNA, 60 bp, Synthetic construct
  source 1..60: mol_type = other DNA
acgttgtaaaacgacggccagtgaattcgagctcggtacccagtggagcggaaactcttc

SEQ ID NO: 135 - DNA, 60 bp, Synthetic construct
  source 1..60: mol_type = other DNA
actgctgtcgattcgatactaacgccgccatccagttgtggaggtgaagttttaagatag

SEQ ID NO: 136 - DNA, 60 bp, Synthetic construct
  source 1..60: mol_type = other DNA
ctggccgggtgacccggcggggacgaggcaagcttgatttctaatgactgatactcatac

SEQ ID NO: 137 - DNA, 58 bp, Synthetic construct
  source 1..58: mol_type = other DNA
cgccaagcttgcatgcctgcaggtcgactctagaggatccgtggacctgatttgactg

SEQ ID NO: 138 - DNA, 39 bp, Synthetic construct
  source 1..39: mol_type = other DNA
gtgaattcgagctcggtacccctgaatctctcaacgaag

SEQ ID NO: 139 - DNA, 40 bp, Synthetic construct
  source 1..40: mol_type = other DNA
tactaacgccgccatccagttcagaacatggcaaataatg

SEQ ID NO: 140 - DNA, 38 bp, Synthetic construct
  source 1..38: mol_type = other DNA
accacaatcgcttacaccacggatcctctagagtcgac

SEQ ID NO: 141 - DNA, 38 bp, Synthetic construct
  source 1..38: mol_type = other DNA
tcttcgttgagagattcaggggtaccgagctcgaattc

SEQ ID NO: 142 - DNA, 58 bp, Synthetic construct
  source 1..58: mol_type = other DNA
tatactgctgtcgattcgatactaacgccgccatccagttgacggtgtcactttcgag

SEQ ID NO: 143 - DNA, 58 bp, Synthetic construct
  source 1..58: mol_type = other DNA
gtcaagactgtcaaggagggtattctgggcctccatgtcgttgttgatggggaactac

SEQ ID NO: 144 - DNA, 59 bp, Synthetic construct
  source 1..59: mol_type = other DNA
acgttgtaaaacgacggccagtgaattcgagctcggtaccgtcgggttcctgagaaatg

SEQ ID NO: 145 - DNA, 60 bp, Synthetic construct
  source 1..60: mol_type = other DNA
ctatactgctgtcgattcgatactaacgccgccatccagtgggaggaatttgggtggatg

SEQ ID NO: 146 - DNA, 58 bp, Synthetic construct
  source 1..58: mol_type = other DNA
cgctggccgggtgacccggcggggacgaggcaagcttgatacaccgtgaggtgacacc

SEQ ID NO: 147 - DNA, 60 bp, Synthetic construct
  source 1..60: mol_type = other DNA
caagcttgcatgcctgcaggtcgactctagaggatccgcatactttcttcacagttggtg

SEQ ID NO: 148 - DNA, 60 bp, Synthetic construct
  source 1..60: mol_type = other DNA
ctggccgggtgacccggcggggacgaggcaagcttgatgtcatgtaaatattcttagagc

SEQ ID NO: 149 - DNA, 58 bp, Synthetic construct
  source 1..58: mol_type = other DNA
cgccaagcttgcatgcctgcaggtcgactctagaggatccccgtattctagggatgtc

SEQ ID NO: 150 - DNA, 58 bp, Synthetic construct
  source 1..58: mol_type = other DNA
acgttgtaaaacgacggccagtgaattcgagctcggtacccaccgactttttcaaggc

SEQ ID NO: 151 - DNA, 58 bp, Synthetic construct
  source 1..58: mol_type = other DNA
ctatactgctgtcgattcgatactaacgccgccatccagtccaggagtggtaacggtg

SEQ ID NO: 152 - DNA, 59 bp, Synthetic construct
  source 1..59: mol_type = other DNA
gacgttgtaaaacgacggccagtgaattcgagctcggtacccctgtcaagatatttggc

SEQ ID NO: 153 - DNA, 45 bp, Synthetic construct
  source 1..45: mol_type = other DNA
cggcggggacgaggcaagcttgatgttttgctttatattgaactc

SEQ ID NO: 154 - DNA, 45 bp, Synthetic construct
  source 1..45: mol_type = other DNA
gagttcaatataaagcaaaacatcaagcttgcctcgtccccgccg

SEQ ID NO: 155 - DNA, 45 bp, Synthetic construct
  source 1..45: mol_type = other DNA
cacaacgaaagaattgctgtccactggatggcggcgttagtatcg

SEQ ID NO: 156 - DNA, 45 bp, Synthetic construct
  source 1..45: mol_type = other DNA
cgatactaacgccgccatccagtggacagcaattctttcgttgtg

SEQ ID NO: 157 - DNA, 59 bp, Synthetic construct
  source 1..59: mol_type = other DNA
caagcttgcatgcctgcaggtcgactctagaggatccgtttcgatgcctcagggtaatc

SEQ ID NO: 158 - DNA, 51 bp, Synthetic construct
  source 1..51: mol_type = other DNA
cgacggccagtgaattcgagctcggtaccgccacagcgtgacgtgaatttc

SEQ ID NO: 159 - DNA, 51 bp, Synthetic construct
  source 1..51: mol_type = other DNA
gctgtcgattcgatactaacgccgccatccagtgggatgctgggtaggacg

SEQ ID NO: 160 - DNA, 50 bp, Synthetic construct
  source 1..50: mol_type = other DNA
gggtgacccggcggggacgaggcaagcttgatgaggttttgggacgttgg

SEQ ID NO: 161 - DNA, 52 bp, Synthetic construct
  source 1..52: mol_type = other DNA
gcttgcatgcctgcaggtcgactctagaggatcctgtgtgggattagctttg

SEQ ID NO: 162 - DNA, 20 bp, Synthetic construct
  source 1..20: mol_type = other DNA
gcttttacgaccttatatgc

SEQ ID NO: 163 - DNA, 20 bp, Synthetic construct
  source 1..20: mol_type = other DNA
acaaactgtggaattgacgc

SEQ ID NO: 164 - DNA, 44 bp, Synthetic construct
  source 1..44: mol_type = other DNA
gttacccatagcattaatgtggttttagagctagaaatagcaag

SEQ ID NO: 165 - DNA, 46 bp, Synthetic construct
  source 1..46: mol_type = other DNA
cacattaatgctatgggtaacaaattaaaaatagtttacgcaagtc

SEQ ID NO: 166 - DNA, 20 bp, Synthetic construct
  source 1..20: mol_type = other DNA
gactgtcaaggagggtattc

SEQ ID NO: 167 - DNA, 20 bp, Synthetic construct
  source 1..20: mol_type = other DNA
ccgcaagtgattagacttag

SEQ ID NO: 168 - DNA, 20 bp, Synthetic construct
  source 1..20: mol_type = other DNA
gaataccacttgtttaccgg

SEQ ID NO: 169 - DNA, 20 bp, Synthetic construct
  source 1..20: mol_type = other DNA
ggtggcggcaaaactaattc

SEQ ID NO: 170 - DNA, 20 bp, Synthetic construct
  source 1..20: mol_type = other DNA
cgctatatccttcttggtcg

SEQ ID NO: 171 - DNA, 20 bp, Synthetic construct
  source 1..20: mol_type = other DNA
gtcccaaaaccttctcaagc

SEQ ID NO: 172 - DNA, 59 bp, Synthetic construct
  source 1..59: mol_type = other DNA
catcagaattgttcaaacaactaccaattgttaatatcagggaaacagctatgaccatg

SEQ ID NO: 173 - DNA, 58 bp, Synthetic construct
  source 1..58: mol_type = other DNA
gaactagatcaagccaaaaaggtgatcataacaatatagtcagtaaaacgacggccag

SEQ ID NO: 174 - DNA, 46 bp, Synthetic construct
  source 1..46: mol_type = other DNA
cctggtggtgcagttacagtcaaattaaaaatagtttacgcaagtc

SEQ ID NO: 175 - DNA, 44 bp, Synthetic construct
  source 1..44: mol_type = other DNA
gactgtaactgcaccaccagggttttagagctagaaatagcaag

SEQ ID NO: 176 - DNA, 58 bp, Synthetic construct
  source 1..58: mol_type = other DNA
acgttgtaaaacgacggccagtgaattcgagctcggtacccagataactcaccacgag

SEQ ID NO: 177 - DNA, 40 bp, Synthetic construct
  source 1..40: mol_type = other DNA
ggggacgaggcaagcttgatgaaagatgatgggaaacaag

SEQ ID NO: 178 - DNA, 38 bp, Synthetic construct
  source 1..38: mol_type = other DNA
cttgtttcccatcatctttcatcaagcttgcctcgtcc

SEQ ID NO: 179 - DNA, 46 bp, Synthetic construct
  source 1..46: mol_type = other DNA
gcaagcgaagcaagtttcatttttgttagtttttgaggtgatacag

SEQ ID NO: 180 - DNA, 39 bp, Synthetic construct
  source 1..39: mol_type = other DNA
cacctcaaaaactaacaaaaatgaaacttgcttcgcttg

SEQ ID NO: 181 - DNA, 59 bp, Synthetic construct
  source 1..59: mol_type = other DNA
cgccaagcttgcatgcctgcaggtcgactctagaggatccgtctacagtggttgagaac

SEQ ID NO: 182 - DNA, 40 bp, Synthetic construct
  source 1..40: mol_type = other DNA
ccggtcttcttttgctctaagtagcagcatgattatgaac

SEQ ID NO: 183 - DNA, 40 bp, Synthetic construct
  source 1..40: mol_type = other DNA
agaagaagactgtatttcattgtggaggtgaagttttaag

SEQ ID NO: 184 - DNA, 42 bp, Synthetic construct
  source 1..42: mol_type = other DNA
cttaaaacttcacctccacaatgaaatacagtcttcttcttc

SEQ ID NO: 185 - DNA, 38 bp, Synthetic construct
  source 1..38: mol_type = other DNA
gttcataatcatgctgctacttagagcaaaagaagacc

SEQ ID NO: 186 - DNA, 60 bp, Synthetic construct
  source 1..60: mol_type = other DNA
ggccgggtgacccggcggggacgaggcaagcttgattgtggaggtgaagttttaagatag

SEQ ID NO: 187 - DNA, 59 bp, Synthetic construct
  source 1..59: mol_type = other DNA
ctatactgctgtcgattcgatactaacgccgccatccagtcacatgggctccgtttctg

SEQ ID NO: 188 - DNA, 52 bp, Synthetic construct
  source 1..52: mol_type = other DNA
agaagtaaagagaatctcatttttgttagtttttgaggtgatacagaaaaag

SEQ ID NO: 189 - DNA, 43 bp, Synthetic construct
  source 1..43: mol_type = other DNA
cacctcaaaaactaacaaaaatgagattctctttacttcttgc

SEQ ID NO: 190 - DNA, 58 bp, Synthetic construct
  source 1..58: mol_type = other DNA
cgccaagcttgcatgcctgcaggtcgactctagaggatccgcaagacgaacctccaac

SEQ ID NO: 191 - DNA, 58 bp, Synthetic construct
  source 1..58: mol_type = other DNA
tcgttaactgtgtattacctgttcataatcatgctgctacttaaagcaacaaggcagc

SEQ ID NO: 192 - DNA, 23 bp, Synthetic construct
  source 1..23: mol_type = other DNA
gtagcagcatgattatgaacagg

SEQ ID NO: 193 - DNA, 60 bp, Synthetic construct
  source 1..60: mol_type = other DNA
tgctgtcgattcgatactaacgccgccatccagtttctaatgactgatactcatactttc

SEQ ID NO: 194 - DNA, 18 bp, Synthetic construct
  source 1..18: mol_type = other DNA
ttaaagcaacaaggcagc

SEQ ID NO: 195 - DNA, 50 bp, Synthetic construct
  source 1..50: mol_type = other DNA
tacacctgcaagaagtaaagagaatctcattgtggaggtgaagttttaag

SEQ ID NO: 196 - DNA, 49 bp, Synthetic construct
  source 1..49: mol_type = other DNA
ctttgcttgctgctgccttgttgctttaagtagcagcatgattatgaac

SEQ ID NO: 197 - DNA, 42 bp, Synthetic construct; identical to SEQ ID NO: 36
  source 1..42: mol_type = other DNA

SEQ ID NO: 198 - DNA, 44 bp, Synthetic construct; identical to SEQ ID NO: 37
  source 1..44: mol_type = other DNA

SEQ ID NO: 199 - DNA, 21 bp, Synthetic construct
  source 1..21: mol_type = other DNA
cagttaatagtttgcgcaacg

SEQ ID NO: 200 - DNA, 19 bp, Synthetic construct
  source 1..19: mol_type = other DNA
ctgtagcaatggcaacaac

SEQ ID NO: 201 - DNA, 57 bp, Synthetic construct
  source 1..57: mol_type = other DNA
ccggcgttgtgtgctgtctcccacatgctttggaggttcgtcttgctttggagccag

SEQ ID NO: 202 - DNA, 59 bp, Synthetic construct
  source 1..59: mol_type = other DNA
gaacctccaaagcatgtgggagacagcacacaacgccggctggtttgtgcttgtgtctc

SEQ ID NO: 203 - DNA, 60 bp, Synthetic construct
  source 1..60: mol_type = other DNA
aacggatgctcgaggctgctcacgctcacgctccagccggcgttgtgtgctgtctcccac

SEQ ID NO: 204 - DNA, 60 bp, Synthetic construct
  source 1..60: mol_type = other DNA
ccggctggagcgtgagcgtgagcagcctcgagcatccgttccgacctcactctcactctg

SEQ ID NO: 205 - DNA, 48 bp, Synthetic construct
  source 1..48: mol_type = other DNA
gccatcatgctgcttccaacgatggccccgagtttatcgatgcgggag

SEQ ID NO: 206 - DNA, 48 bp, Synthetic construct
  source 1..48: mol_type = other DNA
ggggccatcgttggaagcagcatgatggccacctttcacccagtagtg